(19) United States
(12) Patent Application Publication
(10) Pub. No.: US 2019/0346442 A1
(43) Pub. Date: Nov. 14, 2019

(54) IMPROVED HLA EPITOPE PREDICTION

(71) Applicants: The Broad Institute, Inc., Cambridge, MA (US); Massachusetts Institute of Technology, Cambridge, MA (US); President and Fellows Harvard College, Cambridge, MA (US); Dana Farber Cancer Institute, Inc., Boston, MA (US); The General Hospital Corporation, Boston, MA (US)

(72) Inventors: Steven A. Carr, Boxford, MA (US); Nir Hacohen, Brookline, MA (US); Catherine J. Wu, Brookline, MA (US); Jennifer G. Abelin, Somerville, MA (US); Siranush Sarkizova, Cambridge, MA (US); Derin B. Keskin, Newton, MA (US); Karl R. Clauser, Boston, MA (US); Michael S. Rooney, Cambridge, MA (US)

(21) Appl. No.: 16/094,786

(22) PCT Filed: Apr. 18, 2017
(86) PCT No.: PCT/US2017/028122

§ 371 Date: Oct. 18, 2018

Related U.S. Application Data
(60) Provisional application No. 62/324,228, filed on Apr. 18, 2016.
(60) Provisional application No. 62/345,556, filed on Jun. 3, 2016.
(60) Provisional application No. 62/458,954, filed on Feb. 14, 2017.

(51) Int. Cl. G01N 33/569; C12Q 1/6881; G16B 40/10; G16B 30/00; G16B 25/10; C12Q 1/6886; A61K 39/00

(52) U.S. Cl. CPC G01N 33/56977; C12Q 1/6881; G16B 40/10; G16B 30/00; G01N 2560/00; C12Q 1/6886; A61K 39/0011; A61K 2039/5158; G16B 25/10

(57) ABSTRACT

Adaptive immune responses rely on the ability of cytotoxic T cells to identify and eliminate cells displaying disease-specific antigens on human leukocyte antigen (HLA) class I molecules. Investigations into antigen processing and display have immense implications in human health, disease and therapy. To extend understanding of the rules governing antigen processing and presentation, immunopurified peptides from B cells, each expressing a single HLA class I allele, were profiled using accurate mass, high-resolution liquid chromatography-mass spectrometry (LC-MS/MS). A resource dataset containing thousands of peptides bound to 28 distinct class I HLA-A, -B, and -C alleles was generated by implementing a novel allele-specific database search strategy. Applicants discovered new binding motifs, established the role of gene expression in peptide presentation and improved prediction of HLA-peptide binding by using these data to train machine-learning models. These streamlined experimental and analytic workflows enable direct identification and analysis of endogenously processed and presented antigens.

### RELATED APPLICATIONS AND INCORPORATION BY REFERENCE

This application is a national stage filing under 35 U.S.C. § 371 of PCT International Application No. PCT/US2017/028122, filed Apr. 18, 2017, which claims priority and benefit of U.S. Provisional application Ser. No. 62/324,228 filed Apr. 18, 2016, 62/345,556 filed Jun. 3, 2016, and 62/458,954 filed Feb. 14, 2017, the contents of both of which are incorporated herein by reference in their entirety.

The foregoing applications, and all documents cited therein or during their prosecution (“appin cited documents”) and all documents cited or referenced in the appin cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under grant numbers CA155010, CA160034 and HG002295 awarded by the National Institutes of Health. The government has certain rights in the invention.

### FIELD OF THE INVENTION

The present application relates to methods for improved prediction of HLA-peptide binding, datasets for predicting HLA-peptide binding and selection of HLA-binding peptides and compositions comprising HLA-binding peptides obtained by these methods.

### BACKGROUND OF THE INVENTION

The HLA Class I proteins (HLA-A, B and C) are expressed on the surface of almost all nucleated cells in the human body and are required for presentation of short peptides for detection by T cell receptors. The HLA-bound peptides arise from endogenous or foreign proteins cleaved by the proteasome and ER peptidases and loaded on HLA Class I proteins. The HLA genes are the most polymorphic genes across the human population, with more than 10,000 HLA class I allele variants identified to date (6; IPD-IMGT/HLA database Release 3.24.0.1). Each HLA allele is estimated to bind and present ˜1,000-10,000 unique peptides to T cells (1-5) (≤0.1% of ˜10 million potential 9mer peptides from human protein-coding genes). The peptide-binding rules are only known for a relatively limited set of common alleles (5), and have been encoded in algorithms that predict the binding of an arbitrary peptide to specific HLA alleles, and thus accelerate the discovery of epitopes.

Personalized immunotherapy using tumor-specific peptides has been described (Ott et al., Hematol. Oncol. Clin. N. Am. 28 (2014) 559-569). Efficiently choosing which particular peptides to utilize as an immunogen requires the ability to predict which tumor-specific peptides would efficiently bind to the HLA alleles present in a patient. Neural network based learning approaches with validated binding and non-binding peptides have advanced the accuracy of prediction algorithms for the major HLA-A and -B alleles (Zhang et al, Machine learning competition in immunology—Prediction of HLA class I binding peptides, J Immunol Methods 374:1 (2011); Lundegaard et al., Prediction of epitopes using neural network based methods, J Immunol Methods 374:26 (2011)).

Even using advanced neural network-based algorithms to encode HLA-peptide binding rules (7, 8), several factors limit the power to predict peptides presented on HLA alleles. First, the provenance of peptide data upon which these algorithms are trained is diverse, ranging from peptide library screens to Edman degradation and only sometimes endogenous peptides (3-5, 9). In fact, the algorithms most commonly used today are trained almost exclusively on measurements of biochemical affinity of synthetic peptides (Trolle et al., Automated benchmarking of peptide-MHC class I binding predictions, Bioinformatics, 2015 Jul. 1, 31(13):2174-2181). Second, many existing prediction algorithms have focused on predicting binding but may not fully take into account endogenous processes that generate and transport peptides prior to binding (10). Third, the number of binding peptides for many HLA alleles is too small to develop a reliable predictor. Until now, however, the generation of high-quality resource datasets has been hampered by inefficient protocols that necessitate prohibitively large amounts of input cellular material, and a lack of database search tools for HLA-peptide sequencing (5, 7, 8, 11).

Thus, there is a need for improved tools and methods for prediction of antigen presentation.

Citation or identification of any document in this application is not an admission that such document is available as prior art to the present invention.

### SUMMARY OF THE INVENTION

One objective of the present is to provide an improved tool for predicting peptides that are presented by HLA proteins. Another objective of the present invention is to provide peptides capable of inducing an immune response upon administration to a subject.

In one aspect, the invention provides methods of generating an HLA-allele specific binding peptide sequence database comprising:
(a) providing a population of cells expressing a single HLA allele;
(b) isolating HLA-peptide complexes from said cells;
(c) isolating peptides from said HLA-peptide complexes; and
(d) sequencing said peptides.

In particular embodiments, the methods are methods of generating an HLA class I-allele specific binding peptide sequence database comprising:
(a) providing a population of cells expressing a single HLA class I allele;
(b) isolating class I HLA-peptide complexes from said cells;
(c) isolating peptides from said HLA-peptide complexes; and
(d) sequencing said peptides.

In particular embodiments the methods are methods of generating an HLA class II-allele specific binding peptide sequence database comprising:
(a) providing a population of cells expressing a pair of HLA Class II genes, consisting of one α and one β subunit;
(b) isolating class II HLA-peptide complexes from said cells;
(c) isolating peptides from said HLA-peptide complexes; and
(d) sequencing said peptides.

In particular embodiments, said sequencing is ensured by LC-MS/MS.

In particular embodiments, the population of cells comprises at least 10⁷ cells.

In particular embodiments, the cells are dendritic cells, macrophages or B-cells.

In particular embodiments, the cells are tumor cells.

In particular embodiments, the cells are contacted with an agent or condition prior to isolating said HLA-peptide complexes from said cells. In particular embodiments, said agent or condition is an inflammatory cytokines, a chemical agent, a therapeutic agent or radiation.

In particular embodiments, the HLA allele is a mutated HLA allele. In particular embodiments, the HLA allele is selected from A*01:01, A*02:01, A*02:03, A*02:04, A*02:07, A*03:01, A*24:02, A*29:02, A*31:01, A*68:02, B*35:01, B*44:02, B*44:03, B*51:01, B*54:01, B57:01, C*03:02, C*03:04, C*04:01, C*05:01, C*06:02, C*08:01, C*08:02, C*12:02, C*14:02, C*14:03, C*15:02, and C*16:01.

In particular embodiments, step (b) comprises lysing the cells and isolating the HLA-peptide complexes by immunoprecipitation.

In particular embodiments, the methods involve carrying out steps (a) to (d) subsequently for different HLA alleles.

In a further aspect the application provides HLA-allele specific binding peptide sequence databases obtained by carrying out the methods as described herein. Further, the application provides combinations of two or more HLA-allele specific binding peptide sequence databases obtained by carrying out the methods as described herein, each time using a different HLA-allele.

In a further aspect, the application provides methods for generating a prediction algorithm for identifying HLA-allele specific binding peptides, which methods comprise training a machine with the peptide sequence database or the combinations of peptide sequence databases described herein. In particular embodiments of the methods provided herein, the machine combines one or more linear models, support vector machines, decision trees and neural networks. In particular embodiments, the variables used to train the machine comprise one or more variables selected from the group consisting of peptide sequence, peptide upstream and downstream sequence, amino acid physical properties, amino acid similarity, peptide physical properties, expression level of the source protein of a peptide within a cell, various properties of peptide source, e.g., protein/transcript length, cell localization, GC content, number of exons, disorder quantification, ubiquination sites, etc., and peptide cleavability. The application further provides a prediction algorithm for identifying HLA-allele specific binding peptides generated by the methods described herein.

The application further provides methods for identifying HLA-allele specific binding peptides, which method comprises analyzing the sequence of a peptide with a machine which has been trained with a peptide sequence database obtained by carrying out the methods for predicting the binding of peptides to said HLA-protein described herein. In particular embodiments, the methods comprise: determining the expression level of the source protein of the peptide within a cell; wherein the source protein expression is one of the predictive variables used by the machine. In particular embodiments, the expression level is determined by measuring the amount of source protein or the amount of RNA encoding said source protein.

In a further aspect, the application provides methods of identifying from a given set of neo-antigen comprising peptides the most suitable peptides for preparing an immunogenic composition for a subject, said method comprising selecting from a given set of peptides the plurality of peptides capable of binding an HLA protein of the subject, wherein said ability to bind an HLA protein is determined by analyzing the sequence of peptides with a machine which has been trained with peptide sequence databases corresponding to the specific HLA-binding peptides for each of the HLA-alleles of said subject.

The application further provides methods of identifying from a given set of neo-antigen comprising peptides the most suitable peptides for preparing an immunogenic composition for a subject, said method comprising selecting from set given set of peptides the plurality of peptides determined as capable of binding an HLA protein of the subject, ability to bind an HLA protein is determined by analyzing the sequence of peptides with a machine which has been trained with a peptide sequence database obtained by carrying out the methods for identifying HLA-allele specific binding peptides as described herein.

The application further provides methods of identifying a plurality of subject-specific peptides for preparing a subject-specific immunogenic composition, wherein the subject has a tumor and the subject-specific peptides are specific to the subject and the subject's tumor, said method comprising: (a) whole genome or whole exome nucleic acid sequencing of a sample of the subject's tumor and a non-tumor sample of the subject; (b) determining based on the whole genome or whole exome nucleic acid sequencing: (i) non-silent mutations present in the genome of cancer cells of the subject but not in normal tissue from the subject, and (ii) the HLA genotype of the subject; wherein the non-silent mutations comprise a point, splice-site, frameshift, read-through, new open reading frame (neoOFR), or gene-fusion mutation; said method further comprising step (c) selecting from the identified non-silent mutations the plurality of subject-specific peptides, each having a different tumor neo-epitope that is an epitope specific to the tumor of the subject and each having a predictive score indicative of processing and binding an HLA protein of the subject, wherein said predictive score is determined by analyzing peptides (e.g., analyzing the sequence, context and properties of peptides) derived from the non-silent mutations by carrying out the methods for identifying HLA-allele specific binding peptides described herein.

The application further provides methods of identifying a plurality of subject-specific peptides for preparing a subject-specific immunogenic composition, said method comprising selecting a plurality of subject-specific peptides, each having a different tumor neo-epitope that is an epitope specific to the tumor of the subject and each having a predictive score indicative of binding an HLA protein of the subject, wherein said predictive score is determined by analyzing the peptides (e.g., analyzing the sequence, context and properties of peptides) derived from the non-silent mutations by carrying out the methods for identifying HLA-allele specific binding peptides described herein.

In a further aspect, the invention provides, immunogenic compositions for use in a method of inducing a tumor specific immune response, said immunogenic composition comprising two or more peptides identified with the method according to the methods provided herein and a pharmaceutically acceptable carrier. In particular embodiments, the application provides immunogenic composition for use in a method of inducing a tumor specific immune response, comprising autologous dendritic cells or antigen presenting cells that have been pulsed with the two or more peptides identified with the method according to the methods provided herein. The application further provides immunogenic compositions for use in a method of inducing a tumor specific immune response, comprising at least one vector capable of expressing the two or more peptides identified with the methods for identifying subject-specific peptides for preparing a subject-specific immunogenic compositions described herein. In particular embodiments, the vector is a viral vector. The present invention also encompasses immunogenic compositions comprising one or more peptides, or one or more vectors expressing the one or more peptides, of Tables 1A, 1B and/or 1C as well as a library comprising the same.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1D illustrates an efficient sample processing and analysis pipeline for HLA-peptide sequencing. A. Overview of the experimental workflow. 721.221 B cells were transfected with single HLA alleles and 30-90 million cells were used for HLA-peptide immunopurifications. Eluted peptides were analyzed with high resolution LC-MS/MS. HLA-associated peptides were sequenced and identified using an HLA allele-specific database search. B. Schema of the HLA-specific database search strategy. The number of peptide spectrum matches (PSMs) identified through this strategy per HLA allele are shown in FIG. 6A, with all peptide identifications provided in Table 2. C. Peptide length distributions from all HLA-A and HLA-B alleles. D. HLA class I-associated peptide identifications from 16 characterized HLA alleles. Total numbers of unmodified (left segment), modified (middle segment), and negative control peptides (right segment) identified per allele are shown (see FIG. 6C for distribution of peptide modifications). Negative control peptides are listed in Table 1E. Allele frequencies among Caucasian, Asian, and Black populations are shown. “*” denotes alleles for which LC-MS/MS experiments have generated a greater number of peptides than reported in IEDB (see FIG. 6D).

FIG. 2A-2F illustrates novel HLA peptide-binding motifs enriched in LC-MS/MS data relative to IEDB A. Average distance comparisons between pairs of 9mer peptides (left bars-LC-MS/MS data; middle bars-IEDB data) presented by a particular allele. The average distance between IEDB and LC-MS/MS peptides, right bars, (see FIG. 7A and B for individual HLA alleles). B. Summary plot of entropy per position across all HLA alleles in LC-MS/MS (bottom) and IEDB (top) datasets. C. Sequence logos comparing the HLA-binding motifs for HLA-A*02:01 and -A*29:02-associated 9mers sequenced by LC-MS/MS (left) and reported by IEDB (right). D. Systematic evaluation of the frequencies of each amino acid (positions 1-9) within 9mers sequenced by LC-MS/MS for the 13 of 16 HLA alleles for which IEDB data has reported. Orange/light—Amino acids overrepresented in LC-MS/MS data (scaled by p-value); blue/dark—amino acids underrepresented in LC-MS/MS data (scaled by p-value). E,F. Non-metric multidimensional scaling (NMDS) was used to visualize peptide distances in two dimensions for each analyzed HLA allele (FIG. 8), with examples provided for HLA-A*02:01 (E, top) and -A*29:02-associated peptides (F, top). Each circle represents a unique 9mer peptide from either the LC-MS/MS (orange/light) or IEDB (blue/dark) datasets, with the size of each circle proportional to a peptide's NetMHCpan-2.8 predicted binding affinity. Sequence logos representing these LC-LC-MS/MS and IEDB data are also shown for the highlighted peptide clusters presented by HLA-A*02:01(E, bottom) and HLA-A*29:02 (F, bottom).

FIG. 3A-3D illustrates analysis of peptide cleavage signatures and MHC-binding registers. A. The cleavage specificity of the proteasome represented by the percent change from background in amino acid frequencies upstream (U1-U6) and downstream (D1-D6) of the N- and C-termini of peptides (average over 16 HLA alleles). Amino acid positions are colored according to the directionality and significance of the enrichment. B. Cleavability scores based on amino acid enrichments and depletions upstream (‘N-terminal scoring’-green) and downstream (‘C-terminal scoring’-black) of HLA-presented peptides (“hits”) and a set of 1×10⁶ random genomic 9mers (“decoys”). The low average ratio of hit:decoy cleavability scores at internal peptide positions illustrate below-average cleavability, while high ratios at the N- and C-termini illustrate high cleavability. C. Peptides sequenced by LC-MS/MS (“hits”-red/right) appeared significantly more cleavable than decoys (purple/left) when scored by a novel peptide cleavability model based on observations in 3A and 3B (see Methods). An analogous analysis was performed using the tool NetChop(FIG. 9A). D. The observed number of peptides at each position (relative distance from protein N-terminus) compared to the expected number, assuming each MS-observed peptide was equally likely to have arisen from any position in its source protein (black solid line). Red dashed line—the expected result if a large proportion of HLA-presented peptides arose from aborted translation products.

FIG. 4A-4G illustrates evaluation of HLA-peptide characteristics that impact HLA-binding predictions. A. Distributions of NetMHCpan2.8-predicted HLA-binding affinities of peptides identified by LC-MS/MS (“hits”; left peak) compared to 1×10⁶ random 9mer peptides from protein-coding genes (“decoys”; right peak). B. Distributions of source RNA transcript expression (summed transcripts for each gene) of hits vs. decoys peptides. C. Hits and decoys binned according to expression (y-axis) and predicted affinity (x-axis) for each allele and summed. Hit (top) and decoy (bottom) counts are reported for each bin, which is colored according to the hit:decoy ratio (red/upper left=hits>decoys; blue/lower right hits<decoys). Bins with the same expression:affinity ratio that demonstrate roughly equivalent hit:decoy ratio are highlighted (orange-Group A peptides with high expression ; white-Group C peptides with low expression). D. Cellular localization of HLA-associated peptide source proteins are reported as a frequency relative to expression-matched decoy peptides. The same analysis without expression-matching is shown in FIG. 9B. E. NetMHCStab predicted peptide-binding stability of peptides sequenced by LC-MS/MS and affinity- and expression-matched decoys (p-values by t-test; all alleles FIG. 9C). F. Approximately 200 protein-protein interaction experiments (Behrends et al., 2010, Nature 466, 68-76; Christianson et al., 2012, Nat. Cell Biol. 14, 93-105; Sowa et al., 2009, Cell 138, 389-403), each yielding set of 50-100 high confidence interacting proteins for a given bait (usually a known protein turnover pathway gene) were scored according to their enrichment for LC-MS/MS-observed peptides, here depicted as a histogram. Each block corresponds to one experiment and is colored according to the directionality and significance (chi-square test) of the enrichment (see key). The bait protein used in outlier experiments (SQSTM1, PIK3C3, and OTUD4) is marked along with corresponding p-value. G. Percent change in amino acid frequency of top-scoring peptides (top 25%) compared to bottom-scoring peptides (bottom 25%) amongst 1 million random proteome 9mers evaluated by NetChop (Saxová et al., 2003, Int. Immunol. 15, 781-787). Color coding indicates directionality and magnitude of percent change (see key).

FIG. 5A-5H illustrates evaluation of novel MS-based HLA-peptide binding predictors. A. MS 9mer peptides (orange/light) compared to IEDB 9mer peptides (blue/dark). Non-metric multidimensional scaling (NMDS) was used to visualize pairwise peptide distances in two dimensions for each analyzed HLA allele. Peptide distance was defined based on sequence similarity (Kim et al., Derivation of an amino acid similarity matrix for peptide: MHC binding and its application as a Bayesian prior, BMC Bioinformatics, 10, 1-11, 2009). The size of each circle corresponds to the NetMHCpan-predicted affinity score of the corresponding peptide. B. Experimental validation of MS-based models. Per-allele generalized linear models (trained on LC-MS/MS sequenced peptides and random 9mer peptides from protein-coding genes), NetMHCpan-2.8, and NetMHC-4.0 were used to predict the LC-MS/MS data. Peptides scoring in the top 10% by the MS predictor but the bottom 10% by NetMHC-2.8 were selected for experimental validation. All successfully synthesized peptides for 4/5 alleles are visualized on NMDS plots (A) and numbered according to the corresponding line in the table of measured and predicted binding probability (MS) or affinities (NetMHC) (for each cell line, the data shown across the four bars is, from left to right, NetMHC-4.0, NetMHCpan-2.8, MS Intrinsic, and MS IntrinsicEC.) (B) (see FIG. 10A for HLA-B*35:01). The peptide which failed experimental validation is: YIIEREPLI. C. Saturation analysis. For each allele, neural network models with peptide-intrinsic features and dummy sequence encoding only were built with increasing number of positive training examples, from 15 to the total number peptides identified by LC-MS/MS per allele. The PPV for each model was evaluated and plotted as a function of the number of binders in the training set. Allele complexity scores, defined as a weighted average of the entropy at each peptide position, are shown in the figure legend (Methods, FIG. 7A-B). D. Internal evaluation. Average PPV (top) and AUC (bottom) achieved by NetMHC-2.8, NetMHC-4.0, and the two MS-based ensembles on LC-MS/MS dataset. E. Positive predictive value of linear models used to discern 9mer MS peptides amongst a 999-fold excess of 9mer decoys (averaging across 16 alleles). Models included one or more predictor variables (A=affinity, S=stability, R=RNA-Seq expression, P=protein expression (iBAQ), C=cleavability score, L=source protein localization). F. Explanatory contributions of predictor variables derived by monitoring the cumulative improvement in predictive value as predictors are added. G. Cartoon representation of the neural network model architecture. The 215 MSIntrinsic inputs included an amino acid encoding (180 nodes), amino acids properties (27 nodes), and peptide properties (8 nodes). The 182 MSIntrinsicEC inputs included the amino acid encoding, expression (1 node), and cleavability (1 node). H. External evaluation. In addition to a standardized competition dataset and a HIV epitope dataset (see Table 4) MS-binding data from an independent high-throughput published dataset consisting of 6 multi-allele cell lines (1) was used to compare the performance of MSIntrinsic and MSIntrinsicEC, and neural networks against NetMHC-2.8 and NetMHC-4.0 (FIG. 10D). Evaluations were performed for all alleles that overlap with our data. For each cell line and overlapping allele combination, binders to other alleles in the cell line were removed from the evaluation set if they had NetMHCpan-2.8 predicted binding affinity <500 nM for another allele and >1000 nM for the allele being evaluated. Peptides which did not have a match in the transcriptome of the sequencing data were also excluded to allow for a direct comparison between MSIntrinsic and MSIntrinsicEC. PPV was calculated after combining the remaining hits with 999 n random decoys. (First bars correspond to NetMHC-4.0 data; Second bars correspond to NetHMCpan-2.8 data; third bars correspond to MS Intrinsic data; and fourth bars correspond to MS Intrinsic EC data).

FIG. 6A-6E: A. The number of peptide spectrum matches (PSMs) identified from both the no enzyme and HLA-specific rounds of database searches are shown for each HLA allele dataset. These PSMs represent the unique peptide identifications reported in Table 2. B. The overlap of unique peptides identified from biological replicates of our LC-MS/MS data (orange) and published data (purple) (3) generated from immunopurifications of HLA-A*02:01 expressing cells. Unique peptide overlap between our HLA-A*02:01 dataset and this published dataset is also shown. C. The distribution of peptide modifications represented by the “Modified peptides” category in FIG. 1 is shown as a pie chart. Peptide modifications included oxidized Met (m), deamidation (n), N-term Pyroglutamate (q), phosphorylation (sty), and cysteinylation (c). D. A bar plot comparing the total number of unique peptide sequences reported in IEDB to the number of unique peptides identified using the LC-MS/MS-based workflow. (Total (control removed)—top bars; IEDB peptides—bottom bars). E. The average amino acid frequencies observed across both IEDB and LC-MS/MS datasets compared to the natural amino acid frequencies calculated from the UCSC protein database used for proteomic database searches. The average amino acid frequencies across all 9mers within IEDB and the MS datasets were calculated after removing both position 2 and the last position anchors.

FIG. 7A-7B: A. Sequence logos generated using 9mer data for the 28 HLA alleles characterized by LC-MS/MS. B. Individual allele entropy calculations for each amino acid positions within 9mer peptides sequenced by LC-MS/MS (entropy is normalized by log(20) and shown on to [0,1] scale).

FIG. 8: NMDS plots showing HLA-associated 9mer peptide clustering for individual HLA alleles.

FIG. 9A-9J: A. NetChop cleavability scores of LC-MS/MS identified peptides compared to random decoys.B. Cellular localization of HLA-associated peptide source proteins not corrected for expression. C. NetMHCStab predictions, available at the time of submission, for the alleles HLA-A*01:01, A*02:01, A*03:01. A*24:02, B*35:01. D. Distribution of predicted affinities for the short isoforms (leftmost tall peak) and long isoforms (wide shallow double peak) of nested sets as well as for simulated long isoforms (where random amino acids were added at the beginning or end of the short isoforms, shown in the dark rightmost tall peak). E. MS peptides with high (red) and low (blue) MS1 ion intensities (top and bottom 10%, respectively), plotted by their NetMHCpan-predicted affinity and source transcript expression. F. Each LC-MS/MS identified peptide was matched to ten random proteome 9mer decoys with approximately equal expression but different source genes. The observed count of MS peptides divided by the expected count (based on decoy frequencies) is shown as a function of the number of upstream ATGs. P-values were calculated by t-test. G. Observed vs. expected HLA-peptide counts (using expression-matched decoys) as a function of source protein instability index (Guruprasad et al., 1990, Protein Eng. 4, 155-161). P-values calculated by t-test. H. Similar analysis to (F) showing enrichments as a function of the amount of intrinsically disordered sequence within each peptide's source protein. I. Enrichments according to the count of ubiquitination sites, as previously observed (Kronke et al., 2015, Nature (2015) 523(7559); Kronke et al., 2014, Science (2014) 343(6168); Udeshi et al., 2012, Molecular & Cellular Proteomics (2012) 11: 148-59), within the source protein. J. The observed count of LC-MS/MS identified HLA-peptides mapping to each localization (Uniprot) relative to the expected count relative to random 9mer decoys (left) or expression-matched decoys (right).

FIG. 10A-10D: Machine Learning model performance for individual HLA alleles. A. Experimental validation as in FIG. 5A,B for B*35:01. B. Sequence logos generated for decoys ranked within the top n positions based on ‘MSIntrinsic’ and NetMHC-4.0 evaluations of hits merged with 999 n decoys, where n is the number of binders for the allele in the LC-MS/MS data. C. NMDS visualization of the 10% lowest ranked hits which were not in the top n (false negatives) based on the same evaluation as in B. D. Standard AUC plots are shown per allele for the same evaluation as in B. (left) and AUC zooming into the [0,0.1]% false positive rate (right, where the top two curves are MS intrinsic EC and MS intrinsic, respectively, and bottom two lines are netMHC-4.0 and netMHCpan-2.8).

FIG. 11A-11G: A. HLA cell surface presentation of single-HLA cell lines were compared to primary lymphocytes using FACS analysis. Cell lines that resulted in high (top; HLAA*02:01,-A*02:07) and low (bottom; HLA-A*31:01,-B*35:01) numbers of HLA-associated peptide identifications by LC-MS/MS are shown. The number of total LC-MS/MS peptide identifications correlates with total cell surface HLA presentation. B-G. Heatmaps of amino acid frequencies calculated from external class HLA I datasets, including the class II data from MUTZ3 (Mommen et al., 2016, Mol. Cell. Proteomics MCP 15, 1412-1423) (B), the breast cancer cell line HCC1937 (C), colorectal cell line HCT116 (D), fibroblasts (E), HeLa cells (Bassani-Sternberg et al., 2015, Mol. Cell. Proteomics 14, 658-673) (F), and peripheral blood mononuclear cells (Caron et al., 2015, Mol. Cell. Proteomics, 14(12):3105-17) (G).

FIG. 12A-12F: A. To evaluate LC-MS/MS bias, the “MS Observability Index”, as measured by the ESP algorithm (Fusaro et al., 2009, Nature Biotechnology 27, 190-198), was calculated for IEDB (left most) and MS (right most) peptide datasets. Distributions of the MS observability are displayed. B. Amino acid frequencies within peptides reported in our single-allele dataset are compared to amino acid frequencies in peptides reported in IEDB. C. Amino acid frequency ratios for cleavage-influencing amino acids upstream of, downstream of, and within peptides derived from LC-MS/MS identified peptides compared to random proteome 9mers. D. Enrichment/depletion of protein sequence features among LC-MS/MS peptides. Each MS peptide was matched to 10 random decoy 9mers from the same source transcript. The relative rates at which hits and decoys mapped to Uniprot-defined sequence features (alpha helices, beta strands, signal peptides, and so on) were calculated as ratios and assessed by chi-square test. E. Expression of proteo-some genes in B721.221 cells and in high-purity (>95%) samples from TCGA. Purity was determined ac-cording to the “percent tumor cell” field in the clinical slide review; if more than five samples were of suffi-cient purity for a given tumor type, only the top five were used. The listing in the figure key corresponds, from top to bottom, to the data from left to right in the table bars. For example, PSMB1 is the left most section of each of the bars on the graph. F. Comparison of amino acid frequency between IEDB peptides and Trolle or Mann peptides (Bassani-Sternberg et al., 2015, Mol. Cell. Proteomics 14, 658-673; Trolle et al., 2016, J. Immunol., 196(4):1480-7), respectively. To avoid biases due to anchor residues, for each comparison, 300 peptides per allele were selected at random for the alleles in the corresponding data set (Trolle: A*01:01, A*02:01, A*24:02, B*51:01; Mann: A*01:01, A*02:01, A*03:01, A*24:02, A*3101, B*51:01) and pooled together before amino acid frequency was calculated.

FIG. 13: NMDS plots showing HLA-associated 9mer peptide clustering for a subset of peptides from MS or IEDB with physicochemical properties favorable for MS detection.

### DETAILED DESCRIPTION OF THE INVENTION

Before the present methods of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”. It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention. It may be advantageous in the practice of the invention to be in compliance with Art. 53(c) EPC and Rule 28(b) and (c) EPC. Nothing herein is intended as a promise.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one” or “X or more”, where X is a number and understand to mean X or increases one by one of X, such as one or more or at least one member(s) or “X or more” of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In this description of the invention, reference is made to the accompanying drawings that form a part hereof, and in which are shown by way of illustration only of specific embodiments in which the invention may be practiced. It is to be understood that other embodiments may be utilized and structural or logical changes may be made without departing from the scope of the present invention. The description, therefore, is not to be taken in a limiting sense, and the scope of the present invention is defined by the appended claims.

It is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

To facilitate an understanding of the present invention, a number of terms and phrases are defined herein:

Unless specifically stated or obvious from context, as used herein, the term “about” is understood as within a range of normal tolerance in the art, for example within 2 standard deviations of the mean. About can be understood as within 50%, 45%, 40%, 35%, 30%, 25%, 20%, 15%, 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Unless specifically stated or obvious from context, as used herein, the term “or” is understood to be inclusive. Unless specifically stated or obvious from context, as used herein, the terms “a,” “an,” and “the” are understood to be singular or plural.

All gene name symbols refer to the gene as commonly known in the art. Gene symbols may be those refered to by the HUGO Gene Nomenclature Committee (HGNC). Any reference to the gene symbol is a reference made to the entire gene or variants of the gene. The HUGO Gene Nomenclature Committee is responsible for providing human gene naming guidelines and approving new, unique human gene names and symbols. All human gene names and symbols can be searched at www.genenames.org, the HGNC website, and the guidelines for their formation are available there (www.genenames.org/guidelines).

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease (e.g., a neoplasia, tumor, etc.).

By “alteration” is meant a change (increase or decrease) in the expression levels oractivity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “analog” is meant a molecule that is not identical, but has analogous functional or structural features. For example, a tumor specific neo-antigen polypeptide analog retains the biological activity of a corresponding naturally-occurring tumor specific neo-antigen polypeptide, while having certain biochemical modifications that enhance the analog's function relative to a naturally-occurring polypeptide. Such biochemical modifications could increase the analog's protease resistance, membrane permeability, or half-life, without altering, for example, ligand binding. An analog may include an unnatural amino acid.

“Combination therapy” is intended to embrace administration of therapeutic agents (e.g. neoantigenic peptides described herein) in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. For example, one combination of the present invention may comprise a pooled sample of neoantigenic peptides administered at the same or different times, or they can be formulated as a single, co-formulated pharmaceutical composition comprising the peptides. As another example, a combination of the present invention (e.g., a pooled sample of tumor specific neoantigens) may be formulated as separate pharmaceutical compositions that can be administered at the same or different time. As used herein, the term “simultaneously” is meant to refer to administration of one or more agents at the same time. For example, in certain embodiments, the neoantigenic peptides are administered simultaneously. Simultaneously includes administration contemporaneously, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. The phrase “combination” embraces groups of compounds or non-drug therapies useful as part of a combination therapy.

The term “neoantigen” or “neoantigenic” means a class of tumor antigens that arises from a tumor-specific mutation(s) which alters the amino acid sequence of genome encoded proteins.

By “neoplasia” is meant any disease that is caused by or results in inappropriately high levels of cell division, inappropriately low levels of apoptosis, or both. For example, cancer is an example of a neoplasia. Examples of cancers include, without limitation, leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The term “vaccine” is meant to refer in the present context to a pooled sample of tumor-specific neoantigenic peptides, for example at least two, at least three, at least four, at least five, or more neoantigenic peptides. A “vaccine” is to be understood as meaning a composition for generating immunity for the prophylaxis and/or treatment of diseases (e.g., neoplasia/tumor). Accordingly, vaccines are medicaments which comprise antigens and are intended to be used in humans or animals for generating specific defense and protective substance by vaccination. A “vaccine composition ” can include a pharmaceutically acceptable excipient, carrier or diluent.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” of pooled tumor specific neoantigens as recited herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethylsulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

By an isolated “polypeptide” or “peptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

As used herein, the terms “prevent,” “preventing,” “prevention,” “prophylactic treatment,” and the like, refer to reducing the probability of developing a disease or condition in a subject, who does not have, but is at risk of or susceptible to developing a disease or condition.

The term “prime/boost” or “prime/ boost dosing regimen” is meant to refer to the successive administrations of a vaccine or immunogenic or immunological compositions. The priming administration (priming) is the administration of a first vaccine or immunogenic or immunological composition type and may comprise one, two or more administrations. The boost administration is the second administration of a vaccine or immunogenic or immunological composition type and may comprise one, two or more administrations, and, for instance, may comprise or consist essentially of annual administrations. In certain embodiments, administration of the neoplasia vaccine or immunogenic composition is in a prime/boost dosing regimen.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “receptor” is to be understood as meaning a biological molecule or a molecule grouping capable of binding a ligand. A receptor may serve, to transmit information in a cell, a cell formation or an organism. The receptor comprises at least one receptor unit and frequently contains two or more receptor units, where each receptor unit may consist of a protein molecule, in particular a glycoprotein molecule. The receptor has a structure that complements the structure of a ligand and may complex the ligand as a binding partner. Signaling information may be transmitted by conformational changes of the receptor following binding with the ligand on the surface of a cell. According to the invention, a receptor may refer to particular proteins of MHC classes I and II capable of forming a receptor/ligand complex with a ligand, in particular a peptide or peptide fragment of suitable length.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The terms “treat,” “treated,” “treating,” “treatment,” and the like are meant to refer to reducing or ameliorating a disorder and/or symptoms associated therewith (e.g., a neoplasia or tumor). “Treating” may refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

The term “therapeutic effect” refers to some extent of relief of one or more of the symptoms of a disorder (e.g., a neoplasia or tumor) or its associated pathology. “Therapeutically effective amount” as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds of the invention employed in a pharmaceutical composition at levels lower than that required in order to achieve the desired therapeutic effect and gradually increase the dosage until the desired effect is achieved.

The terms “spacer” or “linker” as used in reference to a fusion protein refers to a peptide that joins the proteins comprising a fusion protein. Generally, a spacer has no specific biological activity other than to join or to preserve some minimum distance or other spatial relationship between the proteins or RNA sequences. However, in certain embodiments, the constituent amino acids of a spacer may be selected to influence some property of the molecule such as the folding, net charge, or hydrophobicity of the molecule.

Suitable linkers for use in an embodiment of the present invention are well known to those of skill in the art and include, but are not limited to, straight or branched-chain carbon linkers, heterocyclic carbon linkers, or peptide linkers. The linker is used to separate two neoantigenic peptides by a distance sufficient to ensure that, in a preferred embodiment, each neoantigenic peptide properly folds. Preferred peptide linker sequences adopt a flexible extended conformation and do not exhibit a propensity for developing an ordered secondary structure. Typical amino acids in flexible protein regions include Gly, Asn and Ser. Virtually any permutation of amino acid sequences containing Gly, Asn and Ser would be expected to satisfy the above criteria for a linker sequence. Other near neutral amino acids, such as Thr and Ala, also may be used in the linker sequence. Still other amino acid sequences that may be used as linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The therapy disclosed herein constitutes a new method for treating various types of cancer. The therapy described herein also provides a method of therapy for achieving clinical benefit without an unacceptable level of side effects.

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells, and are then presented to naïve or cytotoxic T-lymphocytes (CTLs). MHC class II proteins are present on dendritic cells, B-lymphocytes, macrophages and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself, and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

The peptide antigens attach themselves to the molecules of MHC class I by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines. The human leukocyte antigen (HLA) system is a gene complex encoding the major histocompatibility complex (MHC) proteins in humans.

By “proteins or molecules of the major histocompatibility complex (MHC)”, “MHC molecules”, “MHC proteins” or “HLA proteins” is thus meant proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells.

MHC molecules of class I consist of a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

MHC molecules of class II consist of an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

Subject specific HLA alleles or HLA genotype of a subject may be determined by any method known in the art. In preferred embodiments, HLA genotypes are determined by any method described in International Patent Application number PCT/US2014/068746, published Jun. 11, 2015 as WO2015085147. Briefly, the methods include determining polymorphic gene types that may comprise generating an alignment of reads extracted from a sequencing data set to a gene reference set comprising allele variants of the polymorphic gene, determining a first posterior probability or a posterior probability derived score for each allele variant in the alignment, identifying the allele variant with a maximum first posterior probability or posterior probability derived score as a first allele variant, identifying one or more overlapping reads that aligned with the first allele variant and one or more other allele variants, determining a second posterior probability or posterior probability derived score for the one or more other allele variants using a weighting factor, identifying a second allele variant by selecting the allele variant with a maximum second posterior probability or posterior probability derived score, the first and second allele variant defining the gene type for the polymorphic gene, and providing an output of the first and second allele variant.

As described herein, there is a large body of evidence in both animals and humans that mutated epitopes are effective in inducing an immune response and that cases of spontaneous tumor regression or long term survival correlate with CD8+ T-cell responses to mutated epitopes (Buckwalter and Srivastava PK. “It is the antigen(s), stupid” and other lessons from over a decade of vaccitherapy of human cancer. Seminars in immunology 20:296-300 (2008); Karanikas et al, High frequency of cytolytic T lymphocytes directed against a tumor-specific mutated antigen detectable with HLA tetramers in the blood of a lung carcinoma patient with long survival. Cancer Res. 61:3718-3724 (2001); Lennerz et al, The response of autologous T cells to a human melanoma is dominated by mutated neoantigens. Proc Natl Acad Sci USA.102:16013 (2005)) and that “immunoediting” can be tracked to alterations in expression of dominant mutated antigens in mice and man (Matsushita et al, Cancer exome analysis reveals a T-cell-dependent mechanism of cancer immunoediting Nature 482:400 (2012); DuPage et al, Expression of tumor-specific antigens underlies cancer immunoediting Nature 482:405 (2012); and Sampson et al, Immunologic escape after prolonged progression-free survival with epidermal growth factor receptor variant III peptide vaccination in patients with newly diagnosed glioblastoma J Clin Oncol. 28:4722-4729 (2010)).

Sequencing technology has revealed that each tumor contains multiple, patient-specific mutations that alter the protein coding content of a gene. Such mutations create altered proteins, ranging from single amino acid changes (caused by missense mutations) to addition of long regions of novel amino acid sequence due to frame shifts, read-through of termination codons or translation of intron regions (novel open reading frame mutations; neoORFs). These mutated proteins are valuable targets for the host's immune response to the tumor as, unlike native proteins, they are not subject to the immune-dampening effects of self-tolerance. Therefore, mutated proteins are more likely to be immunogenic and are also more specific for the tumor cells compared to normal cells of the patient.

### Improved HLA Epitope Prediction, Methods and Products for use Therein

Provided herein are methods and tools for improved HLA epitope prediction. These are of interest, for example, for use in the production of suitable neoantigen-comprising peptides as described herein below.

In one aspect, the present disclosure provides methods for generating an HLA-allele specific binding peptide sequence database. Such a database is very useful for predicting suitable HLA-binding peptides, identifying factors which play a role in HLA-peptide presentation and generating a more accurate prediction algorithm for identifying HLA-allele specific binding peptides. The methods comprise isolating and sequencing, for each HLA-allele, the HLA-binding peptides. In particular embodiments, the methods comprise providing a) a population of cells which expresses a single class I HLA allele or a single pair of class II HLA alleles (one α-chain and one β-chain); b) isolating the respective HLA-peptide complexes from said cells; c) isolating peptides from said HLA-peptide complexes; and d) sequencing said peptides. One of the advantages of the present method is the ability to identify a large number of HLA binding peptides which are specific for a particular HLA allele.

The method comprises providing a population of cells that expresses either a single class I HLA allele, a single pair of class II HLA alleles, or a single class I HLA allele and a single pair of class II HLA alleles. Suitable cell populations include, e.g., class I deficient cells lines in which a single HLA class I allele is expressed, class II deficient cell lines in which a single pair of HLA class II alleles are expressed, or class I and class II deficient cell lines in which a single HLA class I and/or single pair of class II alleles are expressed. As an exemplary embodiment, the class I deficient B cell line is B721.221. However, it is clear to a skilled person that other cell populations can be generated which are class I and/or class II deficient. An exemplary method for deleting/inactivating endogenous class I or class II genes includes, CRISPR-Cas9 mediated genome editing.

In preferred embodiments, the population of cells are professional antigen presenting cells such as macrophages, B cells and dendritic cells. Preferably, the cells are B cells or dendritic cells.

In preferred embodiments the cells are tumor cells or cells from a tumor cell line. In particular embodiments, the cells are cells isolated from a patient.

In preferred embodiments, the population of cells comprises at least 10⁷ cells.

In some embodiments, the population of cells are further modified, such as by increasing or decreasing the expression and/or activity of at least one gene. In preferred embodiments, the gene encodes a member of the immunoproteasome. The immunoproteasome is known to be involved in the processing of HLA class I binding peptides and includes the LMP2 (β1i), MECL-1 (β2i), and LMP7 (β5i) subunits. The immunoproteasome can also be induced by interferon-gamma. Accordingly, in some embodiments, the population of cells may be contacted with one or more cytokines, growth factors, or other proteins. Preferably, the cells are stimulated with inflammatory cytokines such as interferon-gamma, IL-1β, IL-6, and/or TNF-α. The population of cells may also be subjected to various environmental conditions, such as stress (heat stress, oxygen deprivation, glucose starvation, DNA damaging agents, etc.). In some embodiments the cells are contacted with one or more of a chemotherapy drug, radiation, targeted therapies, immunotherapy. The methods disclosed herein can therefore be used to study the effect of various genes or conditions on HLA peptide processing and presentation. In particular embodiments, the conditions used are selected so as to match the condition of the patient for which the population of HLA-peptides is to be identified.

Any HLA allele may be expressed in the cell population. Typically, it will be of interest to sequentially perform the methods provided herein for different HLA alleles, such that resulting datasets can be used in combination. In a preferred embodiment, the HLA allele is a class I HLA allele. In particular embodiments, the class I HLA allele is an HLA-A allele or an HLA-B allele. In a preferred embodiment, the HLA allele is a class II HLA allele. Sequences of class I and class II HLA alleles can be found in the IPD-IMGT/HLA Database. Exemplary HLA alleles include but are not limited to A*01:01, A*02:01, A*02:03, A*02:04, A*02:07, A*03:01, A*24:02, A*29:02, A*31:01, A*68:02, B*35:01, B*44:02, B*44:03, B*51:01, B*54:01 or B57:01 In particular embodiments, the HLA allele is selected so as to correspond to a genotype of interest. In a preferred embodiment, the HLA allele is a mutated HLA allele, which may be non-naturally occurring allele or a naturally occurring allele in an afflicted patient. The methods disclosed herein have the further advantage of identifying HLA binding peptides for HLA alleles associated with various disorders as well as alleles which are present at low frequency. Accordingly, in a preferred method the HLA allele is present at a frequency of less than 1% within a population, such as within the Caucasian population.

Vectors, promoters, etc for expression. In some embodiments, the nucleic acid sequence encoding the HLA allele further comprises a peptide tag which can be used to immunopurify the HLA-protein. Suitable tags are well-known in the art and include Myc, VSV, V5, His, HA, and FLAG tags.

The methods further comprise isolating HLA-peptide complexes from said cells. In preferred embodiments the complexes can be isolated using standard immunoprecipitation techniques known in the art with commercially available antibodies. Preferably, the cells are first lysed. HLA class I-peptide complexes can be isolated using HLA class I specific antibodies such as the W6/32 antibody, while HLA class II-peptide complexes can be isolated using HLA class II specific antibodies such as the M5/114.15.2 monoclonal antibody. In some embodiments, the single (or pair of) HLA alleles are expressed as a fusion protein with a peptide tag and the HLA-peptide complexes are isolated using binding molecules that recognize the peptide tags.

The methods further comprise isolating peptides from said HLA-peptide complexes and sequencing the peptides. The peptides are isolated from the complex by any method known to one of skill in the art, such as acid elution. While any sequencing method may be used, methods employing mass spectrometry, such as liquid chromatography-mass spectrometry (LC-MS or LC-MS/MS, or alternatively HPLC-MS or HPLC-MS/MS) are preferred. These sequencing methods are well-known to a skilled person and are reviewed in Medzihradszky K F and Chalkley R J. Mass Spectrom Rev. 2015 January-February; 34(1):43-63.

Typically, an HLA-allele specific binding peptide sequence database comprises at least 1000 different binding peptide sequences.

The methods disclosed herein may also be used to generate a database comprising the HLA-allele specific binding peptide sequences for more than one HLA-allele. In preferred embodiments, the methods comprise performing the steps a)-d) for at least two different HLA-alleles, preferably at least five, more preferably at least 10 different alleles.

In one aspect, the present disclosure provides a plurality of HLA-allele specific binding peptides, or the sequences thereof, which peptides correspond to the peptides which are presented by one specific HLA allele. More particularly, an HLA-allele specific binding peptide sequence database is provided obtained by carrying out the method according to the invention. In particular embodiments, combinations of pluralities of peptides, sets of sequences or databases is provided, represent HLA-allele specific peptides, sets of sequences or databases for different HLA alleles. The combination of databases is also referred to herein as a dataset. These combinations differentiate themselves over prior art datasets in that they represent HLA-specific peptides for each HLA-allele individually rather than combining HLA peptides obtained from a combination of HLA-alleles.

In one aspect, the present disclosure provides methods for generating a prediction algorithm for identifying HLA-allele specific binding peptides, which methods comprise training a neural network with one or more peptide sequence databases (i.e; combinations of databases). In particular embodiments, the methods involve training a machine with one or more peptide sequence databases generated with a method according to the invention. More particularly, the methods comprise training a neural network running on a machine with several peptide sequence databases. In the methods provided herein, the sequences are compared so as to identify prediction algorithms for a peptide to be presented by said HLA-allele.

Generating a prediction algorithm by training a machine is a well-known technique. The most important in the training of the machine is the quality of the database used for the training. Typically, the machine combines one or more linear models, support vector machines, decision trees and/or a neural network.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory.

In a preferred embodiment, the methods involve generating models based on predictive variables. In particular embodiments, only peptide-intrinsic features are used as variables (such as sequence, amino acid properties, peptide characteristics). In alternative embodiments, the models also incorporate extrinsic features such as expression and cleavage information. In particular embodiments, the variables used to train the machine comprise one or more predictive variables selected from the group consisting of peptide sequence, amino acid physical properties, peptide physical properties, protein stability, protein translation rate, protein degradation rate, translational efficiencies from ribosomal profiling, protein cleavability, protein localization, motifs of host protein that facilitate TAP transport, whether host protein is subject to autophagy, motifs that favor ribosomal stalling (polyproline stretches) and protein features that favor NMD (long 3′ UTR, stop codon >50 nt upstream of last exon:exon junction). In particular embodiments, at least two of these features are used. In further embodiments, at least 3, 4, 5, 6, 7, 8, 9 or all ten of these features are used. In a preferred embodiment, the variables used to train the machine comprise the expression level of the source protein of a peptide within a cell. In a preferred embodiment, the variables used to train the machine comprise expression level of the source protein of a peptide within a cell, peptide sequence, amino acid physical properties, peptide physical properties, expression level of the source protein of a peptide within a cell, Protein stability, protein translation rate, protein degradation rate, translational efficiencies from ribosomal profiling, protein cleavability, protein localization, motifs of host protein that facilitate TAP transport, host protein is subject to autophagy, motifs that favor ribosomal stalling (polyproline stretches), protein features that favor NMD (long 3′ UTR, stop codon >50 nt upstream of last exon:exon junction and peptide cleav ability.

In one aspect, the present disclosure provides methods for identifying HLA-allele specific binding peptides, which method comprises analyzing the sequence of a peptide with a machine which has been trained with a peptide sequence database obtained by carrying out the method according to the invention for said HLA-allele. In a preferred embodiment, the method comprises using information on the expression level of the source protein of the peptide within the cell as a variable. In further embodiments, the method comprises determining the expression level of the source protein of the peptide within a cell and using the source protein expression as one of the predictive variables used by the machine. Typically, the expression level is determined by measuring the amount of source protein or the amount of RNA encoding said source protein. It is demonstrated herein that the methods provided herein allow a more effective prediction of HLA-binding peptides than methods of the prior art, with fewer false positives. This is important as the number of immunogenic peptides that can practically be generated in the context of an immune therapy is limited. In particular embodiments, the methods are used to determine an effective neoantigen vaccine. In this context, it is of interest to determine which peptides forming neoantigens are likely to bind to a subject's HLA so as to effectively function as immunogenic peptides.

### Production of Tumor Specific Neoantigens

One of the critical barriers to developing curative and tumor-specific immunotherapy is the identification and selection of highly specific and restricted tumor antigens to avoid autoimmunity. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized neoantigens, and producing neoantigens for use in a vaccine or immunogenic composition. These problems may be addressed by:
identifying mutations in neoplasias/tumors which are present at the DNA level in tumor but not in matched germline samples from a high proportion of subjects having cancer;
analyzing the identified mutations with one or more peptide-MHC binding prediction algorithms to generate a plurality of neoantigen T cell epitopes that are expressed within the neoplasia/tumor and that bind to a high proportion of patient HLA alleles; and
synthesizing the plurality of neoantigenic peptides selected from the sets of all neoantigen peptides and predicted binding peptides for use in a cancer vaccine or immunogenic composition suitable for treating a high proportion of subjects having cancer.

For example, translating sequencing information into a therapeutic vaccine may include:

(1) Prediction of mutated peptides that can bind to HLA molecules of a high proportion of individuals. Efficiently choosing which particular mutations to utilize as immunogen requires the ability to predict which mutated peptides would efficiently bind to a high proportion of patient's HLA alleles. Recently, neural network based learning approaches with validated binding and non-binding peptides have advanced the accuracy of prediction algorithms for the major HLA-A and -B alleles.

(2) Formulating the drug as a multi-epitope vaccine of long peptides. Targeting as many mutated epitopes as practically possible takes advantage of the enormous capacity of the immune system, prevents the opportunity for immunological escape by down-modulation of a particular immune targeted gene product, and compensates for the known inaccuracy of epitope prediction approaches. Synthetic peptides provide a particularly useful means to prepare multiple immunogens efficiently and to rapidly translate identification of mutant epitopes to an effective vaccine. Peptides can be readily synthesized chemically and easily purified utilizing reagents free of contaminating bacteria or animal substances. The small size allows a clear focus on the mutated region of the protein and also reduces irrelevant antigenic competition from other components (unmutated protein or viral vector antigens).

(3) Combination with a strong vaccine adjuvant. Effective vaccines require a strong adjuvant to initiate an immune response. As described below, poly-ICLC, an agonist of TLR3 and the RNA helicase-domains of MDA5 and RIGS, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NYES0-1 peptide vaccine (in addition to Montanide) showed induction of CD4+ and CD8+ T cell, as well as antibody responses to the peptide in a recent phase 1 study. At the same time, polyICLC has been extensively tested in more than 25 clinical trials to date and exhibited a relatively benign toxicity profile.

The application provides improved methods of prediction of peptides, such as mutated peptides, that can bind to HLA molecules of a high proportion of individuals. In particular embodiments, the application provides methods of identifying from a given set of neo-antigen comprising peptides the most suitable peptides for preparing an immunogenic composition for a subject, said method comprising selecting from set given set of peptides the plurality of peptides capable of binding an HLA protein of the subject, wherein said ability to bind an HLA protein is determined by analyzing the sequence of peptides with a machine which has been trained with peptide sequence databases corresponding to the specific HLA-binding peptides for each of the HLA-alleles of said subject. More particularly, the application provides methods of identifying from a given set of neo-antigen comprising peptides the most suitable peptides for preparing an immunogenic composition for a subject, said method comprising selecting from set given set of peptides the plurality of peptides determined as capable of binding an HLA protein of the subject, ability to bind an HLA protein is determined by analyzing the sequence of peptides with a machine which has been trained with a peptide sequence database obtained by carrying out the methods described herein above. Thus, in particular embodiments, the application provides methods of identifying a plurality of subject-specific peptides for preparing a subject-specific immunogenic composition, wherein the subject has a tumor and the subject-specific peptides are specific to the subject and the subject's tumor, said method comprising:
whole genome or whole exome nucleic acid sequencing of a sample of the subject's tumor and a non-tumor sample of the subject;
determining based on the whole genome or whole exome nucleic acid sequencing:
   non-silent mutations present in the genome of cancer cells of the subject but not in normal tissue from the subject, and
   the HLA genotype of the subject,
wherein the non-silent mutations comprise a point, splice-site, frameshift, read-through or gene-fusion mutation; and
selecting from the identified non-silent mutations the plurality of subject-specific peptides, each having a different tumor neo-epitope that is an epitope specific to the tumor of the subject and each being identified as capable of binding an HLA protein of the subject, as determined by analyzing the sequence of peptides derived from the non-silent mutations in the methods for predicting HLA binding described herein.

In particular embodiments, the methods are used to determine whether or not a peptide will bind to an HLA protein. In further embodiments, the methods provide a predictive score indicative of binding an HLA protein of the subject,

Thus, in particular embodiments, the application provides methods of identifying a plurality of subject-specific peptides for preparing a subject-specific immunogenic composition, said method comprising selecting a plurality of subject-specific peptides, each having a different tumor neo-epitope that is an epitope specific to the tumor of the subject and each having a predictive score indicative of binding an HLA protein of the subject, wherein said predictive score is determined by analyzing the sequence of peptides derived from the non-silent mutations by carrying out the method of predicting HLA-binding described herein.

In particular embodiments, the cell used in the method for determining HLA binding as described herein is an antigen-presenting cell.

In a further aspect, the invention provides methods for identifying tumor neonatigen-comprising peptides, wherein the methods comprise identifying for a given HLA allele, the peptides binding said HLA allele in a tumor cell from a tumor of a patient.

The application further provides novel neoantigenic peptides identified by the methods provided herein. Accordingly, provided herein are immunogenic compositions comprising a peptide having a sequence selected from XLXX₄XX₆X₇XX₉, wherein one or more of X₄ is E or D, X₆ is L, V, or I, X₇ is I, V, or A, and X₉ is L or V, and wherein X is any amino acid; XLXDXXX₇XX₉, wherein one or more of X₇ is L and X₉ is Y or F, and wherein X is any amino acid; XX₂X₃X₄XXXXY, wherein one or more of X₂ is T, S, or L, X₃ is D or E and X₇ is I, V, or A, and wherein X is any amino acid; XLXXXX₆XXX₉, wherein one or more of X₆ is L or V and X₉ is V or L, and wherein X is any amino acid; XLXX₄XX₆XXX₉, wherein one or more of X₄ is E or D, X₆ is L or V and X₉ is V or L, and wherein X is any amino acid; XLDXXXXXX₉, wherein X₉ is L or V, and wherein X is any amino acid; XXXXXXLXX₉, wherein one or more of X₂ is L or V and X₉ is K, Y or R, and wherein X is any amino acid; X₁X₂XXXXXXR, wherein one or more of X₁ is R or A and X₂ is V or L, and wherein X is any amino acid; EX₂XXXXXXX₉, wherein one or more of X₂ is V, T, or A and X₉ is V or L, and wherein X is any amino acid;

XX₂XRXXXXX₉, wherein one or more of X₂ is P or A and X₉ is Y, F, or L, and wherein X is any amino acid; X₁EXXLXXXX₉, wherein one or more of X₁ is A or E and X₉ is F, W, or L, and wherein X is any amino acid; X₁EXXLXLXX₉, wherein one or more of X₁ is A or E and X₉ is F, W, or L, and wherein X is any amino acid; DX₂XXXXXXX₉, wherein one or more of X₂ is P or A and X₉ is I, V, or L, and wherein X is any amino acid; and X₁YXXXXXXX₉, wherein one or more of X₁ is M, W, or V and X₉ is F or L, and wherein X is any amino acid.

The present invention is based, at least in part, on the ability to present the immune system of the patient with a pool of tumor specific neoantigens. One of skill in the art from this disclosure and the knowledge in the art will appreciate that there are a variety of ways in which to produce such tumor specific neoantigens. In general, such tumor specific neoantigens may be produced either in vitro or in vivo. Tumor specific neoantigens may be produced in vitro as peptides or polypeptides, which may then be formulated into a neoplasia vaccine or immunogenic composition and administered to a subject. As described in further detail herein, such in vitro production may occur by a variety of methods known to one of skill in the art such as, for example, peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, tumor specific neoantigens may be produced in vivo by introducing molecules (e.g., DNA, RNA, viral expression systems, and the like) that encode tumor specific neoantigens into a subject, whereupon the encoded tumor specific neoantigens are expressed. The methods of in vitro and in vivo production of neoantigens is also further described herein as it relates to pharmaceutical compositions and methods of delivery of the therapy.

In certain embodiments the present invention includes modified neoantigenic peptides. As used herein in reference to neoantigenic peptides, the terms “modified”, “modification” and the like refer to one or more changes that enhance a desired property of the neoantigenic peptide, where the change does not alter the primary amino acid sequence of the neoantigenic peptide. “Modification” includes a covalent chemical modification that does not alter the primary amino acid sequence of the neoantigenic peptide itself. Such desired properties include, for example, prolonging the in vivo half-life, increasing the stability, reducing the clearance, altering the immunogenicity or allergenicity, enabling the raising of particular antibodies, cellular targeting, antigen uptake, antigen processing, MHC affinity, MHC stability, or antigen presentation. Changes to a neoantigenic peptide that may be carried out include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

The clinical effectiveness of protein therapeutics is often limited by short plasma half-life and susceptibility to protease degradation. Studies of various therapeutic proteins (e.g., filgrastim) have shown that such difficulties may be overcome by various modifications, including conjugating or linking the polypeptide sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide sequence are generally soluble in water at room temperature, and have the general formula R(0-CH₂—CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, but certain embodiments have a molecular weight between 500 and 20,000 while other embodiments have a molecular weight between 4,000 and 10,000.

The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to a polypeptide of the present disclosure via a terminal reactive group (a “spacer”). The spacer is, for example, a terminal reactive group which mediates a bond between the free amino or carboxyl groups of one or more of the polypeptide sequences and polyethylene glycol. The PEG having the spacer which may be bound to the free amino group includes N-hydroxysuccinylimide polyethylene glycol which may be prepared by activating succinic acid ester of polyethylene glycol with N-hydroxy succinylimide. Another activated polyethylene glycol which may be bound to a free amino group is 2,4-bis(0-methoxypolyethyleneglycol)-6-chloro-s-triazine which may be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4: 1 to 30: 1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

The present disclosure also contemplates the use of PEG Mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

For purposes of the present disclosure, “glycosylation” is meant to broadly refer to the enzymatic process that attaches glycans to proteins, lipids or other organic molecules. The use of the term “glycosylation” in conjunction with the present disclosure is generally intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins involving a change in the nature and proportions of the various carbohydrate moieties present. Glycosylation can dramatically affect the physical properties of proteins and can also be important in protein stability, secretion, and subcellular localization. Proper glycosylation can be essential for biological activity. In fact, some genes from eucaryotic organisms, when expressed in bacteria (e.g., E. coli) which lack cellular processes for glycosylating proteins, yield proteins that are recovered with little or no activity by virtue of their lack of glycosylation.

Addition of glycosylation sites can be accomplished by altering the amino acid sequence. The alteration to the polypeptide may be made, for example, by the addition of, or substitution by, one or more serine or threonine residues (for O-linked glycosylation sites) or asparagine residues (for N-linked glycosylation sites). The structures of N-linked and O-linked oligosaccharides and the sugar residues found in each type may be different. One type of sugar that is commonly found on both is N-acetylneuraminic acid (hereafter referred to as sialic acid). Sialic acid is usually the terminal residue of both N-linked and O-linked oligosaccharides and, by virtue of its negative charge, may confer acidic properties to the glycoprotein. A particular embodiment of the present disclosure comprises the generation and use of N-glycosylation variants.

The polypeptide sequences of the present disclosure may optionally be altered through changes at the DNA level, particularly by mutating the DNA encoding the polypeptide at preselected bases such that codons are generated that will translate into the desired amino acids. Another means of increasing the number of carbohydrate moieties on the polypeptide is by chemical or enzymatic coupling of glycosides to the polypeptide.

Removal of carbohydrates may be accomplished chemically or enzymatically, or by substitution of codons encoding amino acid residues that are glycosylated. Chemical deglycosylation techniques are known, and enzymatic cleavage of carbohydrate moieties on polypeptides can be achieved by the use of a variety of endo- and exo-glycosidases.

Dihydrofolate reductase (DHFR)-deficient Chinese Hamster Ovary (CHO) cells are a commonly used host cell for the production of recombinant glycoproteins. These cells do not express the enzyme beta-galactoside alpha-2,6-sialyltransferase and therefore do not add sialic acid in the alpha-2,6 linkage to N-linked oligosaccharides of glycoproteins produced in these cells.

The present disclosure also contemplates the use of polysialylation, the conjugation of peptides and proteins to the naturally occurring, biodegradable a-(2→8) linked polysialic acid (“PSA”) in order to improve their stability and in vivo pharmacokinetics. PSA is a biodegradable, non-toxic natural polymer that is highly hydrophilic, giving it a high apparent molecular weight in the blood which increases its serum half-life. In addition, polysialylation of a range of peptide and protein therapeutics has led to markedly reduced proteolysis, retention of activity in vivo activity, and reduction in immunogenicity and antigenicity (see, e.g., G. Gregoriadis et al., Int. J. Pharmaceutics 300(1-2): 125-30). As with modifications with other conjugates (e.g., PEG), various techniques for site-specific polysialylation are available (see, e.g., T. Lindhout et al., PNAS 108(18)7397-7402 (2011)).

Additional suitable components and molecules for conjugation include, for example, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemaglutinin, influenza virus nucleoprotein; Keyhole Limpet Hemocyanin (KLH); and hepatitis B virus core protein and surface antigen; or any combination of the foregoing.

Fusion of albumin to one or more polypeptides of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Transformation is used broadly herein to refer to the genetic alteration of a cell resulting from the direct uptake, incorporation and expression of exogenous genetic material (exogenous DNA) from its surroundings and taken up through the cell membrane(s). Transformation occurs naturally in some species of bacteria, but it can also be effected by artificial means in other cells.

Furthermore, albumin itself may be modified to extend its circulating half-life. Fusion of the modified albumin to one or more Polypeptides can be attained by the genetic manipulation techniques described above or by chemical conjugation; the resulting fusion molecule has a half-life that exceeds that of fusions with non-modified albumin. (See WO2011/051489).

Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics. For example, insulin determir (LEVEMIR), an approved product for diabetes, comprises a myristyl chain conjugated to a genetically-modified insulin, resulting in a long-acting insulin analog.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule.

A conjugate modification may result in a polypeptide sequence that retains activity with an additional or complementary function or activity of the second molecule. For example, a polypeptide sequence may be conjugated to a molecule, e.g., to facilitate solubility, storage, in vivo or shelf half-life or stability, reduction in immunogenicity, delayed or controlled release in vivo, etc. Other functions or activities include a conjugate that reduces toxicity relative to an unconjugated polypeptide sequence, a conjugate that targets a type of cell or organ more efficiently than an unconjugated polypeptide sequence, or a drug to further counter the causes or effects associated with a disorder or disease as set forth herein (e.g., diabetes).

A Polypeptide may also be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Additional candidate components and molecules for conjugation include those suitable for isolation or purification. Particular non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes.

Purification methods such as cation exchange chromatography may be used to separate conjugates by charge difference, which effectively separates conjugates into their various molecular weights. For example, the cation exchange column can be loaded and then washed with −20 mM sodium acetate, pH −4, and then eluted with a linear (0 M to 0.5 M) NaCl gradient buffered at a pH from about 3 to 5.5, e.g., at pH −4.5. The content of the fractions obtained by cation exchange chromatography may be identified by molecular weight using conventional methods, for example, mass spectroscopy, SDS-PAGE, or other known methods for separating molecular entities by molecular weight.

In certain embodiments, the amino- or carboxyl-terminus of a polypeptide sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration.

Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamic properties of the biopharmaceutical as compared to traditional Fc-fusion conjugates.

The present disclosure contemplates the use of other modifications, currently known or developed in the future, of the Polypeptides to improve one or more properties. One such method for prolonging the circulation half-life, increasing the stability, reducing the clearance, or altering the immunogenicity or allergenicity of a polypeptide of the present disclosure involves modification of the polypeptide sequences by hesylation, which utilizes hydroxyethyl starch derivatives linked to other molecules in order to modify the molecule's characteristics. Various aspects of hesylation are described in, for example, U.S. Patent Appin. Nos. 2007/0134197 and 2006/0258607.

### In Vitro Peptide/Polypeptide Synthesis

Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. The nucleotide and protein, polypeptide and peptide sequences corresponding to various genes have been previously disclosed, and may be found at computerized databases known to those of ordinary skill in the art. One such database is the National Center for Biotechnology Information's Genbank and GenPept databases located at the National Institutes of Health website. The coding regions for known genes may be amplified and/or expressed using the techniques disclosed herein or as would be known to those of ordinary skill in the art. Alternatively, various commercial preparations of proteins, polypeptides and peptides are known to those of skill in the art.

Peptides can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963). In certain embodiments, neoantigenic peptides are prepared by (1) parallel solid-phase synthesis on multi-channel instruments using uniform synthesis and cleavage conditions; (2) purification over a RP-HPLC column with column stripping; and re-washing, but not replacement, between peptides; followed by (3) analysis with a limited set of the most informative assays. The Good Manufacturing Practices (GMP) footprint can be defined around the set of peptides for an individual patient, thus requiring suite changeover procedures only between syntheses of peptides for different patients.

Alternatively, a nucleic acid (e.g., a polynucleotide) encoding a neoantigenic peptide of the invention may be used to produce the neoantigenic peptide in vitro. The polynucleotide may be, e.g., DNA, cDNA, PNA, CNA, RNA, either single- and/or double-stranded, or native or stabilized forms of polynucleotides, such as e.g. polynucleotides with a phosphorothiate backbone, or combinations thereof and it may or may not contain introns so long as it codes for the peptide. In one embodiment in vitro translation is used to produce the peptide. Many exemplary systems exist that one skilled in the art could utilize (e.g., Retic Lysate IVT Kit, Life Technologies, Waltham, Mass.).

An expression vector capable of expressing a polypeptide can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

Expression vectors comprising the isolated polynucleotides, as well as host cells containing the expression vectors, are also contemplated. The neoantigenic peptides may be provided in the form of RNA or cDNA molecules encoding the desired neoantigenic peptides. One or more neoantigenic peptides of the invention may be encoded by a single expression vector.

The term “polynucleotide encoding a polypeptide” encompasses a polynucleotide which includes only coding sequences for the polypeptide as well as a polynucleotide which includes additional coding and/or non-coding sequences. Polynucleotides can be in the form of RNA or in the form of DNA. DNA includes cDNA, genomic DNA, and synthetic DNA; and can be double-stranded or single-stranded, and if single stranded can be the coding strand or non-coding (anti-sense) strand.

In embodiments, the polynucleotides may comprise the coding sequence for the tumor specific neoantigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In embodiments, the polynucleotides can comprise the coding sequence for the tumor specific neoantigenic peptide fused in the same reading frame to a marker sequence that allows, for example, for purification of the encoded polypeptide, which may then be incorporated into the personalized neoplasia vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neoantigenic peptides fused in the same reading frame to create a single concatamerized neoantigenic peptide construct capable of producing multiple neoantigenic peptides.

In certain embodiments, isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigenic peptide of the present invention, can be provided.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

The isolated tumor specific neoantigenic peptides described herein can be produced in vitro (e.g., in the laboratory) by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585.

In embodiments, a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Recombinant expression vectors may be used to amplify and express DNA encoding the tumor specific neoantigenic peptides. Recombinant expression vectors are replicable DNA constructs which have synthetic or cDNA-derived DNA fragments encoding a tumor specific neoantigenic peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a cancer stem cell protein-Fc composition. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

### In Vivo Peptide/Polypeptide Synthesis

The present invention also contemplates the use of nucleic acid molecules as vehicles for delivering neoantigenic peptides/polypeptides to the subject in need thereof, in vivo, in the form of, e.g., DNA/RNA vaccines (see, e.g., WO2012/159643, and WO2012/159754, hereby incorporated by reference in their entirety).

In one embodiment neoantigens may be administered to a patient in need thereof by use of a plasmid. These are plasmids which usually consist of a strong viral promoter to drive the in vivo transcription and translation of the gene (or complementary DNA) of interest (Mor, et al., (1995), The Journal of Immunology 155 (4): 2039-2046). Intron A may sometimes be included to improve mRNA stability and hence increase protein expression (Leitner et al. (1997), The Journal of Immunology 159 (12): 6112-6119). Plasmids also include a strong polyadenylation/transcriptional termination signal, such as bovine growth hormone or rabbit beta-globulin polyadenylation sequences (Alarcon et al., (1999), Adv. Parasitol. Advances in Parasitology 42: 343-410; Robinson et al., (2000). Adv. Virus Res. Advances in Virus Research 55: 1-74; Bohmet al., (1996). Journal of Immunological Methods 193 (1): 29-40.). Multicistronic vectors are sometimes constructed to express more than one immunogen, or to express an immunogen and an immunostimulatory protein (Lewis et al., (1999). Advances in Virus Research (Academic Press) 54: 129-88).

Because the plasmid is the “vehicle” from which the immunogen is expressed, optimising vector design for maximal protein expression is essential (Lewis et al., (1999). Advances in Virus Research (Academic Press) 54: 129-88). One way of enhancing protein expression is by optimising the codon usage of pathogenic mRNAs for eukaryotic cells. Another consideration is the choice of promoter. Such promoters may be the SV40 promoter or Rous Sarcoma Virus (RSV).

Plasmids may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibres with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected(Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Ex Vivo Cytosolic Delivery of Functional Macromolecules to Immune Cells, Sharei et al, PLOS ONE | DOI:10.1371/journal.pone.0118803 Apr. 13, 2015).

The method of delivery determines the dose of DNA required to raise an effective immune response. Saline injections require variable amounts of DNA, from 10 μg-1 mg, whereas gene gun deliveries require 100 to 1000 times less DNA than intramuscular saline injection to raise an effective immune response. Generally, 0.2 μg-20 μg are required, although quantities as low as 16 ng have been reported. These quantities vary from species to species, with mice, for example, requiring approximately 10 times less DNA than primates. Saline injections require more DNA because the DNA is delivered to the extracellular spaces of the target tissue (normally muscle), where it has to overcome physical barriers (such as the basal lamina and large amounts of connective tissue, to mention a few) before it is taken up by the cells, while gene gun deliveries bombard DNA directly into the cells, resulting in less “wastage” (See e.g., Sedegah et al., (1994). Proceedings of the National Academy of Sciences of the United States of America 91 (21): 9866-9870; Daheshiaet al., (1997). The Journal of Immunology 159 (4): 1945-1952; Chen et al., (1998). The Journal of Immunology 160 (5): 2425-2432; Sizemore (1995) Science 270 (5234): 299-302; Fynan et al., (1993) Proc. Natl. Acad. Sci. U.S.A. 90 (24): 11478-82).

In one embodiment, a neoplasia vaccine or immunogenic composition may include separate DNA plasmids encoding, for example, one or more neoantigenic peptides/polypeptides as identified in according to the invention. As discussed herein, the exact choice of expression vectors can depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection.

One or more neoantigenic peptides of the invention may be encoded and expressed in vivo using a viral based system (e.g., an adenovirus system, an adeno associated virus (AAV) vector, a poxvirus, or a lentivirus). In one embodiment, the neoplasia vaccine or immunogenic composition may include a viral based vector for use in a human patient in need thereof, such as, for example, an adenovirus (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207(2):240-7, hereby incorporated by reference in its entirety). Plasmids that can be used for adeno associated virus, adenovirus, and lentivirus delivery have been described previously (see e.g., U.S. Pat. Nos. 6,955,808 and 6,943,019, and U.S. Patent application No. 20080254008, hereby incorporated by reference).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

Among vectors that may be used in the practice of the invention, integration in the host genome of a cell is possible with retrovirus gene transfer methods, often resulting in long term expression of the inserted transgene. In a preferred embodiment the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the invention is a minimal non-primate lentiviral vector, such as a lentiviral vector based on the equine infectious anemia virus (EIAV) (see, e.g., Balagaan, (2006) J Gene Med; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Accordingly, the invention contemplates amongst vector(s) useful in the practice of the invention: viral vectors, including retroviral vectors and lentiviral vectors.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for delivery to the Brain, see, e.g., US Patent Publication Nos. US20110293571; US20040013648, US20070025970, US20090111106 and U.S. Pat. No. 7,259,015. In another embodiment lentiviral vectors are used to deliver vectors to the brain of those being treated for a disease.

As to lentivirus vector systems useful in the practice of the invention, mention is made of U.S. Pat. Nos. 6,428,953, 6,165,782, 6,013,516, 5,994,136, 6,312,682, and 7,198,784, and documents cited therein.

In an embodiment herein the delivery is via an lentivirus. Zou et al. administered about 10 μl of a recombinant lentivirus having a titer of 1×10⁹ transducing units (TU)/ml by an intrathecal catheter. These sort of dosages can be adapted or extrapolated to use of a retroviral or lentiviral vector in the present invention. For transduction in tissues such as the brain, it is necessary to use very small volumes, so the viral preparation is concentrated by ultracentrifugation. The resulting preparation should have at least 10⁸ TU/ml, preferably from 10⁸ to 10⁹ TU/ml, more preferably at least 10⁹ TU/ml. Other methods of concentration such as ultrafiltration or binding to and elution from a matrix may be used.

In other embodiments the amount of lentivirus administered may be 1×10⁵ or about 1×10⁵ plaque forming units (PFU), 5×10⁵ or about 5×10⁵ PFU, 1×10⁶ or about 1×10⁶ PFU, 5×10⁶ or about 5×10⁶ PFU, 1×10⁷ or about 1×10⁷ PFU, 5×10⁷ or about 5×10⁷ PFU, 1×10⁸ or about 1×10⁸ PFU, 5×10⁸ or about 5×10⁸ PFU, 1×10⁹ or about 1×10⁹ PFU, 5×10⁹ or about 5×10⁹ PFU, 1×10¹⁰ or about 1×10¹⁰ PFU or 5×10¹⁰ or about 5×10¹⁰ PFU as total single dosage for an average human of 75 kg or adjusted for the weight and size and species of the subject. One of skill in the art can determine suitable dosage. Suitable dosages for a virus can be determined empirically.

Also useful in the practice of the invention is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the invention, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4, E1 and E3, and E1 and E4 can be used in accordance with the present invention. Furthermore, “gutless” adenovirus vectors, in which all viral genes are deleted, can also be used in accordance with the present invention. Such vectors require a helper virus for their replication and require a special human 293 cell line expressing both E1a and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present invention, and can even be used for co-delivery of a large number of heterologous inserts/genes.

In an embodiment herein the delivery is via an adenovirus, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×10⁹ particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1×10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4×10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element. For ubiquitous expression, the following promoters can be used: CMV, CAG, CBh, PGK, SV40, Ferritin heavy or light chains, etc. For brain expression, the following promoters can be used: Synapsinl for all neurons, CaMKIIalpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA).

With regard to AAV vectors useful in the practice of the invention, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein.

As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. The above promoters and vectors are preferred individually.

In an embodiment herein, the delivery is via an AAV. A therapeutically effective dosage for in vivo delivery of the AAV to a human is believed to be in the range of from about 20 to about 50 ml of saline solution containing from about 1×10¹° to about 1×10⁵° functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. In a preferred embodiment, AAV is used with a titer of about 2×10¹³ viral genomes/milliliter, and each of the striatal hemispheres of a mouse receives one 500 nanoliter injection. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In another embodiment effectively activating a cellular immune response for a neoplasia vaccine or immunogenic composition can be achieved by expressing the relevant neoantigens in a vaccine or immunogenic composition in a non-pathogenic microorganism. Well-known examples of such microorganisms are Mycobacterium bovis BCG, Salmonella and Pseudomona (See, U.S. Pat. No. 6,991,797, hereby incorporated by reference in its entirety).

In another embodiment a Poxvirus is used in the neoplasia vaccine or immunogenic composition. These include orthopoxvirus, avipox, vaccinia, MVA, NYVAC, canarypox, ALVAC, fowlpox, TROVAC, etc. (see e.g., Verardiet al., Hum Vaccin Immunother. 2012 July; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels.

Information concerning poxviruses that may be used in the practice of the invention, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature, such as:
U.S. Pat. Nos. 4,603,112, 4,769,330, 5,110,587, 5,174,993, 5,364,773, 5,762,938, 5,494,807, 5,766,597, 7,767,449, 6,780,407, 6,537,594, 6,265,189, 6,214,353, 6,130,066, 6,004,777, 5,990,091, 5,942,235, 5,833,975, 5,766,597, 5,756,101, 7,045,313, 6,780,417, 8,470,598, 8,372,622, 8,268,329, 8,268,325, 8,236,560, 8,163,293, 7,964,398, 7,964,396, 7,964,395, 7,939,086, 7,923,017, 7,897,156, 7,892,533, 7,628,980, 7,459,270, 7,445,924, 7,384,644, 7,335,364, 7,189,536, 7,097,842, 6,913,752, 6,761,893, 6,682,743, 5,770,212, 5,766,882, and 5,989,562, and
Panicali, D. Proc. Natl. Acad. Sci. 1982; 79; 4927-493, Panicali D. Proc. Natl. Acad. Sci. 1983; 80(17): 5364-8, Mackett, M. Proc. Natl. Acad. Sci. 1982; 79: 7415-7419, Smith G L. Proc. Natl. Acad. Sci. 1983; 80(23): 7155-9, Smith G L. Nature 1983; 302: 490-5, Sullivan V J. Gen. Vir. 1987; 68: 2587-98, Perkus M Journal of Leukocyte Biology 1995; 58:1-13, Yilma T D. Vaccine 1989; 7: 484-485, Brochier B. Nature 1991; 354: 520-22, Wiktor, T J. Proc. Natl Acd. Sci. 1984; 81: 7194-8, Rupprecht, C E. Proc. Natl Acd. Sci. 1986; 83: 7947-50, Poulet, H Vaccine 2007; 25(July): 5606-12, Weyer J. Vaccine 2009; 27(November): 7198-201, Buller, R M Nature 1985; 317(6040): 813-5, Buller R M. J. Virol. 1988; 62(3):866-74, Flexner, C. Nature 1987; 330(6145): 259-62, Shida, H. J. Virol. 1988; 62(12): 4474-80, Kotwal, G J. J. Virol. 1989; 63(2): 600-6, Child, S J. Virology 1990; 174(2): 625-9, Mayr A. Zentralbl Bakteriol 1978; 167(5,6): 375-9, Antoine G. Virology. 1998; 244(2): 365-96, Wyatt, L S. Virology 1998; 251(2): 334-42, Sancho, MC. J. Virol. 2002; 76(16); 8313-34, Gallego-Gomez, J C. J. Virol. 2003; 77(19); 10606-22), Goebel S J. Virology 1990; (a,b) 179: 247-66, Tartaglia, J. Virol. 1992; 188(1): 217-32, Najera J L. J. Virol. 2006; 80(12): 6033-47, Najera, J L. J. Virol. 2006; 80: 6033-6047, Gomez, C E. J. Gen. Virol. 2007; 88: 2473-78, Mooij, P. Jour. Of Virol. 2008; 82: 2975-2988, Gomez, C E. Curr. Gene Ther. 2011; 11: 189-217, Cox, W. Virology 1993; 195: 845-50, Perkus, M. Jour. Of Leukocyte Biology 1995; 58: 1-13, Blanchard T J. J Gen Virology 1998; 79(5): 1159-67, Amara R. Science 2001; 292: 69-74, Hel, Z., J. Immunol. 2001; 167: 7180-9, Gherardi M M. J. Virol. 2003; 77: 7048-57, Didierlaurent, A. Vaccine 2004; 22: 3395-3403, Bissht H. Proc. Nat. Aca. Sci. 2004; 101: 6641-46, McCurdy L H. Clin. Inf. Dis 2004; 38: 1749-53, Earl P L. Nature 2004; 428: 182-85, Chen Z. J. Virol. 2005; 79: 2678-2688, Najera J L. J. Virol. 2006; 80(12): 6033-47, Nam J H. Acta. Virol. 2007; 51: 125-30, Antonis A F. Vaccine 2007; 25: 4818-4827,B Weyer J. Vaccine 2007; 25: 4213-22, Ferrier-Rembert A. Vaccine 2008; 26(14): 1794-804, Corbett M. Proc. Natl. Acad. Sci. 2008; 105(6): 2046-51, Kaufman H L., J. Clin. Oncol. 2004; 22: 2122-32, Amato, R J. Clin. Cancer Res. 2008; 14(22): 7504-10, Dreicer R. Invest New Drugs 2009; 27(4): 379-86, Kantoff P W. J. Clin. Oncol. 2010, 28, 1099-1105, Amato R J. J. Clin. Can. Res. 2010; 16(22): 5539-47, Kim, D W. Hum. Vaccine. 2010; 6: 784-791, Oudard, S. Cancer Immunol. Immunother. 2011; 60: 261-71, Wyatt, L S. Aids Res. Hum. Retroviruses. 2004; 20: 645-53, Gomez, C E. Virus Research 2004; 105: 11-22, Webster, D P. Proc. Natl. Acad. Sci. 2005; 102: 4836-4, Huang, X. Vaccine 2007; 25: 8874-84, Gomez, C E. Vaccine 2007a; 25: 2863-85, Esteban M. Hum. Vaccine 2009; 5: 867-871, Gomez, C E. Curr. Gene therapy 2008; 8(2): 97-120, Whelan, K T. Plos one 2009; 4(6): 5934, Scriba, T J. Eur. Jour. Immuno. 2010; 40(1): 279-90, Corbett, M. Proc. Natl. Acad. Sci. 2008; 105: 2046-2051, Midgley, C M. J. Gen. Virol. 2008; 89: 2992-97, Von Krempelhuber, A. Vaccine 2010; 28: 1209-16, Perreau, M. J. Of Virol. 2011; Octtober: 9854-62, Pantaleo, G. Curr Opin HIV-AIDS. 2010; 5: 391-396,
   each of which is incorporated herein by reference.

In another embodiment the vaccinia virus is used in the neoplasia vaccine or immunogenic composition to express a neoantigen. (Rolph et al., Recombinant viruses as vaccines and immunological tools. Curr Opin Immunol 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In another embodiment ALVAC is used as a vector in a neoplasia vaccine or immunogenic composition. ALVAC is a canarypox virus that can be modified to express foreign transgenes and has been used as a method for vaccination against both prokaryotic and eukaryotic antigens (Honig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol Immunother 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin Cancer Res 2000; 6:2219-28; Musey L, Ding Y, Elizaga M, et al. HIV-1 vaccination administered intramuscularly can induce both systemic and mucosal T cell immunity in HIV-1-uninfected individuals. J Immunol 2003; 171:1094-101; Paoletti E. Applications of pox virus vectors to vaccination: an update. Proc Natl Acad Sci USA 1996; 93:11349-53; U.S. Pat. No. 7,255,862). In a phase I clinical trial, an ALVAC virus expressing the tumor antigen CEA showed an excellent safety profile and resulted in increased CEA-specific T-cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J Clin Oncol 1999; 17:332-7).

In another embodiment a Modified Vaccinia Ankara (MVA) virus may be used as a viral vector for a neoantigen vaccine or immunogenic composition. MVA is a member of the Orthopoxvirus family and has been generated by about 570 serial passages on chicken embryo fibroblasts of the Ankara strain of Vaccinia virus (CVA) (for review see Mayr, A., et al., Infection 3, 6-14, 1975). As a consequence of these passages, the resulting MVA virus contains 31 kilobases less genomic information compared to CVA, and is highly host-cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol Immunother. Jan 2012; 61(1): 19-29). Methods to make and use recombinant MVA has been described (e.g., see U.S. Pat. Nos. 8,309,098 and 5,185,146 hereby incorporated in its entirety).

In another embodiment the modified Copenhagen strain of vaccinia virus, NYVAC and NYVAC variations are used as a vector (see U.S. Pat. No. 7,255,862; PCT WO 95/30018; U.S. Pat. Nos. 5,364,773 and 5,494,807, hereby incorporated by reference in its entirety).

In one embodiment recombinant viral particles of the vaccine or immunogenic composition are administered to patients in need thereof. Dosages of expressed neoantigen can range from a few to a few hundred micrograms, e.g., 5 to 500 .mu.g. The vaccine or immunogenic composition can be administered in any suitable amount to achieve expression at these dosage levels. The viral particles can be administered to a patient in need thereof or transfected into cells in an amount of about at least 10³⁵ pfu; thus, the viral particles are preferably administered to a patient in need thereof or infected or transfected into cells in at least about 10⁴ pfu to about 10⁶ pfu; however, a patient in need thereof can be administered at least about 10⁸ pfu such that a more preferred amount for administration can be at least about 10⁷ pfu to about 10⁹ pfu. Doses as to NYVAC are applicable as to ALVAC, MVA, MVA-BN, and avipoxes, such as canarypox and fowlpox.

Vaccine or Immunogenic Composition Adjuvant

Effective vaccine or immunogenic compositions advantageously include a strong adjuvant to initiate an immune response. As described herein, poly-ICLC, an agonist of TLR3 and the RNA helicase—domains of MDAS and RIGS, has shown several desirable properties for a vaccine or immunogenic composition adjuvant. These properties include the induction of local and systemic activation of immune cells in vivo, production of stimulatory chemokines and cytokines, and stimulation of antigen-presentation by DCs. Furthermore, poly-ICLC can induce durable CD4+ and CD8+ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NY-ESO-1 peptide vaccine (in addition to Montanide) showed induction of CD4+ and CD8+ T cell, as well as antibody responses to the peptide in a recent phase 1 study. At the same time, poly-ICLC has been extensively tested in more than 25 clinical trials to date and exhibited a relatively benign toxicity profile. In addition to a powerful and specific immunogen the neoantigen peptides may be combined with an adjuvant (e.g., poly-ICLC) or another anti-neoplastic agent. Without being bound by theory, these neoantigens are expected to bypass central thymic tolerance (thus allowing stronger anti-tumor T cell response), while reducing the potential for autoimmunity (e.g., by avoiding targeting of normal self-antigens). An effective immune response advantageously includes a strong adjuvant to activate the immune system (Speiser and Romero, Molecularly defined vaccines for cancer immunotherapy, and protective T cell immunity Seminars in Immunol 22:144 (2010)). For example, Toll-like receptors (TLRs) have emerged as powerful sensors of microbial and viral pathogen “danger signals”, effectively inducing the innate immune system, and in turn, the adaptive immune system (Bhardwaj and Gnjatic, TLR AGONISTS: Are They Good Adjuvants? Cancer J. 16:382-391 (2010)). Among the TLR agonists, poly-ICLC (a synthetic double-stranded RNA mimic) is one of the most potent activators of myeloid-derived dendritic cells. In a human volunteer study, poly-ICLC has been shown to be safe and to induce a gene expression profile in peripheral blood cells comparable to that induced by one of the most potent live attenuated viral vaccines, the yellow fever vaccine YF-17D (Caskey et al, Synthetic double-stranded RNA induces innate immune responses similar to a live viral vaccine in humans J Exp Med 208:2357 (2011)). In a preferred embodiment Hiltonol®, a GMP preparation of poly-ICLC prepared by Oncovir, Inc, is utilized as the adjuvant. In other embodiments, other adjuvants described herein are envisioned. For instance oil-in-water, water-in-oil or multiphasic W/O/W; see, e.g., U.S. Pat. No. 7,608,279 and Aucouturier et al, Vaccine 19 (2001), 2666-2672, and documents cited therein.

### Indications

Examples of cancers and cancer conditions that can be treated with the therapy of this document include, but are not limited to a patient in need thereof that has been diagnosed as having cancer, or at risk of developing cancer. The subject may have a solid tumor such as breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas, tumors of the brain and central nervous system (e.g., tumors of the meninges, brain, spinal cord, cranial nerves and other parts of the CNS, such as glioblastomas or medulla blastomas); head and/or neck cancer, breast tumors, tumors of the circulatory system (e.g., heart, mediastinum and pleura, and other intrathoracic organs, vascular tumors, and tumor-associated vascular tissue); tumors of the blood and lymphatic system (e.g., Hodgkin's disease, Non-Hodgkin's disease lymphoma, Burkitt's lymphoma, AIDS-related lymphomas, malignant immunoproliferative diseases, multiple myeloma, and malignant plasma cell neoplasms, lymphoid leukemia, myeloid leukemia, acute or chronic lymphocytic leukemia, monocytic leukemia, other leukemias of specific cell type, leukemia of unspecified cell type, unspecified malignant neoplasms of lymphoid, hematopoietic and related tissues, such as diffuse large cell lymphoma, T-cell lymphoma or cutaneous T-cell lymphoma); tumors of the excretory system (e.g., kidney, renal pelvis, ureter, bladder, and other urinary organs); tumors of the gastrointestinal tract (e.g., esophagus, stomach, small intestine, colon, colorectal, rectosigmoid junction, rectum, anus, and anal canal); tumors involving the liver and intrahepatic bile ducts, gall bladder, and other parts of the biliary tract, pancreas, and other digestive organs; tumors of the oral cavity (e.g., lip, tongue, gum, floor of mouth, palate, parotid gland, salivary glands, tonsil, oropharynx, nasopharynx, puriform sinus, hypopharynx, and other sites of the oral cavity); tumors of the reproductive system (e.g., vulva, vagina, Cervix uteri, uterus, ovary, and other sites associated with female genital organs, placenta, penis, prostate, testis, and other sites associated with male genital organs); tumors of the respiratory tract (e.g., nasal cavity, middle ear, accessory sinuses, larynx, trachea, bronchus and lung, such as small cell lung cancer and non-small cell lung cancer); tumors of the skeletal system (e.g., bone and articular cartilage of limbs, bone articular cartilage and other sites); tumors of the skin (e.g., malignant melanoma of the skin, non-melanoma skin cancer, basal cell carcinoma of skin, squamous cell carcinoma of skin, mesothelioma, Kaposi's sarcoma); and tumors involving other tissues including peripheral nerves and autonomic nervous system, connective and soft tissue, retroperitoneoum and peritoneum, eye, thyroid, adrenal gland, and other endocrine glands and related structures, secondary and unspecified malignant neoplasms of lymph nodes, secondary malignant neoplasm of respiratory and digestive systems and secondary malignant neoplasm of other sites. Thus the population of subjects described herein may be suffering from one of the above cancer types. In other embodiments, the population of subjects may be all subjects suffering from solid tumors, or all subjects suffering from liquid tumors.

Of special interest is the treatment of Non-Hodgkin's Lymphoma (NHL), clear cell Renal Cell Carcinoma (ccRCC), metastatic melanoma, sarcoma, leukemia or a cancer of the bladder, colon, brain, breast, head and neck, endometrium, lung, ovary, pancreas or prostate. In certain embodiments, the melanoma is high risk melanoma.

Cancers that can be treated using the therapy described herein may include among others cases which are refractory to treatment with other chemotherapeutics. The term “refractory, as used herein refers to a cancer (and/or metastases thereof), which shows no or only weak antiproliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The therapy described herein is also applicable to the treatment of patients in need thereof who have not been previously treated.

The therapy described herein is also applicable where the subject has no detectable neoplasia but is at high risk for disease recurrence.

Also of special interest is the treatment of patients in need thereof who have undergone Autologous Hematopoietic Stem Cell Transplant (AHSCT), and in particular patients who demonstrate residual disease after undergoing AHSCT. The post-AHSCT setting is characterized by a low volume of residual disease, the infusion of immune cells to a situation of homeostatic expansion, and the absence of any standard relapse-delaying therapy. These features provide a unique opportunity to use the claimed neoplastic vaccine or immunogenic composition compositions to delay disease relapse.

Pharmaceutical Compositions/Methods of Delivery

The present invention is also directed to pharmaceutical compositions comprising an effective amount of one or more neoantigenic peptides as described herein (including a pharmaceutically acceptable salt, thereof), optionally in combination with a pharmaceutically acceptable carrier, excipient or additive.

When administered as a combination, the therapeutic agents (i.e. the neoantigenic peptides) can be formulated as separate compositions that are given at the same time or different times, or the therapeutic agents can be given as a single composition.

The compositions may be administered once daily, twice daily, once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compositions of the invention can be used to treat diseases and disease conditions that are acute, and may also be used for treatment of chronic conditions. In particular, the compositions of the invention are used in methods to treat or prevent a neoplasia. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

Surgical resection uses surgery to remove abnormal tissue in cancer, such as mediastinal, neurogenic, or germ cell tumors, or thymoma. In certain embodiments, administration of the composition is initiated following tumor resection. In other embodiments, administration of the neoplasia vaccine or immunogenic composition is initiated 1, 2, 3, 4, 5, 6, 7, 8, 9, 10, 11, 12, 13, 14, 15 or more weeks after tumor resection. Preferably, administration of the neoplasia vaccine or immunogenic composition is initiated 4, 5, 6, 7, 8, 9, 10, 11 or 12 weeks after tumor resection.

Prime/boost regimens refer to the successive administrations of a vaccine or immunogenic or immunological compositions. In certain embodiments, administration of the neoplasia vaccine or immunogenic composition is in a prime/boost dosing regimen, for example administration of the neoplasia vaccine or immunogenic composition at weeks 1, 2, 3 or 4 as a prime and administration of the neoplasia vaccine or immunogenic composition is at months 2, 3 or 4 as a boost. In another embodiment heterologous prime-boost strategies are used to ellicit a greater cytotoxic T-cell response (see Schneider et al., Induction of CD8+ T cells using heterologous prime-boost immunisation strategies, Immunological Reviews Volume 170, Issue 1, pages 29-38, August 1999). In another embodiment DNA encoding neoantigens is used to prime followed by a protein boost. In another embodiment protein is used to prime followed by boosting with a virus encoding the neoantigen. In another embodiment a virus encoding the neoantigen is used to prime and another virus is used to boost. In another embodiment protein is used to prime and DNA is used to boost. In a preferred embodiment a DNA vaccine or immunogenic composition is used to prime a T-cell response and a recombinant viral vaccine or immunogenic composition is used to boost the response. In another preferred embodiment a viral vaccine or immunogenic composition is coadministered with a protein or DNA vaccine or immunogenic composition to act as an adjuvant for the protein or DNA vaccine or immunogenic composition. The patient can then be boosted with either the viral vaccine or immunogenic composition, protein, or DNA vaccine or immunogenic composition (see Hutchings et al., Combination of protein and viral vaccines induces potent cellular and humoral immune responses and enhanced protection from murine malaria challenge. Infect Immun. 2007 Dec;75(12):5819-26. Epub 2007 Oct. 1).

The pharmaceutical compositions can be processed in accordance with conventional methods of pharmacy to produce medicinal agents for administration to patients in need thereof, including humans and other mammals.

Modifications of the neoantigenic peptides can affect the solubility, bioavailability and rate of metabolism of the peptides, thus providing control over the delivery of the active species. Solubility can be assessed by preparing the neoantigenic peptide and testing according to known methods well within the routine practitioner's skill in the art.

In certain embodiments of the pharmaceutical composition the pharmaceutically acceptable carrier comprises water. In certain embodiments, the pharmaceutically acceptable carrier further comprises dextrose. In certain embodiments, the pharmaceutically acceptable carrier further comprises dimethylsulfoxide. In certain embodiments, the pharmaceutical composition further comprises an immunomodulator or adjuvant. In certain embodiments, the immunodulator or adjuvant is selected from the group consisting of poly-ICLC, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In certain embodiments, the immunomodulator or adjuvant comprises poly-ICLC.

Xanthenone derivatives such as, for example, Vadimezan or AsA404 (also known as 5,6-dimethylaxanthenone-4-acetic acid (DMXAA)), may also be used as adjuvants according to embodiments of the invention. Alternatively, such derivatives may also be administered in parallel to the vaccine or immunogenic composition of the invention, for example via systemic or intratumoral delivery, to stimulate immunity at the tumor site. Without being bound by theory, it is believed that such xanthenone derivatives act by stimulating interferon (IFN) production via the stimulator of IFN gene ISTING) receptor (see e.g., Conlon et al. (2013) Mouse, but not Human STING, Binds and Signals in Response to the Vascular Disrupting Agent 5,6-Dimethylxanthenone-4-Acetic Acid, Journal of Immunology, 190:5216-25 and Kim et al. (2013) Anticancer Flavonoids are Mouse-Selective STING Agonists, 8:1396-1401).

The vaccine or immunological composition may also include an adjuvant compound chosen from the acrylic or methacrylic polymers and the copolymers of maleic anhydride and an alkenyl derivative. It is in particular a polymer of acrylic or methacrylic acid cross-linked with a polyalkenyl ether of a sugar or polyalcohol (carbomer), in particular cross-linked with an allyl sucrose or with allylpentaerythritol. It may also be a copolymer of maleic anhydride and ethylene cross-linked, for example, with divinyl ether (see U.S. Pat. No. 6,713,068 hereby incorporated by reference in its entirety).

In certain embodiments, the pH modifier can stabilize the adjuvant or immunomodulator as described herein.

In certain embodiments, a pharmaceutical composition comprises: one to five peptides, dimethylsulfoxide (DMSO), dextrose, water, succinate, poly I: poly C, poly-L-lysine, carboxymethylcellulose, and chloride. In certain embodiments, each of the one to five peptides is present at a concentration of 300 μg/ml. In certain embodiments, the pharmaceutical composition comprises ≤3% DMSO by volume. In certain embodiments, the pharmaceutical composition comprises 3.6-3.7% dextrose in water. In certain embodiments, the pharmaceutical composition comprises 3.6-3.7 mM succinate (e.g., as sodium succinate) or a salt thereof. In certain embodiments, the pharmaceutical composition comprises 0.5 mg/ml poly I: poly C. In certain embodiments, the pharmaceutical composition comprises 0.375 mg/ml poly-L-Lysine. In certain embodiments, the pharmaceutical composition comprises 1.25 mg/ml sodium carboxymethylcellulose. In certain embodiments, the pharmaceutical composition comprises 0.225% sodium chloride.

Pharmaceutical compositions comprise the herein-described tumor specific neoantigenic peptides in a therapeutically effective amount for treating diseases and conditions (e.g., a neoplasia/tumor), which have been described herein, optionally in combination with a pharmaceutically acceptable additive, carrier and/or excipient. One of ordinary skill in the art from this disclosure and the knowledge in the art will recognize that a therapeutically effective amount of one of more compounds according to the present invention may vary with the condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., ocular, oral, topical or parenteral, including gels, creams ointments, lotions and time released implantable preparations, among numerous others. In preparing pharmaceutical compositions in oral dosage form, any of the usual pharmaceutical media may be used. Thus, for liquid oral preparations such as suspensions, elixirs and solutions, suitable carriers and additives including water, glycols, oils, alcohols, flavoring agents, preservatives, coloring agents and the like may be used. For solid oral preparations such as powders, tablets, capsules, and for solid preparations such as suppositories, suitable carriers and additives including starches, sugar carriers, such as dextrose, mannitol, lactose and related carriers, diluents, granulating agents, lubricants, binders, disintegrating agents and the like may be used. If desired, the tablets or capsules may be enteric-coated or sustained release by standard techniques.

The active compound is included in the pharmaceutically acceptable carrier or diluent in an amount sufficient to deliver to a patient a therapeutically effective amount for the desired indication, without causing serious toxic effects in the patient treated.

Oral compositions generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material herein discussed, a liquid carrier such as a fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for ocular, parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose.

In certain embodiments, the pharmaceutically acceptable carrier is an aqueous solvent, i.e., a solvent comprising water, optionally with additional co-solvents. Exemplary pharmaceutically acceptable carriers include water, buffer solutions in water (such as phosphate-buffered saline (PBS), and 5% dextrose in water (D5W). In certain embodiments, the aqueous solvent further comprises dimethyl sulfoxide (DMSO), e.g., in an amount of about 1-4%, or 1-3%. In certain embodiments, the pharmaceutically acceptable carrier is isotonic (i.e., has substantially the same osmotic pressure as a body fluid such as plasma).

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid, and polylactic-co-glycolic acid (PLGA). Methods for preparation of such formulations are within the ambit of the skilled artisan in view of this disclosure and the knowledge in the art.

A skilled artisan from this disclosure and the knowledge in the art recognizes that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier usually comprises sterile water or aqueous sodium chloride solution, though other ingredients including those which aid dispersion may be included. Of course, where sterile water is to be used and maintained as sterile, the compositions and carriers are also sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

The neoplasia vaccine or immunogenic composition, and any additional agents, may be administered by injection, orally, parenterally, by inhalation spray, rectally, vaginally, or topically in dosage unit formulations containing conventional pharmaceutically acceptable carriers, adjuvants, and vehicles. The term parenteral as used herein includes, into a lymph node or nodes, subcutaneous, intravenous, intramuscular, intrasternal, infusion techniques, intraperitoneally, eye or ocular, intravitreal, intrabuccal, transdermal, intranasal, into the brain, including intracranial and intradural, into the joints, including ankles, knees, hips, shoulders, elbows, wrists, directly into tumors, and the like, and in suppository form.

In certain embodiments, the vaccine or immunogenic composition is administered intravenously or subcutaneously. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The tumor specific neoantigenic peptides may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

The tumor specific neoantigenic peptides may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prednisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; and/or RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as HERCEPTIN and RITUXAN.

It should be understood that in addition to the ingredients particularly mentioned herein, the formulations of the present invention may include other agents conventional in the art having regard to the type of formulation in question, for example, those suitable for oral administration may include flavoring agents.

Pharmaceutically acceptable salt forms may be the preferred chemical form of compounds according to the present invention for inclusion in pharmaceutical compositions according to the present invention.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

The compounds herein are commercially available or can be synthesized. As can be appreciated by the skilled artisan, further methods of synthesizing the compounds of the formulae herein is evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The additional agents that may be included with the tumor specific neo-antigenic peptides of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

### Dosage

When the agents described herein are administered as pharmaceuticals to humans or animals, they can be given per se or as a pharmaceutical composition containing active ingredient in combination with a pharmaceutically acceptable carrier, excipient, or diluent.

Actual dosage levels and time course of administration of the active ingredients in the pharmaceutical compositions of the invention can be varied so as to obtain an amount of the active ingredient which is effective to achieve the desired therapeutic response for a particular patient, composition, and mode of administration, without being toxic to the patient. Generally, agents or pharmaceutical compositions of the invention are administered in an amount sufficient to reduce or eliminate symptoms associated with neoplasia, e.g. cancer or tumors.

A preferred dose of an agent is the maximum that a patient can tolerate and not develop serious or unacceptable side effects. Exemplary dose ranges include 0.01 mg to 250 mg per day, 0.01 mg to 100 mg per day, 1 mg to 100 mg per day, 10 mg to 100 mg per day, 1 mg to 10 mg per day, and 0.01 mg to 10 mg per day. A preferred dose of an agent is the maximum that a patient can tolerate and not develop serious or unacceptable side effects. In embodiments, the agent is administered at a concentration of about 10 micrograms to about 100 mg per kilogram of body weight per day, about 0.1 to about 10 mg/kg per day, or about 1.0 mg to about 10 mg/kg of body weight per day.

In embodiments, the pharmaceutical composition comprises an agent in an amount ranging between 1 and 10 mg, such as 1, 2, 3, 4, 5, 6, 7, 8, 9, or 10 mg.

In embodiments, the therapeutically effective dosage produces a serum concentration of an agent of from about 0.1 ng/ml to about 50-100 mg/ml. The pharmaceutical compositions 5 typically should provide a dosage of from about 0.001 mg to about 2000 mg of compound per kilogram of body weight per day. For example, dosages for systemic administration to a human patient can range from 1-10 mglkg, 20-80 mglkg, 5-50 mg/kg, 75-150 mg/kg, 100-500 mg/kg, 250-750 mg/kg, 500-1000 mg/kg, 1-10 mg/kg, 5-50 mg/kg, 25-75 mg/kg, 50-100 mg/kg, 100-250 mg/kg, 50-100 mg/kg, 250-500 mg/kg, 500-750 mg/kg, 750-1000 mg/kg, 1000-1500 mg/kg, 10 1500-2000 mg/kg, 5 mg/kg, 20 mg/kg, 50 mg/kg, 100 mg/kg, 500 mg/kg, 1000 mg/kg, 1500 mg/kg, or 2000 mg/kg. Pharmaceutical dosage unit forms are prepared to provide from about 1 mg to about 5000 mg, for example from about 100 to about 2500 mg of the compound or a combination of essential ingredients per dosage unit form.

In embodiments, about 50 nM to about 1 μM of an agent is administered to a subject. In related embodiments, about 50-100 nM, 50-250 nM, 100-500 nM, 250-500 nM, 250-750 nM, 500-750 nM, 500 nM to 1 μM, or 750 nM to 1 μM of an agent is administered to a subject.

Determination of an effective amount is well within the capability of those skilled in the art, especially in light of the detailed disclosure provided herein. Generally, an efficacious or effective amount of an agent is determined by first administering a low dose of the agent(s) and then incrementally increasing the administered dose or dosages until a desired effect (e.g., reduce or eliminate symptoms associated with viral infection or autoimmune disease) is observed in the treated subject, with minimal or acceptable toxic side effects. Applicable methods for determining an appropriate dose and dosing schedule for administration of a pharmaceutical composition of the present invention are described, for example, in Goodman and Gilman's The Pharmacological Basis of Therapeutics, Goodman et al., eds., 11th Edition, McGraw-Hill 2005, and Remington: The Science and Practice of Pharmacy, 20th and 21st Editions, Gennaro and University of the Sciences in Philadelphia, Eds., Lippencott Williams & Wilkins (2003 and 2005), each of which is hereby incorporated by reference.

Preferred unit dosage formulations are those containing a daily dose or unit, daily sub-dose, as herein discussed, or an appropriate fraction thereof, of the administered ingredient.

The dosage regimen for treating a disorder or a disease with the tumor specific neoantigenic peptides of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject can depend on a number of factors, such as the mode of administration, the nature of the condition being treated, the body weight of the subject being treated and the judgment of the prescribing physician; all such factors being within the ambit of the skilled artisan from this disclosure and the knowledge in the art.

The amount of compound included within therapeutically active formulations according to the present invention is an effective amount for treating the disease or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound can depend on the condition being treated, the particular compound, and other clinical factors such as weight and condition of the patient and the route of administration of the compound. It is to be understood that the present invention has application for both human and veterinary use.

For oral administration to humans, a dosage of between approximately 0.1 to 100 mg/kg/day, preferably between approximately 1 and 100 mg/kg/day, is generally sufficient.

Where drug delivery is systemic rather than topical, this dosage range generally produces effective blood level concentrations of active compound ranging from less than about 0.04 to about 400 micrograms/cc or more of blood in the patient. The compound is conveniently administered in any suitable unit dosage form, including but not limited to one containing 0.001 to 3000 mg, preferably 0.05 to 500 mg of active ingredient per unit dosage form. An oral dosage of 10-250 mg is usually convenient.

According to certain exemplary embodiments, the vaccine or immunogenic composition is administered at a dose of about 10 μg to 1 mg per neoantigenic peptide. According to certain exemplary embodiments, the vaccine or immunogenic composition is administered at an average weekly dose level of about 10 μg to 2000 μg per neoantigenic peptide.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The invention provides for pharmaceutical compositions containing at least one tumor specific neoantigen described herein. In embodiments, the pharmaceutical compositions contain a pharmaceutically acceptable carrier, excipient, or diluent, which includes any pharmaceutical agent that does not itself induce the production of an immune response harmful to a subject receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful for treating and/or preventing viral infection and/or autoimmune disease.

A thorough discussion of pharmaceutically acceptable carriers, diluents, and other excipients is presented in Remington's Pharmaceutical Sciences (17th ed., Mack Publishing Company) and Remington: The Science and Practice of Pharmacy (21st ed., Lippincott Williams & Wilkins), which are hereby incorporated by reference. The formulation of the pharmaceutical composition should suit the mode of administration. In embodiments, the pharmaceutical composition is suitable for administration to humans, and can be sterile, non-particulate and/or non-pyrogenic.

Pharmaceutically acceptable carriers, excipients, or diluents include, but are not limited, to saline, buffered saline, dextrose, water, glycerol, ethanol, sterile isotonic aqueous buffer, and combinations thereof.

Wetting agents, emulsifiers and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically-acceptable antioxidants include, but are not limited to: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

In embodiments, the pharmaceutical composition is provided in a solid form, such as a lyophilized powder suitable for reconstitution, a liquid solution, suspension, emulsion, tablet, pill, capsule, sustained release formulation, or powder.

In embodiments, the pharmaceutical composition is supplied in liquid form, for example, in a sealed container indicating the quantity and concentration of the active ingredient in the pharmaceutical composition. In related embodiments, the liquid form of the pharmaceutical composition is supplied in a hermetically sealed container.

Methods for formulating the pharmaceutical compositions of the present invention are conventional and well known in the art (see Remington and Remington's). One of skill in the art can readily formulate a pharmaceutical composition having the desired characteristics (e.g., route of administration, biosafety, and release profile).

Methods for preparing the pharmaceutical compositions include the step of bringing into association the active ingredient with a pharmaceutically acceptable carrier and, optionally, one or more accessory ingredients. The pharmaceutical compositions can be prepared by uniformly and intimately bringing into association the active ingredient with liquid carriers, or finely divided solid carriers, or both, and then, if necessary, shaping the product. Additional methodology for preparing the pharmaceutical compositions, including the preparation of multilayer dosage forms, are described in Ansel's Pharmaceutical Dosage Forms and Drug Delivery Systems (9th ed., Lippincott Williams & Wilkins), which is hereby incorporated by reference.

Pharmaceutical compositions suitable for oral administration can be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) described herein, a derivative thereof, or a pharmaceutically acceptable salt or prodrug thereof as the active ingredient(s). The active ingredient can also be administered as a bolus, electuary, or paste.

In solid dosage forms for oral administration (e.g., capsules, tablets, pills, dragees, powders, granules and the like), the active ingredient is mixed with one or more pharmaceutically acceptable carriers, excipients, or diluents, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions can also comprise buffering agents. Solid compositions of a similar type can also be prepared using fillers in soft and hard-filled gelatin capsules, and excipients such as lactose or milk sugars, as well as high molecular weight polyethylene glycols and the like.

A tablet can be made by compression or molding, optionally with one or more accessory ingredients. Compressed tablets can be prepared using binders (for example, gelatin or hydroxypropylmethyl cellulose), lubricants, inert diluents, preservatives, disintegrants (for example, sodium starch glycolate or cross-linked sodium carboxymethyl cellulose), surface-actives, and/or dispersing agents. Molded tablets can be made by molding in a suitable machine a mixture of the powdered active ingredient moistened with an inert liquid diluent.

The tablets and other solid dosage forms, such as dragees, capsules, pills, and granules, can optionally be scored or prepared with coatings and shells, such as enteric coatings and other coatings well known in the art.

In some embodiments, in order to prolong the effect of an active ingredient, it is desirable to slow the absorption of the compound from subcutaneous or intramuscular injection. This can be accomplished by the use of a liquid suspension of crystalline or amorphous material having poor water solubility. The rate of absorption of the active ingredient then depends upon its rate of dissolution which, in turn, can depend upon crystal size and crystalline form. Alternatively, delayed absorption of a parenterally-administered active ingredient is accomplished by dissolving or suspending the compound in an oil vehicle. In addition, prolonged absorption of the injectable pharmaceutical form can be brought about by the inclusion of agents that delay absorption such as aluminum monostearate and gelatin.

Controlled release parenteral compositions can be in form of aqueous suspensions, microspheres, microcapsules, magnetic microspheres, oil solutions, oil suspensions, emulsions, or the active ingredient can be incorporated in biocompatible carrier(s), liposomes, nanoparticles, implants or infusion devices.

Materials for use in the preparation of microspheres and/or microcapsules include biodegradable/bioerodible polymers such as polyglactin, poly-(isobutyl cyanoacrylate), poly(2-hydroxyethyl-L-glutamine) and poly(lactic acid).

Biocompatible carriers which can be used when formulating a controlled release parenteral formulation include carbohydrates such as dextrans, proteins such as albumin, lipoproteins or antibodies.

Materials for use in implants can be non-biodegradable, e.g., polydimethylsiloxane, or biodegradable such as, e.g., poly(caprolactone), poly(lactic acid), poly(glycolic acid) or poly(ortho esters).

In embodiments, the active ingredient(s) are administered by aerosol. This is accomplished by preparing an aqueous aerosol, liposomal preparation, or solid particles containing the compound. A nonaqueous (e.g., fluorocarbon propellant) suspension can be used. The pharmaceutical composition can also be administered using a sonic nebulizer, which would minimize exposing the agent to shear, which can result in degradation of the compound.

Ordinarily, an aqueous aerosol is made by formulating an aqueous solution or suspension of the active ingredient(s) together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Dosage forms for topical or transdermal administration of an active ingredient(s) includes powders, sprays, ointments, pastes, creams, lotions, gels, solutions, patches and inhalants. The active ingredient(s) can be mixed under sterile conditions with a pharmaceutically acceptable carrier, and with any preservatives, buffers, or propellants as appropriate.

Transdermal patches suitable for use in the present invention are disclosed in Transdermal Drug Delivery: Developmental Issues and Research Initiatives (Marcel Dekker Inc., 1989) and U.S. Pat. Nos. 4,743,249, 4,906,169, 5,198,223, 4,816,540, 5,422,119, 5,023,084, which are hereby incorporated by reference. The transdermal patch can also be any transdermal patch well known in the art, including transscrotal patches. Pharmaceutical compositions in such transdermal patches can contain one or more absorption enhancers or skin permeation enhancers well known in the art (see, e.g., U.S. Pat. Nos. 4,379,454 and 4,973,468, which are hereby incorporated by reference). Transdermal therapeutic systems for use in the present invention can be based on iontophoresis, diffusion, or a combination of these two effects.

Transdermal patches have the added advantage of providing controlled delivery of active ingredient(s) to the body. Such dosage forms can be made by dissolving or dispersing the active ingredient(s) in a proper medium. Absorption enhancers can also be used to increase the flux of the active ingredient across the skin. The rate of such flux can be controlled by either providing a rate controlling membrane or dispersing the active ingredient(s) in a polymer matrix or gel.

Such pharmaceutical compositions can be in the form of creams, ointments, lotions, liniments, gels, hydrogels, solutions, suspensions, sticks, sprays, pastes, plasters and other kinds of transdermal drug delivery systems. The compositions can also include pharmaceutically acceptable carriers or excipients such as emulsifying agents, antioxidants, buffering agents, preservatives, humectants, penetration enhancers, chelating agents, gel-forming agents, ointment bases, perfumes, and skin protective agents.

Examples of emulsifying agents include, but are not limited to, naturally occurring gums, e.g. gum acacia or gum tragacanth, naturally occurring phosphatides, e.g. soybean lecithin and sorbitan monooleate derivatives.

Examples of antioxidants include, but are not limited to, butylated hydroxy anisole (BHA), ascorbic acid and derivatives thereof, tocopherol and derivatives thereof, and cysteine.

Examples of preservatives include, but are not limited to, parabens, such as methyl or propyl p-hydroxybenzoate and benzalkonium chloride.

Examples of humectants include, but are not limited to, glycerin, propylene glycol, sorbitol and urea.

Examples of penetration enhancers include, but are not limited to, propylene glycol, DMSO, triethanolamine, N,N-dimethylacetamide, N,N-dimethylformamide, 2-pyrrolidone and derivatives thereof, tetrahydrofurfuryl alcohol, propylene glycol, diethylene glycol monoethyl or monomethyl ether with propylene glycol monolaurate or methyl laurate, eucalyptol, lecithin, TRANSCUTOL, and AZONE.

Examples of chelating agents include, but are not limited to, sodium EDTA, citric acid and phosphoric acid.

Examples of gel forming agents include, but are not limited to, Carbopol, cellulose derivatives, bentonite, alginates, gelatin and polyvinylpyrrolidone.

In addition to the active ingredient(s), the ointments, pastes, creams, and gels of the present invention can contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain excipients such as lactose, talc, silicic acid, aluminum hydroxide, calcium silicates and polyamide powder, or mixtures of these substances. Sprays can additionally contain customary propellants, such as chlorofluorohydrocarbons, and volatile unsubstituted hydrocarbons, such as butane and propane.

Injectable depot forms are made by forming microencapsule matrices of compound(s) of the invention in biodegradable polymers such as polylactide-polyglycolide. Depending on the ratio of compound to polymer, and the nature of the particular polymer employed, the rate of compound release can be controlled. Examples of other biodegradable polymers include poly(orthoesters) and poly(anhydrides). Depot injectable formulations are also prepared by entrapping the drug in liposomes or microemulsions which are compatible with body tissue.

Subcutaneous implants are well known in the art and are suitable for use in the present invention. Subcutaneous implantation methods are preferably non-irritating and mechanically resilient. The implants can be of matrix type, of reservoir type, or hybrids thereof. In matrix type devices, the carrier material can be porous or non-porous, solid or semi-solid, and permeable or impermeable to the active compound or compounds. The carrier material can be biodegradable or may slowly erode after administration. In some instances, the matrix is non-degradable but instead relies on the diffusion of the active compound through the matrix for the carrier material to degrade. Alternative subcutaneous implant methods utilize reservoir devices where the active compound or compounds are surrounded by a rate controlling membrane, e.g., a membrane independent of component concentration (possessing zero-order kinetics). Devices consisting of a matrix surrounded by a rate controlling membrane also suitable for use.

Both reservoir and matrix type devices can contain materials such as polydimethylsiloxane, such as SILASTIC, or other silicone rubbers. Matrix materials can be insoluble polypropylene, polyethylene, polyvinyl chloride, ethylvinyl acetate, polystyrene and polymethacrylate, as well as glycerol esters of the glycerol palmitostearate, glycerol stearate, and glycerol behenate type. Materials can be hydrophobic or hydrophilic polymers and optionally contain solubilizing agents.

Subcutaneous implant devices can be slow-release capsules made with any suitable polymer, e.g., as described in U.S. Pat. Nos. 5,035,891 and 4,210,644, which are hereby incorporated by reference.

In general, at least four different approaches are applicable in order to provide rate control over the release and transdermal permeation of a drug compound. These approaches are: membrane-moderated systems, adhesive diffusion-controlled systems, matrix dispersion-type systems and microreservoir systems. It is appreciated that a controlled release percutaneous and/or topical composition can be obtained by using a suitable mixture of these approaches.

In a membrane-moderated system, the active ingredient is present in a reservoir which is totally encapsulated in a shallow compartment molded from a drug-impermeable laminate, such as a metallic plastic laminate, and a rate-controlling polymeric membrane such as a microporous or a non-porous polymeric membrane, e.g., ethylene-vinyl acetate copolymer. The active ingredient is released through the rate controlling polymeric membrane. In the drug reservoir, the active ingredient can either be dispersed in a solid polymer matrix or suspended in an unleachable, viscous liquid medium such as silicone fluid. On the external surface of the polymeric membrane, a thin layer of an adhesive polymer is applied to achieve an intimate contact of the transdermal system with the skin surface. The adhesive polymer is preferably a polymer which is hypoallergenic and compatible with the active drug substance.

In an adhesive diffusion-controlled system, a reservoir of the active ingredient is formed by directly dispersing the active ingredient in an adhesive polymer and then by, e.g., solvent casting, spreading the adhesive containing the active ingredient onto a flat sheet of substantially drug-impermeable metallic plastic backing to form a thin drug reservoir layer.

A matrix dispersion-type system is characterized in that a reservoir of the active ingredient is formed by substantially homogeneously dispersing the active ingredient in a hydrophilic or lipophilic polymer matrix. The drug-containing polymer is then molded into disc with a substantially well-defined surface area and controlled thickness. The adhesive polymer is spread along the circumference to form a strip of adhesive around the disc.

A microreservoir system can be considered as a combination of the reservoir and matrix dispersion type systems. In this case, the reservoir of the active substance is formed by first suspending the drug solids in an aqueous solution of water-soluble polymer and then dispersing the drug suspension in a lipophilic polymer to form a multiplicity of unleachable, microscopic spheres of drug reservoirs.

Any of the herein-described controlled release, extended release, and sustained release compositions can be formulated to release the active ingredient in about 30 minutes to about 1 week, in about 30 minutes to about 72 hours, in about 30 minutes to 24 hours, in about 30 minutes to 12 hours, in about 30 minutes to 6 hours, in about 30 minutes to 4 hours, and in about 3 hours to 10 hours. In embodiments, an effective concentration of the active ingredient(s) is sustained in a subject for 4 hours, 6 hours, 8 hours, 10 hours, 12 hours, 16 hours, 24 hours, 48 hours, 72 hours, or more after administration of the pharmaceutical compositions to the subject.

Vaccine or Immunogenic Compositions

The present invention is directed in some aspects to pharmaceutical compositions suitable for the prevention or treatment of cancer. In one embodiment, the composition comprises at least an immunogenic composition, e.g., a neoplasia vaccine or immunogenic composition capable of raising a specific T-cell response. The neoplasia vaccine or immunogenic composition comprises neoantigenic peptides and/or neoantigenic polypeptides corresponding to tumor specific neoantigens as described herein.

A suitable neoplasia vaccine or immunogenic composition can preferably contain a plurality of tumor specific neoantigenic peptides. In an embodiment, the vaccine or immunogenic composition can include between 1 and 100 sets of peptides, more preferably between 1 and 50 such peptides, even more preferably between 10 and 30 sets peptides, even more preferably between 15 and 25 peptides. According to another preferred embodiment, the vaccine or immunogenic composition can include at least one peptides, more preferably 2, 3, 4, or 5 peptides, In certain embodiments, the vaccine or immunogenic composition can comprise 5, 6, 7, 8, 9, 10, 11, 12, 13, 14, 15, 16, 17, 18, 19, 20, 21, 22, 23, 24, 25, 26, 27, 28, 29, or 30 different peptides.

The optimum amount of each peptide to be included in the vaccine or immunogenic composition and the optimum dosing regimen can be determined by one skilled in the art without undue experimentation. For example, the peptide or its variant may be prepared for intravenous (i.v.) injection, sub-cutaneous (s.c.) injection, intradermal (i.d.) injection, intraperitoneal (i.p.) injection, intramuscular (i.m.) injection. Preferred methods of peptide injection include s.c, i.d., i.p., i.m., and i.v. Preferred methods of DNA injection include i.d., i.m., s.c, i.p. and i.v. For example, doses of between 1 and 500 mg 50 μg and 1.5 mg, preferably 10 μg to 500 μg, of peptide or DNA may be given and can depend from the respective peptide or DNA. Doses of this range were successfully used in previous trials (Brunsvig P F, et al., Cancer Immunol Immunother. 2006; 55(12): 1553-1564; M. Staehler, et al., ASCO meeting 2007; Abstract No 3017). Other methods of administration of the vaccine or immunogenic composition are known to those skilled in the art.

In one embodiment of the present invention the different tumor specific neoantigenic peptides and/or polypeptides are selected for use in the neoplasia vaccine or immunogenic composition so as to maximize the likelihood of generating an immune attack against the neoplasias/tumors in a high proportion of subjects in the population. Without being bound by theory, it is believed that the inclusion of a diversity of tumor specific neoantigenic peptides can generate a broad scale immune attack against a neoplasia/tumor. In one embodiment, the selected tumor specific neoantigenic peptides/polypeptides are encoded by missense mutations. In a second embodiment, the selected tumor specific neoantigenic peptides/polypeptides are encoded by a combination of missense mutations and neoORF mutations. In a third embodiment, the selected tumor specific neoantigenic peptides/polypeptides are encoded by neoORF mutations.

In one embodiment in which the selected tumor specific neoantigenic peptides/polypeptides are encoded by missense mutations, the peptides and/or polypeptides are chosen based on their capability to associate with the MHC molecules of a high proportion of subjects in the population. Peptides/polypeptides derived from neoORF mutations can also be selected on the basis of their capability to associate with the MHC molecules of the patient population.

The vaccine or immunogenic composition is capable of raising a specific cytotoxic T-cells response and/or a specific helper T-cell response.

The vaccine or immunogenic composition can further comprise an adjuvant and/or a carrier. Examples of useful adjuvants and carriers are given herein herein. The peptides and/or polypeptides in the composition can be associated with a carrier such as, e.g., a protein or an antigen-presenting cell such as e.g. a dendritic cell (DC) capable of presenting the peptide to a T-cell.

Adjuvants are any substance whose admixture into the vaccine or immunogenic composition increases or otherwise modifies the immune response to the mutant peptide. Carriers are scaffold structures, for example a polypeptide or a polysaccharide, to which the neoantigenic peptides, is capable of being associated. Optionally, adjuvants are conjugated covalently or non-covalently to the peptides or polypeptides of the invention.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Thl response.

Suitable adjuvants include, but are not limited to 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL. vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines may be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

Toll like receptors (TLRs) may also be used as adjuvants, and are important members of the family of pattern recognition receptors (PRRs) which recognize conserved motifs shared by many micro-organisms, termed “pathogen-associated molecular patterns” (PAMPS). Recognition of these “danger signals” activates multiple elements of the innate and adaptive immune system. TLRs are expressed by cells of the innate and adaptive immune systems such as dendritic cells (DCs), macrophages, T and B cells, mast cells, and granulocytes and are localized in different cellular compartments, such as the plasma membrane, lysosomes, endosomes, and endolysosomes. Different TLRs recognize distinct PAMPS. For example, TLR4 is activated by LPS contained in bacterial cell walls, TLR9 is activated by unmethylated bacterial or viral CpG DNA, and TLR3 is activated by double stranded RNA. TLR ligand binding leads to the activation of one or more intracellular signaling pathways, ultimately resulting in the production of many key molecules associated with inflammation and immunity (particularly the transcription factor NF-κB and the Type-I interferons). TLR mediated DC activation leads to enhanced DC activation, phagocytosis, upregulation of activation and co-stimulation markers such as CD80, CD83, and CD86, expression of CCR7 allowing migration of DC to draining lymph nodes and facilitating antigen presentation to T cells, as well as increased secretion of cytokines such as type I interferons, IL-12, and IL-6. All of these downstream events are critical for the induction of an adaptive immune response.

Among the most promising cancer vaccine or immunogenic composition adjuvants currently in clinical development are the TLR9 agonist CpG and the synthetic double-stranded RNA (dsRNA) TLR3 ligand poly-ICLC. In preclinical studies poly-ICLC appears to be the most potent TLR adjuvant when compared to LPS and CpG due to its induction of pro-inflammatory cytokines and lack of stimulation of IL-10, as well as maintenance of high levels of co-stimulatory molecules in DCs1. Furthermore, poly-ICLC was recently directly compared to CpG in non-human primates (rhesus macaques) as adjuvant for a protein vaccine or immunogenic composition consisting of human papillomavirus (HPV)16 capsomers (Stahl-Hennig C, Eisenblatter M, Jasny E, et al. Synthetic double-stranded RNAs are adjuvants for the induction of T helper 1 and humoral immune responses to human papillomavirus in rhesus macaques. PLoS pathogens. April 2009; 5(4)).

CpG immuno stimulatory oligonucleotides have also been reported to enhance the effects of adjuvants in a vaccine or immunogenic composition setting. Without being bound by theory, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it enhances dendritic cell maturation and differentiation, resulting in enhanced activation of Thl cells and strong cytotoxic T-lymphocyte (CTL) generation, even in the absence of CD4 T-cell help. The Thl bias induced by TLR9 stimulation is maintained even in the presence of vaccine adjuvants such as alum or incomplete Freund's adjuvant (IFA) that normally promote a Th2 bias. CpG oligonucleotides show even greater adjuvant activity when formulated or co-administered with other adjuvants or in formulations such as microparticles, nano particles, lipid emulsions or similar formulations, which are especially necessary for inducing a strong response when the antigen is relatively weak. They also accelerate the immune response and enabled the antigen doses to be reduced by approximately two orders of magnitude, with comparable antibody responses to the full-dose vaccine without CpG in some experiments (Arthur M. Krieg, Nature Reviews, Drug Discovery, 5, Jun. 2006, 471-484). U.S. Pat. No. 6,406,705 B1 describes the combined use of CpG oligonucleotides, non-nucleic acid adjuvants and an antigen to induce an antigen-specific immune response. A commercially available CpG TLR9 antagonist is dSLIM (double Stem Loop Immunomodulator) by Mologen (Berlin, GERMANY), which is a preferred component of the pharmaceutical composition of the present invention. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:Cl2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives useful in the context of the present invention can readily be determined by the skilled artisan without undue experimentation. Additional adjuvants include colony-stimulating factors, such as Granulocyte Macrophage Colony Stimulating Factor (GM-CSF, sargramostim).

Poly-ICLC is a synthetically prepared double-stranded RNA consisting of polyl and polyC strands of average length of about 5000 nucleotides, which has been stabilized to thermal denaturation and hydrolysis by serum nucleases by the addition of polylysine and carboxymethylcellulose. The compound activates TLR3 and the RNA helicase-domain of MDA5, both members of the PAMP family, leading to DC and natural killer (NK) cell activation and production of a “natural mix” of type I interferons, cytokines, and chemokines. Furthermore, poly-ICLC exerts a more direct, broad host-targeted anti-infectious and possibly antitumor effect mediated by the two IFN-inducible nuclear enzyme systems, the 2′5′-OAS and the P1/eIF2a kinase, also known as the PKR (4-6), as well as RIG-I helicase and MDA5.

In rodents and non-human primates, poly-ICLC was shown to enhance T cell responses to viral antigens, cross-priming, and the induction of tumor-, virus-, and autoantigen-specific CD8+ T-cells. In a recent study in non-human primates, poly-ICLC was found to be essential for the generation of antibody responses and T-cell immunity to DC targeted or non-targeted HIV Gag p24 protein, emphasizing its effectiveness as a vaccine adjuvant.

In human subjects, transcriptional analysis of serial whole blood samples revealed similar gene expression profiles among the 8 healthy human volunteers receiving one single s.c. administration of poly-ICLC and differential expression of up to 212 genes between these 8 subjects versus 4 subjects receiving placebo. Remarkably, comparison of the poly-ICLC gene expression data to previous data from volunteers immunized with the highly effective yellow fever vaccine YF17D showed that a large number of transcriptional and signal transduction canonical pathways, including those of the innate immune system, were similarly upregulated at peak time points.

More recently, an immunologic analysis was reported on patients with ovarian, fallopian tube, and primary peritoneal cancer in second or third complete clinical remission who were treated on a phase 1 study of subcutaneous vaccination with synthetic overlapping long peptides (OLP) from the cancer testis antigen NY-ESO-1 alone or with Montanide-ISA-51, or with 1.4 mg poly-ICLC and Montanide. The generation of NY-ESO-1-specific CD4+ and CD8+ T-cell and antibody responses were markedly enhanced with the addition of poly-ICLC and Montanide compared to OLP alone or OLP and Montanide.

A vaccine or immunogenic composition according to the present invention may comprise more than one different adjuvant. Furthermore, the invention encompasses a therapeutic composition comprising any adjuvant substance including any of those herein discussed. It is also contemplated that the peptide or polypeptide, and the adjuvant can be administered separately in any appropriate sequence.

A carrier may be present independently of an adjuvant. The carrier may be covalently linked to the antigen. A carrier can also be added to the antigen by inserting DNA encoding the carrier in frame with DNA encoding the antigen. The function of a carrier can for example be to confer stability, to increase the biological activity, or to increase serum half-life. Extension of the half-life can help to reduce the number of applications and to lower doses, thus are beneficial for therapeutic but also economic reasons. Furthermore, a carrier may aid presenting peptides to T-cells. The carrier may be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier may be a physiologically acceptable carrier acceptable to humans and safe. However, tetanus toxoid and/or diptheria toxoid are suitable carriers in one embodiment of the invention. Alternatively, the carrier may be dextrans for example sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments the vaccine or immunogenic composition according to the present invention additionally contains at least one antigen presenting cell.

The antigen-presenting cell (or stimulator cell) typically has an MHC class I or II molecule on its surface, and in one embodiment is substantially incapable of itself loading the MHC class I or II molecule with the selected antigen. As is described in more detail herein, the MHC class I or II molecule may readily be loaded with the selected antigen in vitro.

CD8+ cell activity may be augmented through the use of CD4+ cells. The identification of CD4 T+ cell epitopes for tumor antigens has attracted interest because many immune based therapies against cancer may be more effective if both CD8+ and CD4+ T lymphocytes are used to target a patient's tumor. CD4+ cells are capable of enhancing CD8 T cell responses. Many studies in animal models have clearly demonstrated better results when both CD4+ and CD8+ T cells participate in anti-tumor responses (see e.g., Nishimura et al. (1999) Distinct role of antigen-specific T helper type 1 (TH1) and Th2 cells in tumor eradication in vivo. J Ex Med 190:617-27). Universal CD4+ T cell epitopes have been identified that are applicable to developing therapies against different types of cancer (see e.g., Kobayashi et al. (2008) Current Opinion in Immunology 20:221-27). For example, an HLA-DR restricted helper peptide from tetanus toxoid was used in melanoma vaccines to activate CD4+ T cells non-specifically (see e.g., Slingluff et al. (2007) Immunologic and Clinical Outcomes of a Randomized Phase II Trial of Two Multipeptide Vaccines for Melanoma in the Adjuvant Setting, Clinical Cancer Research 13(21):6386-95). It is contemplated within the scope of the invention that such CD4+ cells may be applicable at three levels that vary in their tumor specificity: 1) a broad level in which universal CD4+ epitopes (e.g., tetanus toxoid) may be used to augment CD8+ cells; 2) an intermediate level in which native, tumor-associated CD4+ epitopes may be used to augment CD8+ cells; and 3) a patient specific level in which neoantigen CD4+ epitopes may be used to augment CD8+ cells in a patient specific manner. Although current algorithms for predicting CD4 epitopes are limited in accuracy, it is a reasonable expectation that many long peptides containing predicted CD8 neoepitopes will also include CD4 epitopes. CD4 epitopes are longer than CD8 epitopes and typically are 10-12 amino acids in length although some can be longer (Kreiter et al, Mutant MHC Class II epitopes drive therapeutic immune responses to cancer, Nature (2015). Thus the neoantigenic epitopes described herein, either in the form of long peptides (>25 amino acids) or nucleic acids encoding such long peptides, may also boost CD4 responses in a tumor and patient-specific manner (level (3) above).

CD8+ cell immunity may also be generated with neoantigen loaded dendritic cell (DC) vaccine. DCs are potent antigen-presenting cells that initiate T cell immunity and can be used as cancer vaccines when loaded with one or more peptides of interest, for example, by direct peptide injection. For example, patients that were newly diagnosed with metastatic melanoma were shown to be immunized against 3 HLA-A*0201-restricted gp100 melanoma antigen-derived peptides with autologous peptide pulsed CD4OL/IFN-g-activated mature DCs via an IL-12p70-producing patient DC vaccine (see e.g., Carreno et al (2013) L-12p70-producing patient DC vaccine elicits Tc1-polarized immunity, Journal of Clinical Investigation, 123(8):3383-94 and Ali et al. (2009) In situ regulation of DC subsets and T cells mediates tumor regression in mice, Cancer Immunotherapy, 1(8):1-10). It is contemplated within the scope of the invention that neoantigen loaded DCs may be prepared using the synthetic TLR 3 agonist Polyinosinic-Polycytidylic Acid-poly-L-lysine Carboxymethylcellulose (Poly-ICLC) to stimulate the DCs. Poly-ICLC is a potent individual maturation stimulus for human DCs as assessed by an upregulation of CD83 and CD86, induction of interleukin-12 (IL-12), tumor necrosis factor (TNF), interferon gamma-induced protein 10 (IP-10), interleukin 1 (IL-1), and type I interferons (IFN), and minimal interleukin 10 (IL-10) production. DCs may be differentiated from frozen peripheral blood mononuclear cells (PBMCs) obtained by leukapheresis, while PBMCs may be isolated by Ficoll gradient centrifugation and frozen in aliquots.

Illustratively, the following 7 day activation protocol may be used. Day 1—PBMCs are thawed and plated onto tissue culture flasks to select for monocytes which adhere to the plastic surface after 1-2 hr incubation at 37° C. in the tissue culture incubator. After incubation, the lymphocytes are washed off and the adherent monocytes are cultured for 5 days in the presence of interleukin-4 (IL-4) and granulocyte macrophage-colony stimulating factor (GM-CSF) to differentiate to immature DCs. On Day 6, immature DCs are pulsed with the keyhole limpet hemocyanin (KLH) protein which serves as a control for the quality of the vaccine and may boost the immunogenicity of the vaccine. The DCs are stimulated to mature, loaded with peptide antigens, and incubated overnight. On Day 7, the cells are washed, and frozen in 1 ml aliquots containing 4-20×10(6) cells using a controlled-rate freezer. Lot release testing for the batches of DCs may be performed to meet minimum specifications before the DCs are injected into patients (see e.g., Sabado et al. (2013) Preparation of tumor antigen-loaded mature dendritic cells for immunotherapy, J. Vis Exp. Aug 1;(78). doi: 10.3791/50085).

A DC vaccine may be incorporated into a scaffold system to facilitate delivery to a patient. Therapeutic treatment of a patients neoplasia with a DC vaccine may utilize a biomaterial system that releases factors that recruit host dendritic cells into the device, differentiates the resident, immature DCs by locally presenting adjuvants (e.g., danger signals) while releasing antigen, and promotes the release of activated, antigen loaded DCs to the lymph nodes (or desired site of action) where the DCs may interact with T cells to generate a potent cytotoxic T lymphocyte response to the cancer neoantigens. Implantable biomaterials may be used to generate a potent cytotoxic T lymphocyte response against a neoplasia in a patient specific manner. The biomaterial-resident dendritic cells may then be activated by exposing them to danger signals mimicking infection, in concert with release of antigen from the biomaterial. The activated dendritic cells then migrate from the biomaterials to lymph nodes to induce a cytotoxic T effector response. This approach has previously been demonstrated to lead to regression of established melanoma in preclinical studies using a lysate prepared from tumor biopsies (see e.g., Ali et al. (2209) In situ regulation of DC subsets and T cells mediates tumor regression in mice, Cancer Immunotherapy 1(8):1-10; Ali et al. (2009) Infection-mimicking materials to program dendritic cells in situ. Nat Mater 8:151-8), and such a vaccine is currently being tested in a Phase I clinical trial recently initiated at the Dana-Farber Cancer Institute. This approach has also been shown to lead to regression of glioblastoma, as well as the induction of a potent memory response to prevent relapse, using the C6 rat glioma mode1.24 in the current proposal. The ability of such an implantable, biomatrix vaccine delivery scaffold to amplify and sustain tumor specific dendritic cell activation may lead to more robust anti-tumor immunosensitization than can be achieved by traditional subcutaneous or intra-nodal vaccine administrations.

The present invention may include any method for loading a neoantigenic peptide onto a dendritic cell. One such method applicable to the present invention is a microfluidic intracellular delivery system. Such systems cause temporary membrane disruption by rapid mechanical deformation of human and mouse immune cells, thus allowing the intracellular delivery of biomolecules (Sharei et al., 2015, PLOS ONE).

Preferably, the antigen presenting cells are dendritic cells. Suitably, the dendritic cells are autologous dendritic cells that are pulsed with the neoantigenic peptide. The peptide may be any suitable peptide that gives rise to an appropriate T-cell response. T-cell therapy using autologous dendritic cells pulsed with peptides from a tumor associated antigen is disclosed in Murphy et al. (1996) The Prostate 29, 371-380 and Tjua et al. (1997) The Prostate 32, 272-278. In certain embodiments the dendritic cells are targeted using CD141, DEC205, or XCR1 markers. CD141+XCR1+DC's were identified as a subset that may be better suited to the induction of anti-tumor responses (Bachem et al., J. Exp. Med. 207, 1273-1281 (2010); Crozat et al., J. Exp. Med. 207, 1283-1292 (2010); and Gallois & Bhardwaj, Nature Med. 16, 854-856 (2010)).

Thus, in one embodiment of the present invention the vaccine or immunogenic composition containing at least one antigen presenting cell is pulsed or loaded with one or more peptides of the present invention. Alternatively, peripheral blood mononuclear cells (PBMCs) isolated from a patient may be loaded with peptides ex vivo and injected back into the patient. As an alternative the antigen presenting cell comprises an expression construct encoding a peptide of the present invention. The polynucleotide may be any suitable polynucleotide and it is preferred that it is capable of transducing the dendritic cell, thus resulting in the presentation of a peptide and induction of immunity.

The inventive pharmaceutical composition may be compiled so that the selection, number and/or amount of peptides present in the composition covers a high proportion of subjects in the population. The selection may be dependent on the specific type of cancer, the status of the disease, earlier treatment regimens, and, of course, the HLA-haplotypes present in the patient population.

Pharmaceutical compositions comprising the peptide of the invention may be administered to an individual already suffering from cancer. In therapeutic applications, compositions are administered to a patient in an amount sufficient to elicit an effective CTL response to the tumor antigen and to cure or at least partially arrest symptoms and/or complications. An amount adequate to accomplish this is defined as “therapeutically effective dose.” Amounts effective for this use can depend on, e.g., the peptide composition, the manner of administration, the stage and severity of the disease being treated, the weight and general state of health of the patient, and the judgment of the prescribing physician, but generally range for the initial immunization (that is for therapeutic or prophylactic administration) from about 1.0 μg to about 50,000 μg of peptide for a 70 kg patient, followed by boosting dosages or from about 1.0 μg to about 10,000 μg of peptide pursuant to a boosting regimen over weeks to months depending upon the patient's response and condition and possibly by measuring specific CTL activity in the patient's blood. It should be kept in mind that the peptide and compositions of the present invention may generally be employed in serious disease states, that is, life-threatening or potentially life threatening situations, especially when the cancer has metastasized. For therapeutic use, administration should begin as soon as possible after the detection or surgical removal of tumors. This is followed by boosting doses until at least symptoms are substantially abated and for a period thereafter.

The pharmaceutical compositions (e.g., vaccine compositions) for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The invention provides compositions for parenteral administration which comprise a solution of the peptides and vaccine or immunogenic compositions are dissolved or suspended in an acceptable carrier, preferably an aqueous carrier. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

A liposome suspension containing a peptide may be administered intravenously, locally, topically, etc. in a dose which varies according to, inter alia, the manner of administration, the peptide being delivered, and the stage of the disease being treated. For targeting to the immune cells, a ligand, such as, e.g., antibodies or fragments thereof specific for cell surface determinants of the desired immune system cells, can be incorporated into the liposome.

For solid compositions, conventional or nanoparticle nontoxic solid carriers may be used which include, for example, pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10-95% of active ingredient, that is, one or more peptides of the invention, and more preferably at a concentration of 25%-75%.

For aerosol administration, the immunogenic peptides are preferably supplied in finely divided form along with a surfactant and propellant. Typical percentages of peptides are 0.01%-20% by weight, preferably 1%-10%. The surfactant can, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1%-20% by weight of the composition, preferably 0.25-5%. The balance of the composition is ordinarily propellant. A carrier can also be included as desired, as with, e.g., lecithin for intranasal delivery.

The peptides and polypeptides of the invention can be readily synthesized chemically utilizing reagents that are free of contaminating bacterial or animal substances (Merrifield RB: Solid phase peptide synthesis. I. The synthesis of a tetrapeptide. J. Am. Chem. Soc. 85:2149-54, 1963).

The peptides and polypeptides of the invention can also be expressed by a vector, e.g., a nucleic acid molecule as herein-discussed, e.g., RNA or a DNA plasmid, a viral vector such as a poxvirus, e.g., orthopox virus, avipox virus, or adenovirus, AAV or lentivirus. This approach involves the use of a vector to express nucleotide sequences that encode the peptide of the invention. Upon introduction into an acutely or chronically infected host or into a noninfected host, the vector expresses the immunogenic peptide, and thereby elicits a host CTL response.

For therapeutic or immunization purposes, nucleic acids encoding the peptide of the invention and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Generally, a plasmid for a vaccine or immunological composition can comprise DNA encoding an antigen (e.g., one or more neoantigens) operatively linked to regulatory sequences which control expression or expression and secretion of the antigen from a host cell, e.g., a mammalian cell; for instance, from upstream to downstream, DNA for a promoter, such as a mammalian virus promoter (e.g., a CMV promoter such as an hCMV or mCMV promoter, e.g., an early-intermediate promoter, or an SV40 promoter—see documents cited or incorporated herein for useful promoters), DNA for a eukaryotic leader peptide for secretion (e.g., tissue plasminogen activator), DNA for the neoantigen(s), and DNA encoding a terminator (e.g., the 3′ UTR transcriptional terminator from the gene encoding Bovine Growth Hormone or bGH polyA). A composition can contain more than one plasmid or vector, whereby each vector contains and expresses a different neoantigen. Mention is also made of Wasmoen U.S. Pat. No. 5,849,303, and Dale U.S. Pat. No. 5,811,104, whose text may be useful. DNA or DNA plasmid formulations can be formulated with or inside cationic lipids; and, as to cationic lipids, as well as adjuvants, mention is also made of Loosmore U.S. Patent Application 2003/0104008. Also, teachings in Audonnet U.S. Pat. Nos. 6,228,846 and 6,159,477 may be relied upon for DNA plasmid teachings that can be employed in constructing and using DNA plasmids that contain and express in vivo.

The nucleic acids can also be delivered complexed to cationic compounds, such as cationic lipids. Lipid-mediated gene delivery methods are described, for instance, in WO1996/18372; WO 1993/24640; Mannino & Gould-Fogerite , BioTechniques 6(7): 682-691 (1988); U.S. Pat. No. 5,279,833; WO 1991/06309; and Feigner et al., Proc. Natl. Acad. Sci. USA 84: 7413-7414 (1987).

RNA encoding the peptide of interest (e.g., mRNA) can also be used for delivery (see, e.g., Kiken et al, 2011; Su et al , 2011; see also U.S. Pat. No. 8,278,036; Halabi et al. J Clin Oncol (2003) 21:1232-1237; Petsch et al, Nature Biotechnology 2012 Dec. 7; 30(12):1210-6).

Viral vectors as described herein can also be used to deliver the neoantigenic peptides of the invention. Vectors can be administered so as to have in vivo expression and response akin to doses and/or responses elicited by antigen administration.

A preferred means of administering nucleic acids encoding the peptide of the invention uses minigene constructs encoding multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes may be improved by including synthetic (e.g. poly-alanine) or naturally-occurring flanking sequences adjacent to the CTL epitopes.

The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Standard regulatory sequences well known to those of skill in the art are included in the vector to ensure expression in the target cells. Several vector elements are required: a promoter with a down-stream cloning site for minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, U.S. Pat. Nos. 5,580,859 and 5,589,466 for other suitable promoter sequences.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences can also be considered for increasing minigene expression. It has recently been proposed that immuno stimulatory sequences (ISSs or CpGs) play a role in the immunogenicity of DNA' vaccines. These sequences could be included in the vector, outside the minigene coding sequence, if found to enhance immunogenicity.

In some embodiments, a bicistronic expression vector, to allow production of the minigene-encoded epitopes and a second protein included to enhance or decrease immunogenicity can be used. Examples of proteins or polypeptides that could beneficially enhance the immune response if co-expressed include cytokines (e.g., IL2, IL12, GM-CSF), cytokine-inducing molecules (e.g. LeIF) or costimulatory molecules. Helper (HTL) epitopes could be joined to intracellular targeting signals and expressed separately from the CTL epitopes. This would allow direction of the HTL epitopes to a cell compartment different than the CTL epitopes. If required, this could facilitate more efficient entry of HTL epitopes into the MHC class II pathway, thereby improving CTL induction. In contrast to CTL induction, specifically decreasing the immune response by co-expression of immunosuppressive molecules (e.g. TGF-β) may be beneficial in certain diseases.

Once an expression vector is selected, the minigene is cloned into the polylinker region downstream of the promoter. This plasmid is transformed into an appropriate E. coli strain, and DNA is prepared using standard techniques. The orientation and DNA sequence of the minigene, as well as all other elements included in the vector, are confirmed using restriction mapping and DNA sequence analysis. Bacterial cells harboring the correct plasmid can be stored as a master cell bank and a working cell bank.

Purified plasmid DNA can be prepared for injection using a variety of formulations. The simplest of these is reconstitution of lyophilized DNA in sterile phosphate-buffer saline (PBS). A variety of methods have been described, and new techniques may become available. As noted herein, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Target cell sensitization can be used as a functional assay for expression and MHC class I presentation of minigene-encoded CTL epitopes. The plasmid DNA is introduced into a mammalian cell line that is suitable as a target for standard CTL chromium release assays. The transfection method used is dependent on the final formulation. Electroporation can be used for “naked” DNA, whereas cationic lipids allow direct in vitro transfection. A plasmid expressing green fluorescent protein (GFP) can be co-transfected to allow enrichment of transfected cells using fluorescence activated cell sorting (FACS). These cells are then chromium-51 labeled and used as target cells for epitope-specific CTL lines. Cytolysis, detected by 51 Cr release, indicates production of MHC presentation of mini gene-encoded CTL epitopes.

In vivo immunogenicity is a second approach for functional testing of minigene DNA formulations. Transgenic mice expressing appropriate human MHC molecules are immunized with the DNA product. The dose and route of administration are formulation dependent (e.g. IM for DNA in PBS, IP for lipid-complexed DNA). Twenty-one days after immunization, splenocytes are harvested and restimulated for 1 week in the presence of peptides encoding each epitope being tested. These effector cells (CTLs) are assayed for cytolysis of peptide-loaded, chromium-51 labeled target cells using standard techniques. Lysis of target cells sensitized by MHC loading of peptides corresponding to minigene-encoded epitopes demonstrates DNA vaccine function for in vivo induction of CTLs.

Peptides may be used to elicit CTL ex vivo, as well. The resulting CTL, can be used to treat chronic tumors in patients in need thereof that do not respond to other conventional forms of therapy, or does not respond to a peptide vaccine approach of therapy. Ex vivo CTL responses to a particular tumor antigen are induced by incubating in tissue culture the patient's CTL precursor cells (CTLp) together with a source of antigen-presenting cells (APC) and the appropriate peptide. After an appropriate incubation time (typically 1-4 weeks), in which the CTLp are activated and mature and expand into effector CTL, the cells are infused back into the patient, where they destroy their specific target cell (i.e., a tumor cell). In order to optimize the in vitro conditions for the generation of specific cytotoxic T cells, the culture of stimulator cells are maintained in an appropriate serum-free medium.

Prior to incubation of the stimulator cells with the cells to be activated, e.g., precursor CD8+ cells, an amount of antigenic peptide is added to the stimulator cell culture, of sufficient quantity to become loaded onto the human Class I molecules to be expressed on the surface of the stimulator cells. In the present invention, a sufficient amount of peptide is an amount that allows about 200, and preferably 200 or more, human Class I MHC molecules loaded with peptide to be expressed on the surface of each stimulator cell. Preferably, the stimulator cells are incubated with >2μg/ml peptide. For example, the stimulator cells are incubates with >3, 4, 5, 10, 15, or more μg/ml peptide.

Resting or precursor CD8+ cells are then incubated in culture with the appropriate stimulator cells for a time period sufficient to activate the CD8+ cells. Preferably, the CD8+ cells are activated in an antigen-specific manner. The ratio of resting or precursor CD8+ (effector) cells to stimulator cells may vary from individual to individual and may further depend upon variables such as the amenability of an individual's lymphocytes to culturing conditions and the nature and severity of the disease condition or other condition for which the within-described treatment modality is used. Preferably, however, the lymphocyte: stimulator cell ratio is in the range of about 30:1 to 300:1. The effector/stimulator culture may be maintained for as long a time as is necessary to stimulate a therapeutically useable or effective number of CD8+ cells.

The induction of CTL in vitro requires the specific recognition of peptides that are bound to allele specific MHC class I molecules on APC. The number of specific MHC/peptide complexes per APC is crucial for the stimulation of CTL, particularly in primary immune responses. While small amounts of peptide/MHC complexes per cell are sufficient to render a cell susceptible to lysis by CTL, or to stimulate a secondary CTL response, the successful activation of a CTL precursor (pCTL) during primary response requires a significantly higher number of MHC/peptide complexes. Peptide loading of empty major histocompatability complex molecules on cells allows the induction of primary cytotoxic T lymphocyte responses.

Since mutant cell lines do not exist for every human MHC allele, it is advantageous to use a technique to remove endogenous MHC-associated peptides from the surface of APC, followed by loading the resulting empty MHC molecules with the immunogenic peptides of interest. The use of non-transformed (non-tumorigenic), noninfected cells, and preferably, autologous cells of patients as APC is desirable for the design of CTL induction protocols directed towards development of ex vivo CTL therapies. This application discloses methods for stripping the endogenous MHC-associated peptides from the surface of APC followed by the loading of desired peptides.

A stable MHC class I molecule is a trimeric complex formed of the following elements: 1) a peptide usually of 8-10 residues, 2) a transmembrane heavy polymorphic protein chain which bears the peptide-binding site in its al and a2 domains, and 3) a non-covalently associated non-polymorphic light chain, p2microglobuiin. Removing the bound peptides and/or dissociating the p2microglobulin from the complex renders the MHC class I molecules nonfunctional and unstable, resulting in rapid degradation. All MHC class I molecules isolated from PBMCs have endogenous peptides bound to them. Therefore, the first step is to remove all endogenous peptides bound to MHC class I molecules on the APC without causing their degradation before exogenous peptides can be added to them.

Two possible ways to free up MHC class I molecules of bound peptides include lowering the culture temperature from 37° C. to 26° C. overnight to destablize p2microglobulin and stripping the endogenous peptides from the cell using a mild acid treatment. The methods release previously bound peptides into the extracellular environment allowing new exogenous peptides to bind to the empty class I molecules. The cold-temperature incubation method enables exogenous peptides to bind efficiently to the MHC complex, but requires an overnight incubation at 26° C. which may slow the cell's metabolic rate. It is also likely that cells not actively synthesizing MHC molecules (e.g., resting PBMC) would not produce high amounts of empty surface MHC molecules by the cold temperature procedure.

Harsh acid stripping involves extraction of the peptides with trifluoroacetic acid, pH 2, or acid denaturation of the immunoaffinity purified class I-peptide complexes. These methods are not feasible for CTL induction, since it is important to remove the endogenous peptides while preserving APC viability and an optimal metabolic state which is critical for antigen presentation. Mild acid solutions of pH 3 such as glycine or citrate-phosphate buffers have been used to identify endogenous peptides and to identify tumor associated T cell epitopes. The treatment is especially effective, in that only the MHC class I molecules are destabilized (and associated peptides released), while other surface antigens remain intact, including MHC class II molecules. Most importantly, treatment of cells with the mild acid solutions do not affect the cell's viability or metabolic state. The mild acid treatment is rapid since the stripping of the endogenous peptides occurs in two minutes at 4° C. and the APC is ready to perform its function after the appropriate peptides are loaded. The technique is utilized herein to make peptide-specific APCs for the generation of primary antigen-specific CTL. The resulting APC are efficient in inducing peptide-specific CD8+ CTL.

Activated CD8+ cells may be effectively separated from the stimulator cells using one of a variety of known methods. For example, monoclonal antibodies specific for the stimulator cells, for the peptides loaded onto the stimulator cells, or for the CD8+ cells (or a segment thereof) may be utilized to bind their appropriate complementary ligand. Antibody-tagged molecules may then be extracted from the stimulator-effector cell admixture via appropriate means, e.g., via well-known immunoprecipitation or immunoassay methods.

Effective, cytotoxic amounts of the activated CD8+ cells can vary between in vitro and in vivo uses, as well as with the amount and type of cells that are the ultimate target of these killer cells. The amount can also vary depending on the condition of the patient and should be determined via consideration of all appropriate factors by the practitioner. Preferably, however, about 1×10⁶ to about 1×10¹², more preferably about 1×10⁸ to about 1×10¹¹, and even more preferably, about 1×10⁹ to about 1×10¹⁰ activated CD8+ cells are utilized for adult humans, compared to about 5×10⁶-5×10⁷ cells used in mice.

Preferably, as discussed herein, the activated CD8+ cells are harvested from the cell culture prior to administration of the CD8+ cells to the individual being treated. It is important to note, however, that unlike other present and proposed treatment modalities, the present method uses a cell culture system that is not tumorigenic. Therefore, if complete separation of stimulator cells and activated CD8+ cells are not achieved, there is no inherent danger known to be associated with the administration of a small number of stimulator cells, whereas administration of mammalian tumor-promoting cells may be extremely hazardous.

Methods of re-introducing cellular components are known in the art and include procedures such as those exemplified in U.S. Pat. No. 4,844,893 to Honsik, et al. and U.S. Pat. No. 4,690,915 to Rosenberg. For example, administration of activated CD8+ cells via intravenous infusion is appropriate.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments are discussed in the sections that follow.

Therapeutic Methods

The present invention provides methods of inducing a neoplasia/tumor specific immune response in a subject, vaccinating against a neoplasia/tumor, treating and or alleviating a symptom of cancer in a subject by administering the subject a plurality of neoantigenic peptides or composition of the invention.

According to the invention, the herein-described neoplasia vaccine or immunogenic composition may be used for a patient that has been diagnosed as having cancer, or at risk of developing cancer.

The claimed combination of the invention is administered in an amount sufficient to induce a CTL response.

Additional Therapies

The tumor specific neoantigen peptides and pharmaceutical compositions described herein can also be administered in a combination therapy with another agent, for example a therapeutic agent. In certain embodiments, the additional agents can be, but are not limited to, chemotherapeutic agents, anti-angiogenesis agents and agents that reduce immune-suppression.

The neoplasia vaccine or immunogenic composition can be administered before, during, or after administration of the additional agent. In embodiments, the neoplasia vaccine or immunogenic composition is administered before the first administration of the additional agent. In other embodiments, the neoplasia vaccine or immunogenic composition is administered after the first administration of the additional therapeutic agent (e.g., 1, 2, 3, 4, 5, 6, 7, 8, 9, 10, 11, 12, 13, 14 days or more). In embodiments, the neoplasia vaccine or immunogenic composition is administered simultaneously with the first administration of the additional therapeutic agent.

The therapeutic agent is for example, a chemotherapeutic or biotherapeutic agent, radiation, or immunotherapy. Any suitable therapeutic treatment for a particular cancer may be administered. Examples of chemotherapeutic and biotherapeutic agents include, but are not limited to, an angiogenesis inhibitor, such ashydroxy angiostatin K1-3, DL-α-Difluoromethyl-ornithine, endostatin, fumagillin, genistein, minocycline, staurosporine, and thalidomide; a DNA intercaltor/cross-linker, such as Bleomycin, Carboplatin, Carmustine, Chlorambucil, Cyclophosphamide, cis-Diammineplatinum(II) dichloride (Cisplatin), Melphalan, Mitoxantrone, and Oxaliplatin; a DNA synthesis inhibitor, such as (±)-Amethopterin (Methotrexate), 3-Amino-1,2,4-benzotriazine 1,4-dioxide, Aminopterin, Cytosine β-D-arabinofuranoside, 5-Fluoro-5′-deoxyuridine, 5-Fluorouracil, Ganciclovir, Hydroxyurea, and Mitomycin C; a DNA-RNA transcription regulator, such as Actinomycin D, Daunorubicin, Doxorubicin, Homoharringtonine, and Idarubicin; an enzyme inhibitor, such as S(+)-Camptothecin, Curcumin, (−)-Deguelin, 5,6-Dichlorobenzimidazole 1-β-D-ribofuranoside, Etoposide, Formestane, Fostriecin, Hispidin, 2-Imino-1-imidazoli-dineacetic acid (Cyclocreatine), Mevinolin, Trichostatin A, Tyrphostin AG 34, and Tyrphostin AG 879; a gene regulator, such as 5-Aza-2′-deoxycytidine, 5-Azacytidine, Cholecalciferol (Vitamin D3), 4-Hydroxytamoxifen, Melatonin, Mifepristone, Raloxifene, all trans-Retinal (Vitamin A aldehyde), Retinoic acid all trans (Vitamin A acid), 9-cis-Retinoic Acid, 13-cis-Retinoic acid, Retinol (Vitamin A), Tamoxifen, and Troglitazone; a microtubule inhibitor, such as Colchicine, docetaxel, Dolastatin 15, Nocodazole, Paclitaxel, Podophyllotoxin, Rhizoxin, Vinblastine, Vincristine, Vindesine, and Vinorelbine (Navelbine); and an unclassified therapeutic agent, such as 17-(Allylamino)-17-demethoxygeldanamycin, 4-Amino-1,8-naphthalimide, Apigenin, Brefeldin A, Cimetidine, Dichloromethylene-diphosphonic acid, Leuprolide (Leuprorelin), Luteinizing Hormone-Releasing Hormone, Pifithrin-a, Rapamycin, Sex hormone-binding globulin, Thapsigargin, and Urinary trypsin inhibitor fragment (Bikunin). The therapeutic agent may be altretamine, amifostine, asparaginase, capecitabine, cladribine, cisapride, cytarabine, dacarbazine (DTIC), dactinomycin, dronabinol, epoetin alpha, filgrastim, fludarabine, gemcitabine, granisetron, ifosfamide, irinotecan, lansoprazole, levamisole, leucovorin, megestrol, mesna, metoclopramide, mitotane, omeprazole, ondansetron, pilocarpine, prochloroperazine, or topotecan hydrochloride. The therapeutic agent may be a monoclonal antibody or small molecule such as rituximab (Rituxan®), alemtuzumab (Campath®), Bevacizumab (Avastin®), Cetuximab (Erbitux®), panitumumab (Vectibix®), and trastuzumab (Herceptin®), Vemurafenib (Zelboraf®) imatinib mesylate (Gleevec®), erlotinib (Tarceva®), gefitinib (Iressa®), Vismodegib (Erivedge™), 90Y-ibritumomab tiuxetan, 131I-tositumomab, ado-trastuzumab emtansine, lapatinib (Tykerb®), pertuzumab (Perjeta™), ado-trastuzumab emtansine (Kadcyla™) regorafenib (Stivarga®), sunitinib (Sutent®), Denosumab (Xgeva®), sorafenib (Nexavar®), pazopanib (Votrient®), axitinib (Inlyta®), dasatinib (Sprycel®), nilotinib (Tasigna®), bosutinib (Bosulif®), ofatumumab (Arzerra®), obinutuzumab (Gazyva™), ibrutinib (Imbruvica™) idelalisib (Zydelig®), crizotinib (Xalkori®), erlotinib (Tarceva®), afatinib dimaleate (Gilotrif®), ceritinib (LDK378/Zykadia), Tositumomab and 131I-tositumomab (Bexxar®), ibritumomab tiuxetan (Zevalin®), brentuximab vedotin (Adcetris®), bortezomib (Velcade®), siltuximab (Sylvant™), trametinib (Mekinist®), dabrafenib (Tafinlar®), pembrolizumab (Keytruda®), carfilzomib (Kyprolis®), Ramucirumab (Cyramza™), Cabozantinib (Cometriq™), vandetanib (Caprelsa®), Optionally, the therapeutic agent is a neoantigen. The therapeutic agent may be a cytokine such as interferons (INFs), interleukins (ILs), or hematopoietic growth factors. The therapeutic agent may be INF-α, IL-2, Aldesleukin, IL-2, Erythropoietin, Granulocyte-macrophage colony-stimulating factor (GM-CSF) or granulocyte colony-stimulating factor. The therapeutic agent may be a targeted therapy such as toremifene (Fareston®), fulvestrant (Faslodex®), anastrozole (Arimidex®), exemestane (Aromasin®), letrozole (Femara®), ziv-aflibercept (Zaltrap®), Alitretinoin (Panretin®), temsirolimus (Torisel®), Tretinoin (Vesanoid®), denileukin diftitox (Ontak®), vorinostat (Zolinza®), romidepsin (Istodax®), bexarotene (Targretin®), pralatrexate (Folotyn®), lenaliomide (Revlimid®), belinostat (Beleodag™) lenaliomide (Revlimid®), pomalidomide (Pomalyst®), Cabazitaxel (Jevtana®), enzalutamide (Xtandi®), abiraterone acetate (Zytiga®), radium 223 chloride (Xofigo®), or everolimus (Afinitor®). Aditionally, the therapeutic agent may be an epigenetic targeted drug such as HDAC inhibitors, kinase inhibitors, DNA methyltransferase inhibitors, histone demethylase inhibitors, or histone methylation inhibitors. The epigenetic drugs may be Azacitidine (Vidaza), Decitabine (Dacogen), Vorinostat (Zolinza), Romidepsin (Istodax), or Ruxolitinib (Jakafi). For prostate cancer treatment, a preferred chemotherapeutic agent with which anti-CTLA-4 can be combined is paclitaxel (TAXOL).

In certain embodiments, the one or more additional agents are one or more anti-glucocorticoid-induced tumor necrosis factor family receptor (GITR) agonistic antibodies. GITR is a costimulatory molecule for T lymphocytes, modulates innate and adaptive immune system and has been found to participate in a variety of immune responses and inflammatory processes. GITR was originally described by Nocentini et al. after being cloned from dexamethasone-treated murine T cell hybridomas (Nocentini et al. Proc Natl Acad Sci USA 94:6216-6221.1997). Unlike CD28 and CTLA-4, GITR has a very low basal expression on naive CD4+ and CD8+ T cells (Ronchetti et al. Eur J Immunol 34:613-622. 2004). The observation that GITR stimulation has immunostimulatory effects in vitro and induced autoimmunity in vivo prompted the investigation of the antitumor potency of triggering this pathway. A review of Modulation Of Ctla 4 And Gitr For Cancer Immunotherapy can be found in Cancer Immunology and Immunotherapy (Avogadri et al. Current Topics in Microbiology and Immunology 344. 2011). Other agents that can contribute to relief of immune suppression include checkpoint inhibitors targeted at another member of the CD28/CTLA4 Ig superfamily such as BTLA, LAGS, ICOS, PDL1 or KIR (Page et a, Annual Review of Medicine 65:27 (2014)). In further additional embodiments, the checkpoint inhibitor is targeted at a member of the TNFR superfamily such as CD40, OX40, CD137, GITR, CD27 or TIM-3. In some cases targeting a checkpoint inhibitor is accomplished with an inhibitory antibody or similar molecule. In other cases, it is accomplished with an agonist for the target; examples of this class include the stimulatory targets OX40 and GITR.

In certain embodiments, the one or more additional agents are synergistic in that they increase immunogenicity after treatment. In one embodiment the additional agent allows for lower toxicity and/or lower discomfort due to lower doses of the additional therapeutic agents or any components of the combination therapy described herein. In another embodiment the additional agent results in longer lifespan due to increased effectiveness of the combination therapy described herein. Chemotherapeutic treatments that enhance the immunological response in a patient have been reviewed (Zitvogel et al., Immunological aspects of cancer chemotherapy. Nat Rev Immunol. 2008 January; 8(1):59-73). Aditionally, chemotherapeutic agents can be administered safely with immunotherapy without inhibiting vaccine specific T-cell responses (Perez et al., A new era in anticancer peptide vaccines. Cancer May 2010). In one embodiment the additional agent is administered to increase the efficacy of the therapy described herein. In one embodiment the additional agent is a chemotherapy treatment. In one embodiment low doses of chemotherapy potentiate delayed-type hypersensitivity (DTH) responses. In one embodiment the chemotheray agent targets regulatory T-cells. In one embodiment cyclophosphamide is the therapeutic agent. In one embodiment cyclophosphamide is administered prior to vaccination. In one embodiment cyclophosphamide is administered as a single dose before vaccination (Walter et al., Multipeptide immune response to cancer vaccine IMA901 after single-dose cyclophosphamide associates with longer patient survival. Nature Medicine; 18:8 2012). In another embodiment, cyclophosphamide is administered according to a metronomic program, where a daily dose is administered for one month (Ghiringhelli et al., Metronomic cyclophosphamide regimen selectively depletes CD4+CD25+ regulatory T cells and restores T and NK effector functions in end stage cancer patients. Cancer Immunol Immunother 2007 56:641-648). In another embodiment taxanes are administered before vaccination to enhance T-cell and NK-cell functions (Zitvogel et al., 2008, Nat. Rev. Immunol., 8(1):59-73). In another embodiment a low dose of a chemotherapeutic agent is administered with the therapy described herein. In one embodiment the chemotherapeutic agent is estramustine. In one embodiment the cancer is hormone resistant prostate cancer. A >50% decrease in serum prostate specific antigen (PSA) was seen in 8.7% of advanced hormone refractory prostate cancer patients by personalized vaccination alone, whereas such a decrease was seen in 54% of patients when the personalized vaccination was combined with a low dose of estramustine (Itoh et al., Personalized peptide vaccines: A new therapeutic modality for cancer. Cancer Sci 2006; 97: 970-976). In another embodiment glucocorticoids are administered with or before the therapy described herein (Zitvogel et al., 2008, Nat. Rev. Immunol., 8(1):59-73). In another embodiment glucocorticoids are administered after the therapy described herein. In another embodiment Gemcitabine is administered before, simultaneously, or after the therapy described herein to enhance the frequency of tumor specific CTL precursors (Zitvogel et al., 2008, Nat. Rev. Immunol., 8(1):59-73). In another embodiment 5-fluorouracil is administered with the therapy described herein as synergistic effects were seen with a peptide based vaccine (Zitvogel et al., 2008, Nat. Rev. Immunol., 8(1):59-73). In another embodiment an inhibitor of Braf, such as Vemurafenib, is used as an additional agent. Braf inhibition has been shown to be associated with an increase in melanoma antigen expression and T-cell infiltrate and a decrease in immunosuppressive cytokines in tumors of treated patients (Frederick et al., BRAF inhibition is associated with enhanced melanoma antigen expression and a more favorable tumor microenvironment in patients with metastatic melanoma. Clin Cancer Res. 2013; 19:1225-1231). In another embodiment an inhibitor of tyrosine kinases is used as an additional agent. In one embodiment the tyrosine kinase inhibitor is used before vaccination with the therapy described herein. In one embodiment the tyrosine kinase inhibitor is used simultaneously with the therapy described herein. In another embodiment the tyrosine kinase inhibitor is used to create a more immune permissive environment. In another embodiment the tyrosine kinase inhibitor is sunitinib or imatinib mesylate. It has previously been shown that favorable outcomes could be achieved with sequential administration of continuous daily dosing of sunitinib and recombinant vaccine (Farsaci et al., Consequence of dose scheduling of sunitinib on host immune response elements and vaccine combination therapy. Int J Cancer; 130: 1948-1959). Sunitinib has also been shown to reverse type-1 immune suppression using a daily dose of 50 mg/day (Finke et al., Sunitinib Reverses Type-1 Immune Suppression and Decreases T-Regulatory Cells in Renal Cell Carcinoma Patients. Clin Cancer Res 2008; 14(20)). In another embodiment targeted therapies are administered in combination with the therapy described herein. Doses of targeted therapies has been described previously (Alvarez, Present and future evolution of advanced breast cancer therapy. Breast Cancer Research 2010, 12(Suppl 2):S1). In another embodiment temozolomide is administered with the therapy described herein. In one embodiment temozolomide is administered at 200 mg/day for 5 days every fourth week of a combination therapy with the therapy described herein. Results of a similar strategy have been shown to have low toxicity (Kyte et al., Telomerase Peptide Vaccination Combined with Temozolomide: A Clinical Trial in Stage IV Melanoma Patients. Clin Cancer Res; 17(13) 2011). In another embodiment the therapy is administered with an additional therapeutic agent that results in lymphopenia. In one embodiment the additional agent is temozolomide. An immune response can still be induced under these conditions (Sampson et al., Greater chemotherapy-induced lymphopenia enhances tumor-specific immune responses that eliminate EGFRvIII-expressing tumor cells in patients with glioblastoma. Neuro-Oncology 13(3):324-333, 2011).

Patients in need thereof may receive a series of priming vaccinations with a mixture of tumor-specific peptides. Additionally, over a 4 week period the priming may be followed by two boosts during a maintenance phase. All vaccinations are subcutaneously delivered. The vaccine or immunogenic composition is evaluated for safety, tolerability, immune response and clinical effect in patients and for feasibility of producing vaccine or immunogenic composition and successfully initiating vaccination within an appropriate time frame. The first cohort can consist of 5 patients, and after safety is adequately demonstrated, an additional cohort of 10 patients may be enrolled. Peripheral blood is extensively monitored for peptide-specific T-cell responses and patients are followed for up to two years to assess disease recurrence.

Administering a Combination Therapy Consistent with Standard of Care

In another aspect, the therapy described herein provides selecting the appropriate point to administer a combination therapy in relation to and within the standard of care for the cancer being treated for a patient in need thereof. The studies described herein show that the combination therapy can be effectively administered even within the standard of care that includes surgery, radiation, or chemotherapy. The standards of care for the most common cancers can be found on the website of National Cancer Institute (www.cancer.gov/cancertopics). The standard of care is the current treatment that is accepted by medical experts as a proper treatment for a certain type of disease and that is widely used by healthcare professionals. Standard or care is also called best practice, standard medical care, and standard therapy. Standards of Care for cancer generally include surgery, lymph node removal, radiation, chemotherapy, targeted therapies, antibodies targeting the tumor, and immunotherapy. Immunotherapy can include checkpoint blockers (CBP), chimeric antigen receptors (CARs), and adoptive T-cell therapy. The combination therapy described herein can be incorporated within the standard of care. The combination therapy described herein may also be administered where the standard of care has changed due to advances in medicine.

Incorporation of the combination therapy described herein may depend on a treatment step in the standard of care that can lead to activation of the immune system. Treatment steps that can activate and function synergistically with the combination therapy have been described herein. The therapy can be advantageously administered simultaneously or after a treatment that activates the immune system.

Incorporation of the combination therapy described herein may depend on a treatment step in the standard of care that causes the immune system to be suppressed. Such treatment steps may include irradiation, high doses of alkylating agents and/or methotrexate, steroids such as glucosteroids, surgery, such as to remove the lymph nodes, imatinib mesylate, high doses of TNF, and taxanes (Zitvogel et al., 2008, Nat. Rev. Immunol., 8(1):59-73). The combination therapy may be administered before such steps or may be administered after.

In one embodiment the combination therapy may be administered after bone marrow transplants and peripheral blood stem cell transplantation. Bone marrow transplantation and peripheral blood stem cell transplantation are procedures that restore stem cells that were destroyed by high doses of chemotherapy and/or radiation therapy. After being treated with high-dose anticancer drugs and/or radiation, the patient receives harvested stem cells, which travel to the bone marrow and begin to produce new blood cells. A “mini-transplant” uses lower, less toxic doses of chemotherapy and/or radiation to prepare the patient for transplant. A “tandem transplant” involves two sequential courses of high-dose chemotherapy and stem cell transplant. In autologous transplants, patients receive their own stem cells. In syngeneic transplants, patients receive stem cells from their identical twin. In allogeneic transplants, patients receive stem cells from their brother, sister, or parent. A person who is not related to the patient (an unrelated donor) also may be used. In some types of leukemia, the graft-versus-tumor (GVT) effect that occurs after allogeneic BMT and PBSCT is crucial to the effectiveness of the treatment. GVT occurs when white blood cells from the donor (the graft) identify the cancer cells that remain in the patient's body after the chemotherapy and/or radiation therapy (the tumor) as foreign and attack them. Immunotherapy with the combination therapy described herein can take advantage of this by vaccinating after a transplant. Additionally, the transferred cells may be presented with neoantigens of the combination therapy described herein before transplantation.

In one embodiment the combination therapy is administered to a patient in need thereof with a cancer that requires surgery. In one embodiment the combination therapy described herein is administered to a patient in need thereof in a cancer where the standard of care is primarily surgery followed by treatment to remove possible micro-metastases, such as breast cancer. Breast cancer is commonly treated by various combinations of surgery, radiation therapy, chemotherapy, and hormone therapy based on the stage and grade of the cancer. Adjuvant therapy for breast cancer is any treatment given after primary therapy to increase the chance of long-term survival. Neoadjuvant therapy is treatment given before primary therapy. Adjuvant therapy for breast cancer is any treatment given after primary therapy to increase the chance of long-term disease-free survival. Primary therapy is the main treatment used to reduce or eliminate the cancer. Primary therapy for breast cancer usually includes surgery, a mastectomy (removal of the breast) or a lumpectomy (surgery to remove the tumor and a small amount of normal tissue around it; a type of breast-conserving surgery). During either type of surgery, one or more nearby lymph nodes are also removed to see if cancer cells have spread to the lymphatic system. When a woman has breast-conserving surgery, primary therapy almost always includes radiation therapy. Even in early-stage breast cancer, cells may break away from the primary tumor and spread to other parts of the body (metastasize). Therefore, doctors give adjuvant therapy to kill any cancer cells that may have spread, even if they cannot be detected by imaging or laboratory tests.

In one embodiment the combination therapy is administered consistent with the standard of care for Ductal carcinoma in situ (DCIS). The standard of care for this breast cancer type is:
1. Breast-conserving surgery and radiation therapy with or without tamoxifen.
2. Total mastectomy with or without tamoxifen.
3. Breast-conserving surgery without radiation therapy.

The combination therapy may be administered before breast conserving surgery or total mastectomy to shrink the tumor before surgery. In another embodiment the combination therapy can be administered as an adjuvant therapy to remove any remaining cancer cells.

In another embodiment patients diagnosed with stage I, II, IIIA, and Operable IIIC breast cancer are treated with the combination therapy as described herein. The standard of care for this breast cancer type is:
1. Local-regional treatment:
Breast-conserving therapy (lumpectomy, breast radiation, and surgical staging of the axilla).
Modified radical mastectomy (removal of the entire breast with level I-II axillary dissection) with or without breast reconstruction.
Sentinel node biopsy.
2. Adjuvant radiation therapy postmastectomy in axillary node-positive tumors:
For one to three nodes: unclear role for regional radiation (infra/supraclavicular nodes, internal mammary nodes, axillary nodes, and chest wall).
For more than four nodes or extranodal involvement: regional radiation is advised.
3. Adjuvant systemic therapy

In one embodiment the combination therapy is administered as a neoadjuvant therapy to shrink the tumor. In another embodiment the combination is administered as an adjuvant systemic therapy.

In another embodiment patients diagnosed with inoperable stage IIIB or IIIC or inflammatory breast cancer are treated with the combination therapy as described herein. The standard of care for this breast cancer type is:
1. Multimodality therapy delivered with curative intent is the standard of care for patients with clinical stage IIIB disease.
2. Initial surgery is generally limited to biopsy to permit the determination of histology, estrogen-receptor (ER) and progesterone-receptor (PR) levels, and human epidermal growth factor receptor 2 (HER2/neu) overexpression. Initial treatment with anthracycline-based chemotherapy and/or taxane-based therapy is standard. For patients who respond to neoadjuvant chemotherapy, local therapy may consist of total mastectomy with axillary lymph node dissection followed by postoperative radiation therapy to the chest wall and regional lymphatics. Breast-conserving therapy can be considered in patients with a good partial or complete response to neoadjuvant chemotherapy. Subsequent systemic therapy may consist of further chemotherapy. Hormone therapy should be administered to patients whose tumors are ER-positive or unknown. All patients should be considered candidates for clinical trials to evaluate the most appropriate fashion in which to administer the various components of multimodality regimens.

In one embodiment the combination therapy is administered as part of the various components of multimodality regimens. In another embodiment the combination therapy is administered before, simultaneously with, or after the multimodality regimens. In another embodiment the combination therapy is administered based on synergism between the modalities. In another embodiment the combination therapy is administered after treatment with anthracycline-based chemotherapy and/or taxane-based therapy (Zitvogel et al., 2008, Nat. Rev. Immunol., 8(1):59-73). Treatment after administering the combination therapy may negatively affect dividing effector T-cells. The combination therapy may also be administered after radiation.

In another embodiment the combination therapy described herein is used in the treatment in a cancer where the standard of care is primarily not surgery and is primarily based on systemic treatments, such as Chronic Lymphocytic Leukemia (CLL).

In another embodiment patients diagnosed with stage I, II, III, and IV Chronic Lymphocytic Leukemia are treated with the combination therapy as described herein. The standard of care for this cancer type is:
1. Observation in asymptomatic or minimally affected patients
2. Rituximab
3. Ofatumomab
4. Oral alkylating agents with or without corticosteroids
5. Fludarabine, 2-chlorodeoxyadenosine, or pentostatin
6. Bendamustine
7. Lenalidomide
8. Combination chemotherapy.
   combination chemotherapy regimens include the following:
   Fludarabine plus cyclophosphamide plus rituximab.
   Fludarabine plus rituximab as seen in the CLB-9712 and CLB-9011 trials.
   Fludarabine plus cyclophosphamide versus fludarabine plus cyclophosphamide plus rituximab.
   Pentostatin plus cyclophosphamide plus rituximab as seen in the MAYO-MC0183 trial, for example.
   Ofatumumab plus fludarabine plus cyclophosphamide.
   CVP: cyclophosphamide plus vincristine plus prednisone.
   CHOP: cyclophosphamide plus doxorubicin plus vincristine plus prednisone.
   Fludarabine plus cyclophosphamide versus fludarabine as seen in the E2997 trial [NCT00003764] and the LRF-CLL4 trial, for example.
   Fludarabine plus chlorambucil as seen in the CLB-9011 trial, for example.
9. Involved-field radiation therapy.
10. Alemtuzumab
11. Bone marrow and peripheral stem cell transplantations are under clinical evaluation.
12. Ibrutinib

In one embodiment the combination therapy is administered before, simultaneously with or after treatment with Rituximab or Ofatumomab. As these are monoclonal antibodies that target B-cells, treatment with the combination therapy may be synergistic. In another embodiment the combination therapy is administered after treatment with oral alkylating agents with or without corticosteroids, and Fludarabine, 2-chlorodeoxyadenosine, or pentostatin, as these treatments may negatively affect the immune system if administered before. In one embodiment bendamustine is administered with the combination therapy in low doses based on the results for prostate cancer described herein. In one embodiment the combination therapy is administered after treatment with bendamustine.

In another embodiment, therapies targeted to specific recurrent mutations in genes that include extracellular domains are used in the treatment of a patient in need thereof suffering from cancer. The genes may advantageously be well-expressed genes. Well expressed may be expressed in “transcripts per million” (TPM). A TPM greater than 100 is considered well expressed. Well expressed genes may be FGFR3, ERBB3, EGFR, MUC4, PDGFRA, MMP12, TMEM52, and PODXL. The therapies may be a ligand capable of binding to an extracellular neoantigen epitope. Such ligands are well known in the art and may include therapeutic antibodies or fragments thereof, antibody-drug conjugates, engineered T cells, or aptamers. Engineered T cells may be chimeric antigen receptors (CARs). Antibodies may be fully humanized, humanized, or chimeric. The antibody fragments may be a nanobody, Fab, Fab′, (Fab′)2, Fv, ScFv, diabody, triabody, tetrabody, Bis-scFv, minibody, Fab2, or Fab3 fragment. Antibodies may be developed against tumor-specific neoepitopes using known methods in the art.

Adoptive Cell Transfer (ACT)

Aspects of the invention involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322). Alternative CAR constructs may be characterized as belonging to successive generations. First-generation CARs typically consist of a single-chain variable fragment of an antibody specific for an antigen, for example comprising a V_{L} linked to a V_{H} of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/0X40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, or CD28 signaling domains (for example scFv-CD28-4-1BB-CD3ζ or scFv-CD28-OX40-CD3; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3t and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-y). CAR T cells of this kind may for example be used in animal models, for example to treat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction).

In one embodiment, the treatment can be administrated into patients undergoing an immunosuppressive treatment. The cells or population of cells, may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). Cells may be edited using any DNA targeting protein, including, but not limited to a CRISPR system, Zinc Finger binding protein, TALE or TALEN as known in the art. DNA targeting proteins may be delivered to an immune cell by any method known in the art. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

By inactivating a gene it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art.

T cell receptors (TCR) are cell surface receptors that participate in the activation of T cells in response to the presentation of antigen. The TCR is generally made from two chains, a and (3, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAGS, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson HA, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1 or TIM-3. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

### Vaccine or Immunogenic Composition Kits and Co-Packaging

In an aspect, the invention provides kits containing any one or more of the elements discussed herein to allow administration of the therapy. Elements may be provided individually or in combinations, and may be provided in any suitable container, such as a vial, a bottle, or a tube. In some embodiments, the kit includes instructions in one or more languages, for example in more than one language. In some embodiments, a kit comprises one or more reagents for use in a process utilizing one or more of the elements described herein. Reagents may be provided in any suitable container. For example, a kit may provide one or more delivery or storage buffers. Reagents may be provided in a form that is usable in a particular process, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more of the vectors, proteins and/or one or more of the polynucleotides described herein. The kit may advantageously allow the provision of all elements of the systems of the invention. Kits can involve vector(s) and/or particle(s) and/or nanoparticle(s) containing or encoding RNA(s) for 1-50 or more neoantigen mutations to be administered to an animal, mammal, primate, rodent, etc., with such a kit including instructions for administering to such a eukaryote; and such a kit can optionally include any of the anti-cancer agents described herein. The kit may include any of the components above (e.g. vector(s) and/or particle(s) and/or nanoparticle(s) containing or encoding RNA(s) for 1-50 or more neoantigen mutations, neoantigen proteins or peptides) as well as instructions for use with any of the methods of the present invention.

In one embodiment the kit contains at least one vial with an immunogenic composition or vaccine. In one embodiment the kit contains at least one vial with an immunogenic composition or vaccine and at least one vial with an anticancer agent. In one embodiment kits may comprise ready to use components that are mixed and ready to administer. In one aspect a kit contains a ready to use immunogenic or vaccine composition and a ready to use anti-cancer agent. The ready to use immunogenic or vaccine composition may comprise separate vials containing different pools of immunogenic compositions. The immunogenic compositions may comprise one vial containing a viral vector or DNA plasmid and the other vial may comprise immunogenic protein. The ready to use anticancer agent may comprise a cocktail of anticancer agents or a single anticancer agent. Separate vials may contain different anti-cancer agents. In another embodiment a kit may contain a ready to use anti-cancer agent and an immunogenic composition or vaccine in a ready to be reconstituted form. The immunogenic or vaccine composition may be freeze dried or lyophilized. The kit may comprise a separate vial with a reconstitution buffer that can be added to the lyophilized composition so that it is ready to administer. The buffer may advantageously comprise an adjuvant or emulsion according to the present invention. In another embodiment the kit may comprise a ready to reconstitute anti-cancer agent and a ready to reconstitute immunogenic composition or vaccine. In this aspect both may be lyophilized. In this aspect separate reconstitution buffers for each may be included in the kit. The buffer may advantageously comprise an adjuvant or emulsion according to the present invention. In another embodiment the kit may comprise single vials containing a dose of immunogenic composition and anti-cancer agent that are administered together. In another aspect multiple vials are included so that one vial is administered according to a treatment timeline. One vial may only contain the anti-cancer agent for one dose of treatment, another may contain both the anti-cancer agent and immunogenic composition for another dose of treatment, and one vial may only contain the immunogenic composition for yet another dose. In a further aspect the vials are labeled for their proper administration to a patient in need thereof. The immunogen or anti-cancer agents of any embodiment may be in a lyophilized form, a dried form or in aqueous solution as described herein. The immunogen may be a live attenuated virus, protein, or nucleic acid as described herein.

In one embodiment the anticancer agent is one that enhances the immune system to enhance the effectiveness of the immunogenic composition or vaccine. In a preferred embodiment the anti-cancer agent is a checkpoint inhibitor. In another embodiment the kit contains multiple vials of immunogenic compositions and anti-cancer agents to be administered at different time intervals along a treatment plan. In another embodiment the kit may comprise separate vials for an immunogenic composition for use in priming an immune response and another immunogenic composition to be used for boosting. In one aspect the priming immunogenic composition could be DNA or a viral vector and the boosting immunogenic composition may be protein. Either composition may be lyophilized or ready for administering. In another embodiment different cocktails of anti-cancer agents containing at least one anti-cancer agent are included in different vials for administration in a treatment plan.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### An Efficient Sample Processing and Analysis Pipeline for HLA-Peptide Sequencing

In this study, Applicants develop a biochemical and computational pipeline for mass spectrometric (MS) analysis of peptides bound to HLA to identify the universe of endogenously presented peptides and improve our understanding of the rules governing antigen presentation. Applicants focused the analysis on single HLA class I allele-expressing cell lines, so motifs could be assigned to alleles unambiguously (12, 13). The studies leveraged advances in instrumentation for rapid collection of high resolution data and database search tools that consider HLA peptide-binding motifs integrated with proteogenomic analysis strategies (14). Herein, Applicants combine these improvements to comprehensively evaluate the characteristics of HLA-associated peptides presented by 16 HLA alleles with the goal of improving the performance of prediction algorithms for class I HLA peptide-binding.

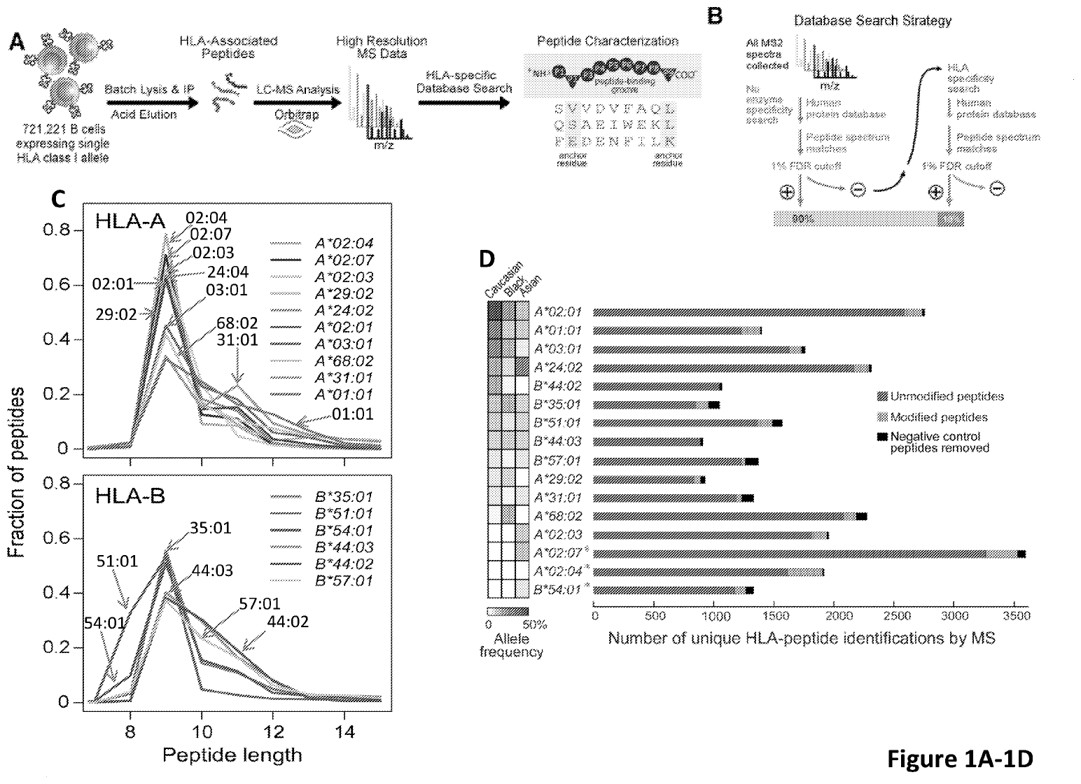

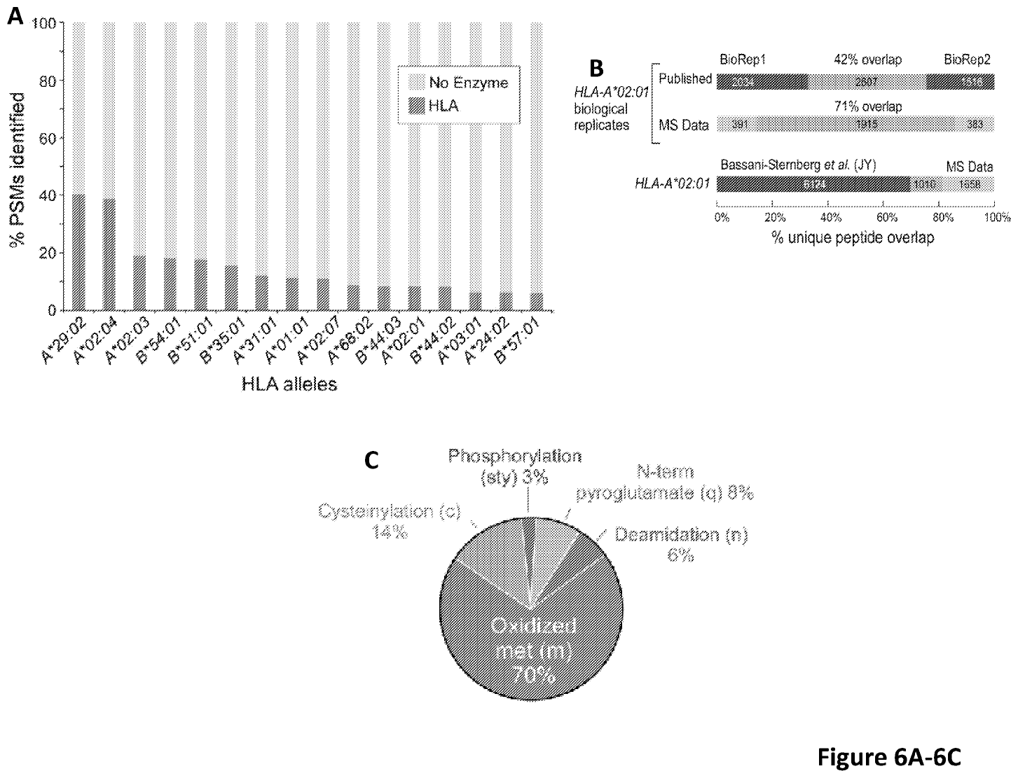

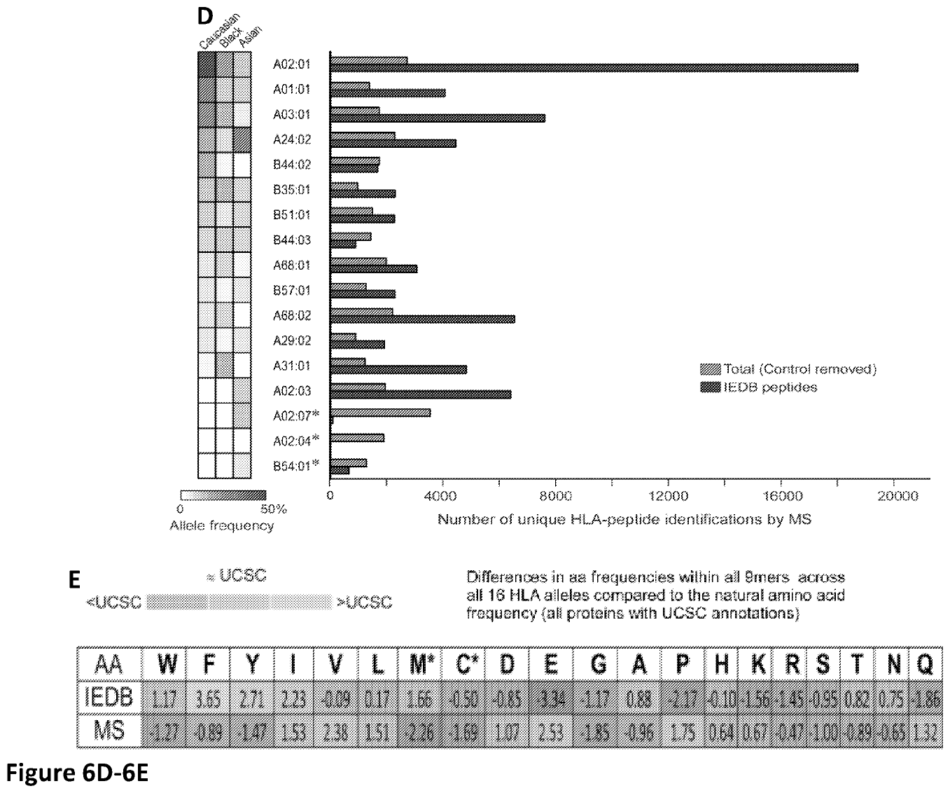

Applicants immunoaffinity-purified and sequenced HLA-associated peptides from 30-90 million cells of class I deficient B cell lines (B721.221) stably transduced to express a single class I HLA allele (FIG. 1A). These alleles were selected from Caucasian, Black, and Asian populations because they were understudied or for their disease associations(15-17). High quality tandem mass spectra (MS/MS) were subjected to iterative database searches where stringent criteria were applied for precursor ion purity and allowable percentage of unassigned ions in the MS/MS (Materials and Methods). The first search round used no enzyme specificity and no variable peptide modifications, while the second round applied an HLA-specific enzyme specificity based on first-round results and allowed peptide modifications (FIG. 1B). The second round of search typically increased identifications by an average of 14% (5-40%) while maintaining a stringent 1% FDR cutoff. Peptide spectrum matches (PSMs) passing a stringent <1% FDR estimation cutoff from both search rounds were combined and reported for each HLA allele (FIG. 1D, FIG. 6A; Table 1A).

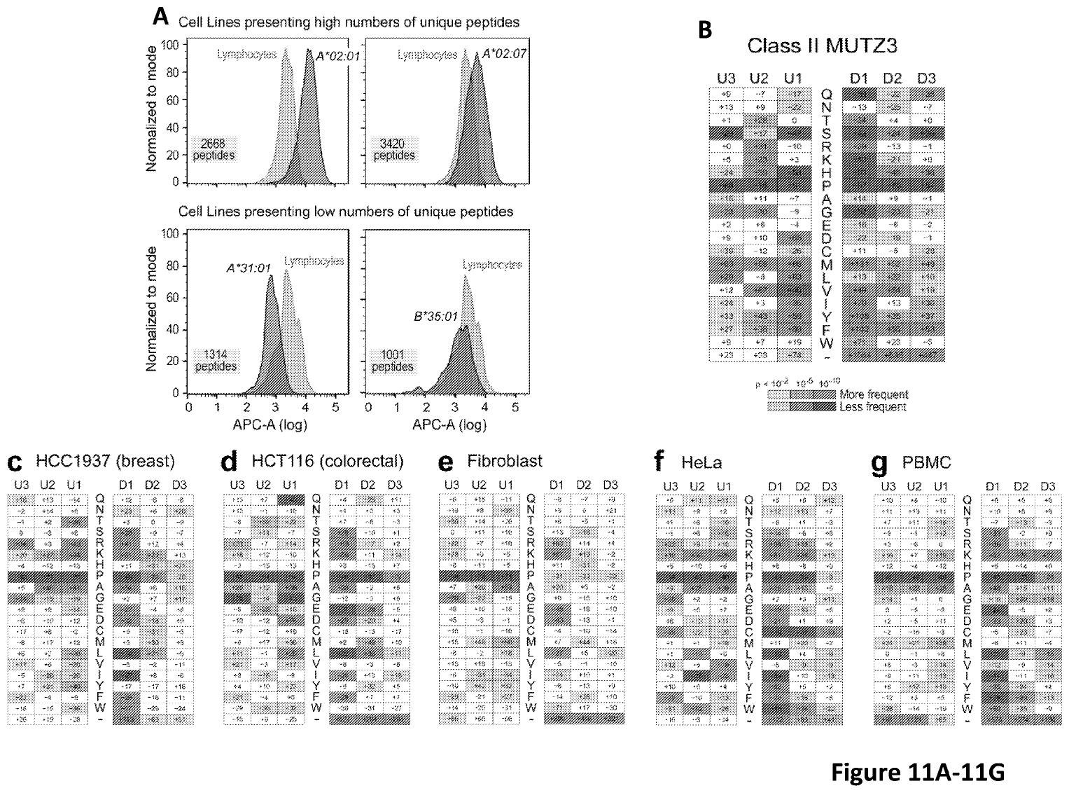

Non-specifically-bound peptides (negative controls) were identified by immunopurification of untransduced B721.221 cells and B cells processed with beads not conjugated with the pan-class I HLA binding antibody (W6/32) (Table 1A, FIG. 1D). Approximately 3% (σ3%) of all peptide identifications were shared with the pool of 223 negative control peptides. After filtering for these non-specific binders, between 900 and 3550 unique peptides were identified by LC-MS/MS for each HLA allele (median 1505), with length distributions matching the expected 8-15 amino acids (FIG. 1C). All peptides identified in the negative controls were subtracted prior to motif determinations and further analyses. For the 14 alleles with frequencies in Caucasians of greater than 1%, our LC-MS/MS-based workflow yielded a median of 49% (range 15-100%) of the number of peptides existing in IEDB. For HLA-B*54:01, HLA-A*02:07 and HLA-A*02:04, with minor allele frequencies of less than 1% within Caucasian populations, 2-, 40- and 450-fold more peptides, respectively, were identified compared to IEDB (FIG. 6D). Variation in surface presentation of HLA molecules on B721.221 cells, as compared to primary lymphocytes, appeared to explain most of the variation in observed peptide counts (FIG. 11A). For common alleles (population frequency >1%), 74% of peptides were not reported in the immune epitope database (IEDB); for rare alleles, nearly 100% were unreported (Table 1D).

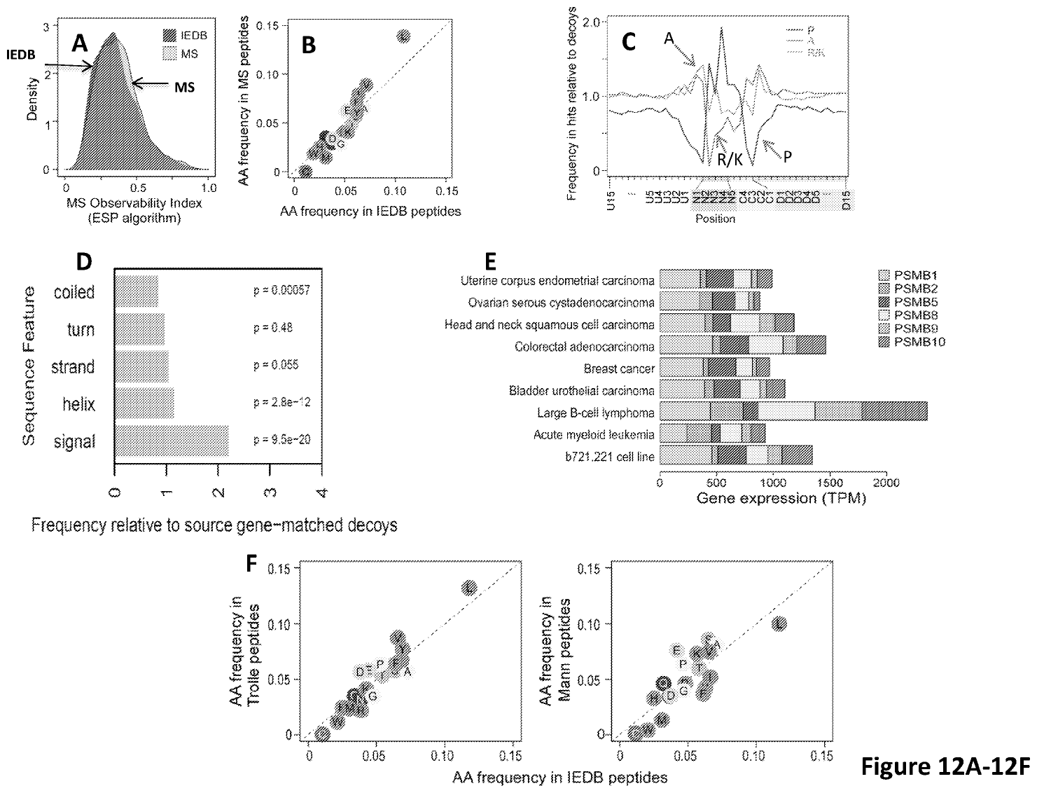

A high degree of peptide overlap was observed between biological replicates (˜70%) and a published B cell HLA-peptide dataset (1) (FIG. 6B). A median of 92% of presented peptides were unmodified (σ=5%), while only a median of 4-5% (σ=4%) were modified. Of the modified peptides identified, most were consistent with frequently observed artifacts of sample handling (70% with oxidized methionine, 6% with deamidation, and 8% with pyroglutamic acid at the N-terminus), while 3% contained phosphorylation, an endogenous post-translational modification (FIG. 6C) (18). There were only negligible peptide sequence biases related to the experimental procedures based on comparisons among MS peptides and allele-matched synthetic peptides that were assigned as binders by IEDB (measured affinity <500 nM; FIGS. 12A and 12B). The predicted MS observability of the HLA peptides and frequencies of individual amino acids between MS and IEDB peptides were highly similar, aside from underrepresentation of cysteine (FIGS. 12A and 12B). Free cysteine, which interferes with precursor fragmentation during LC-MS/MS, is underrepresented in other MS-based HLA-peptide datasets (1). Cysteine-containing peptides were recovered when a third round of database search accounted for cysteinylation (Table 5).

### Example 2

### Novel HLA Peptide-Binding Motifs Enriched in LC-MS/MS Data Relative to IEDB

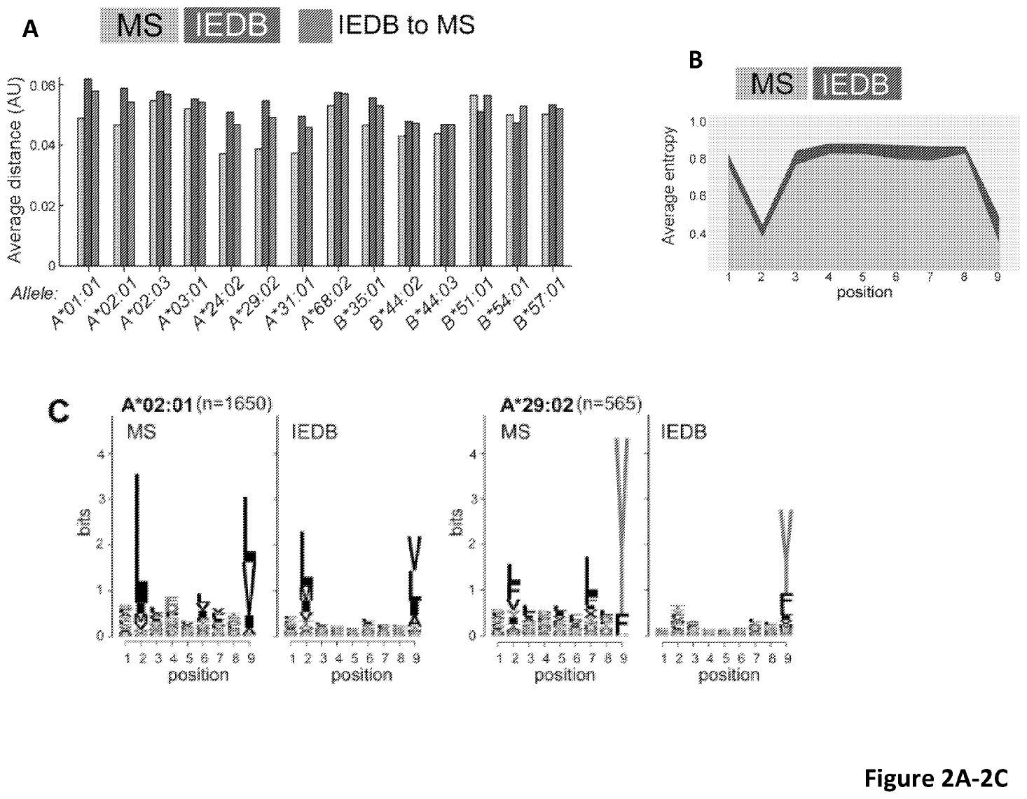

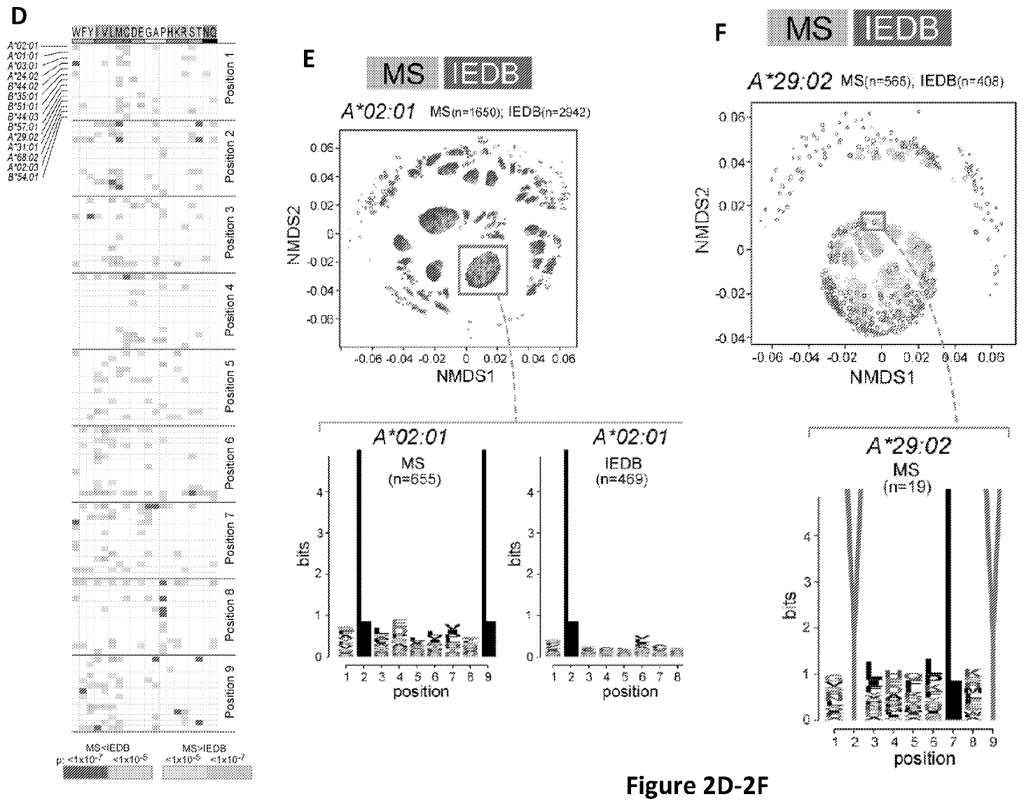

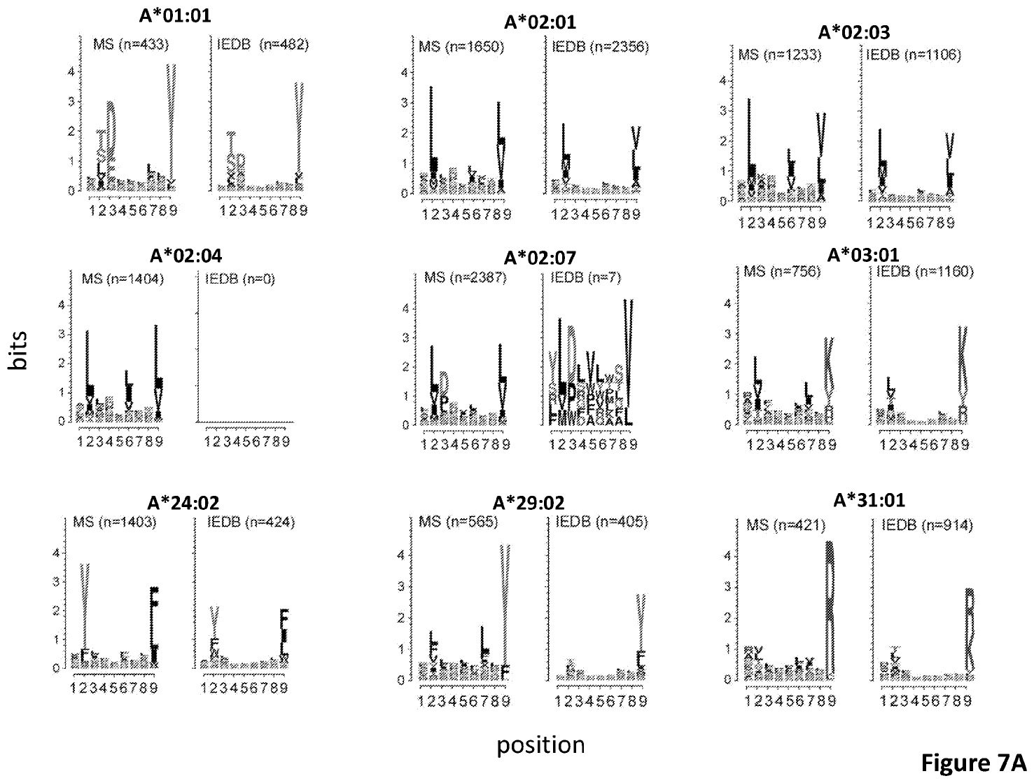

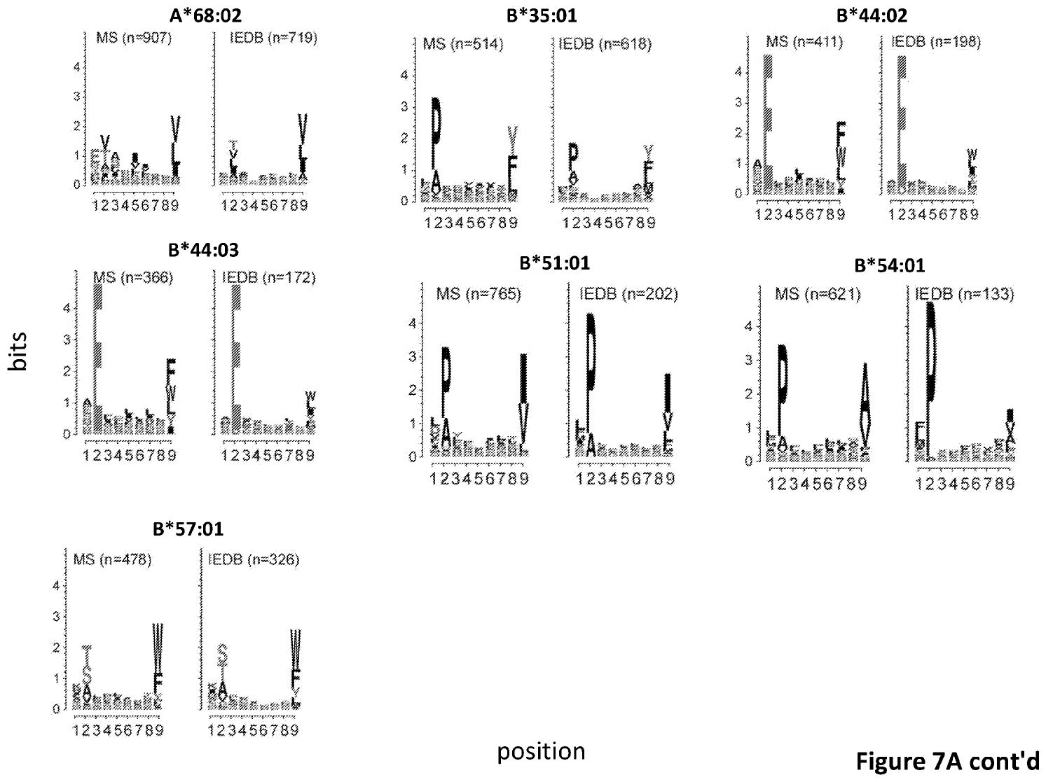

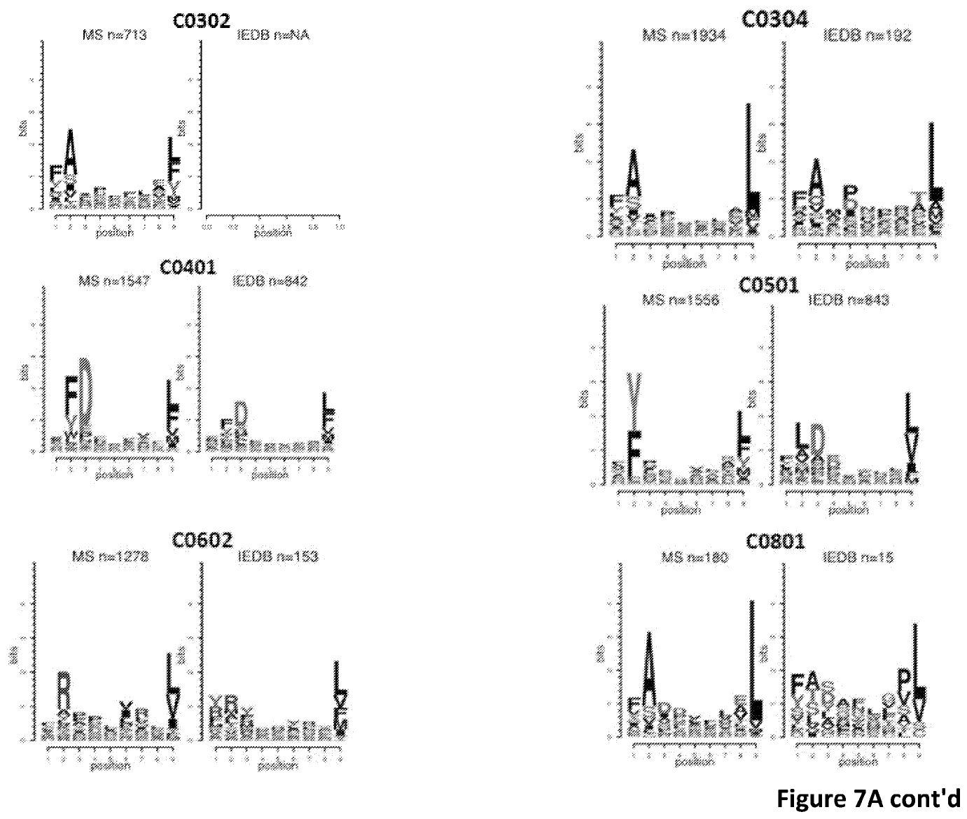

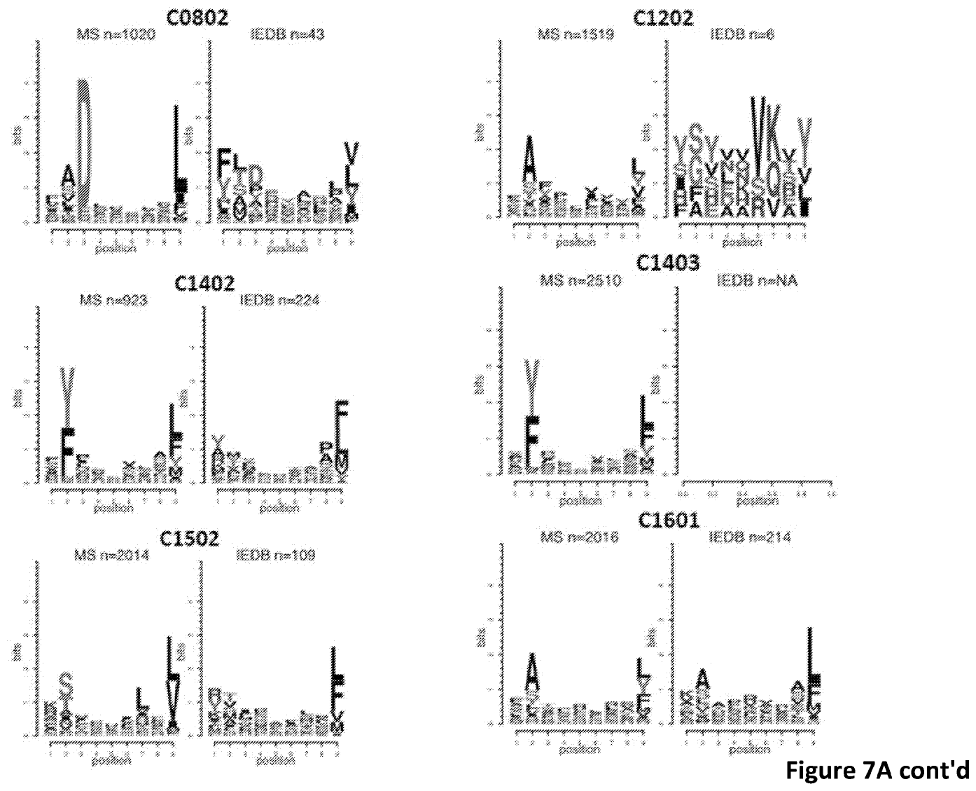

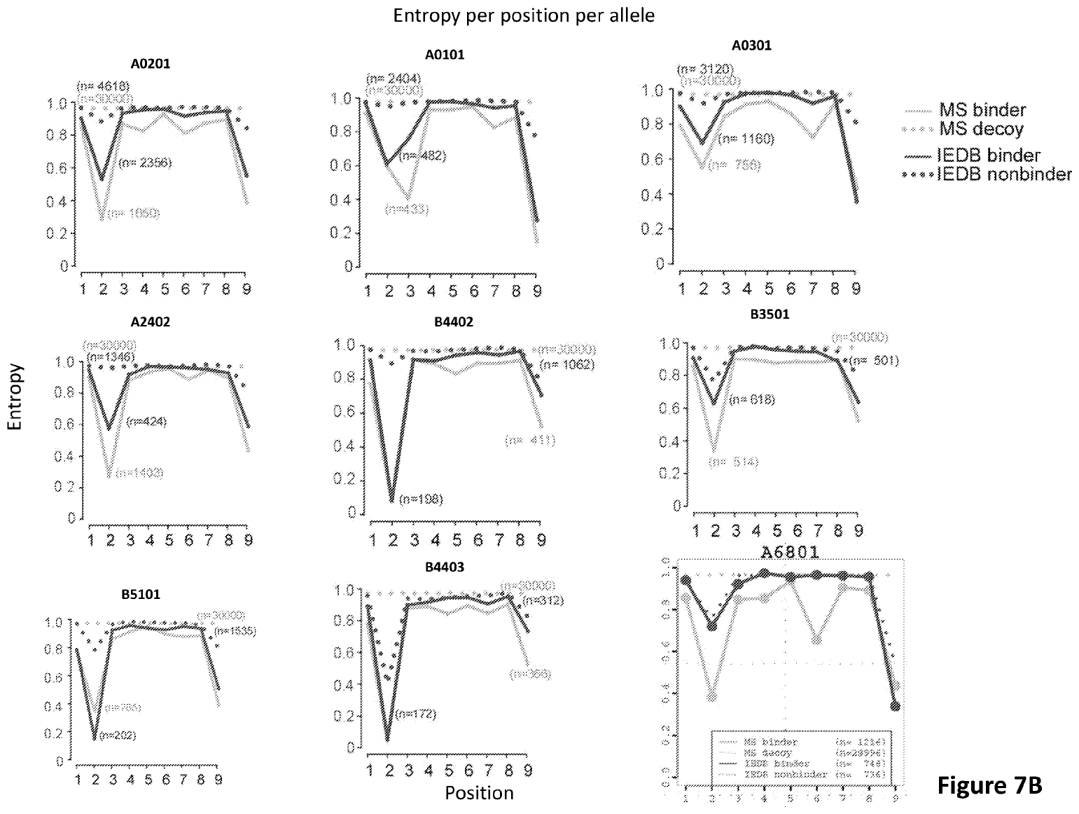

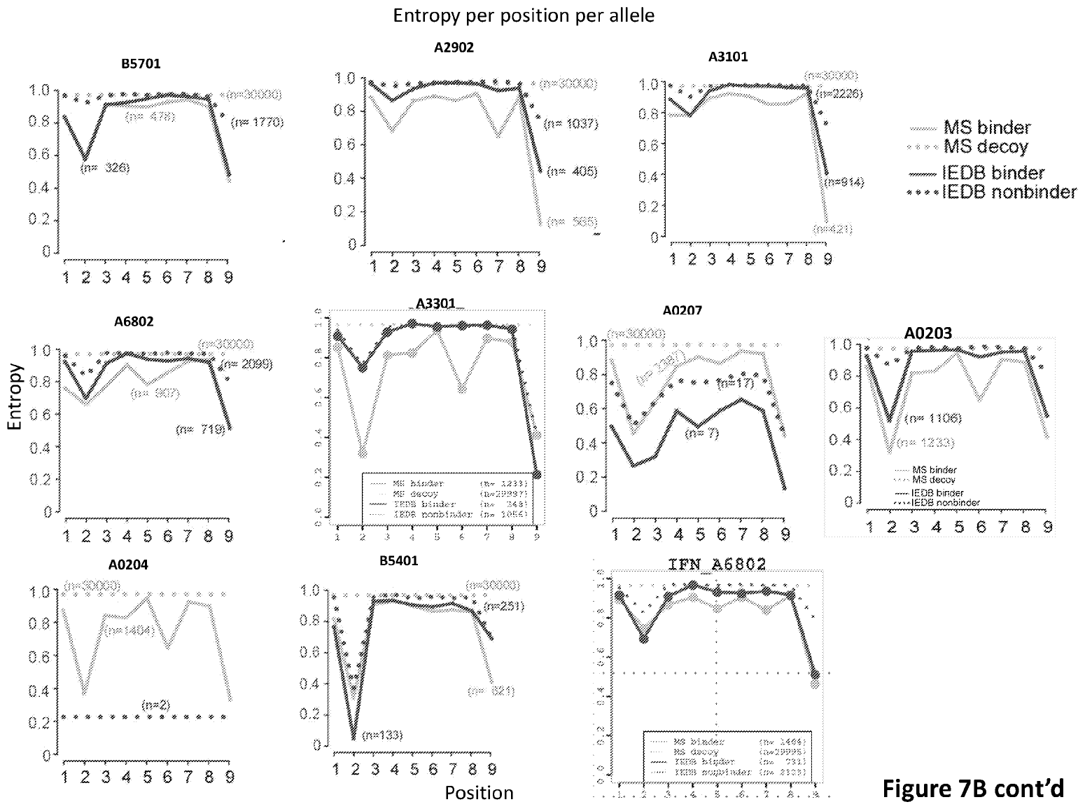

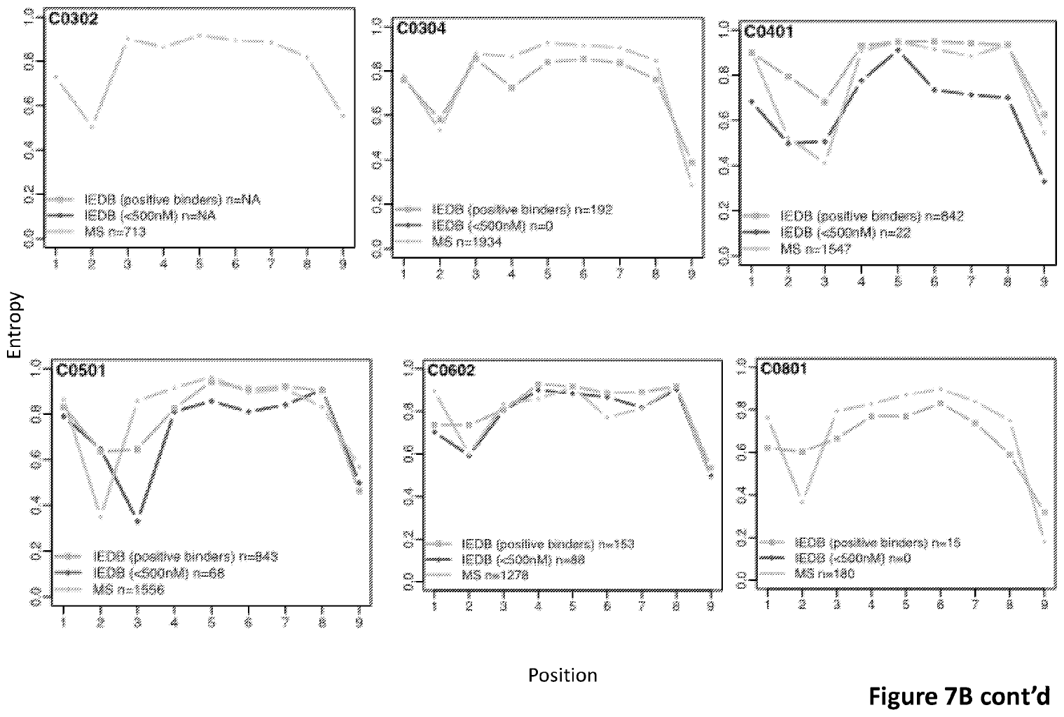

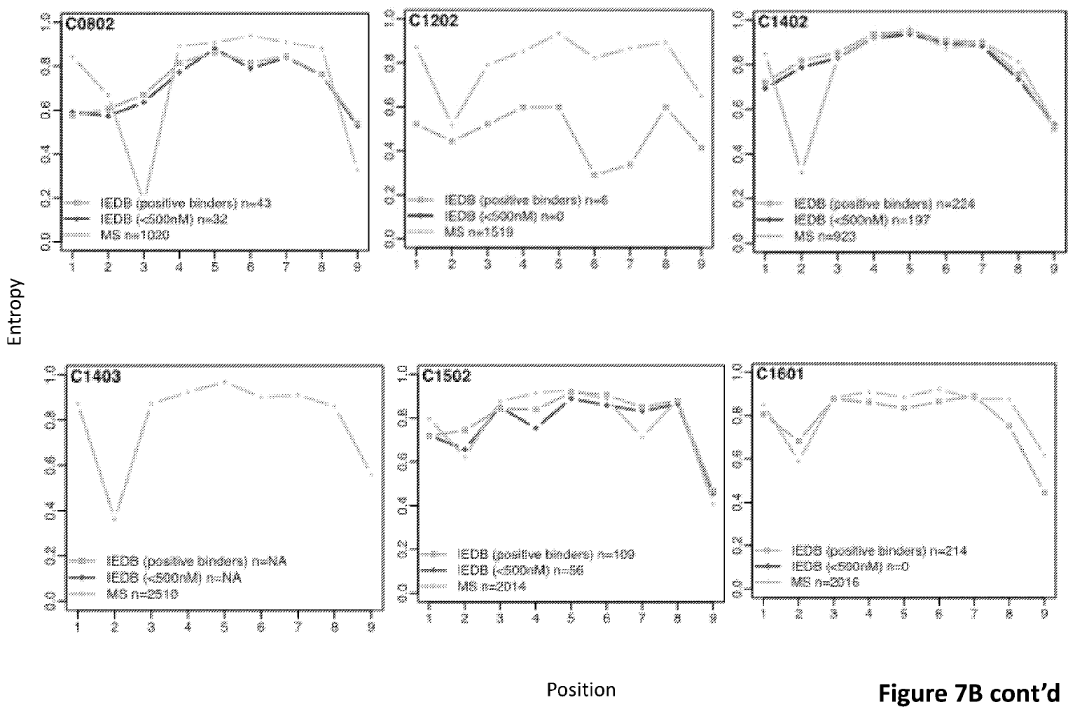

Comparison of MS and IEDB peptides showed significant differences in amino acid frequencies at specific positions. Assessment of entropy at each position within 9mers of LC-MS/MS and IEDB datasets (FIG. 2A) revealed the lowest average entropy (<0.4) at the positions 2 and 9 anchors, while low entropy was also observed for positions 3 through 7 mainly in the LC-MS/MS data with variation amongst alleles (FIG. 7B). For example, the HLA-A*02:01 data uniquely revealed sub-anchors at positions 4 (E, D), 6 (L, V, I), and 7 (I, V, A) (FIG. 2B-left). Likewise, in the example of HLA-A*29:02 and other alleles, evidence of enriched residues at positions consistent with secondary anchors were observed (FIG. 2B-right; FIG. 7A). More than 11% of 2340 possible changes (20 AAs*13 alleles*9 positions) were significantly different (FIG. 2C). Methionine (M), cysteine (C), and tryptophan (W) were over-represented in IEDB peptide sequences (p<1×10⁻⁵, chi-square test) while the amino acids isoleucine (I), valine (V), and leucine (L) (p<1×10⁻⁵, chi-square test) were under-represented, especially at positions 5-7 that encompass secondary anchors. This was true for both sparsely studied alleles, like HLA-A*02:07, and for well-studied alleles like HLA-A*68:02 and HLA-B*57:01. Applicants also noted specific alleles with length preferences not captured in IEDB, such as HLA-A*31:01 and HLA-B*51:01, which bind high proportions of 11mers and 8mers, respectively (FIG. 1C).

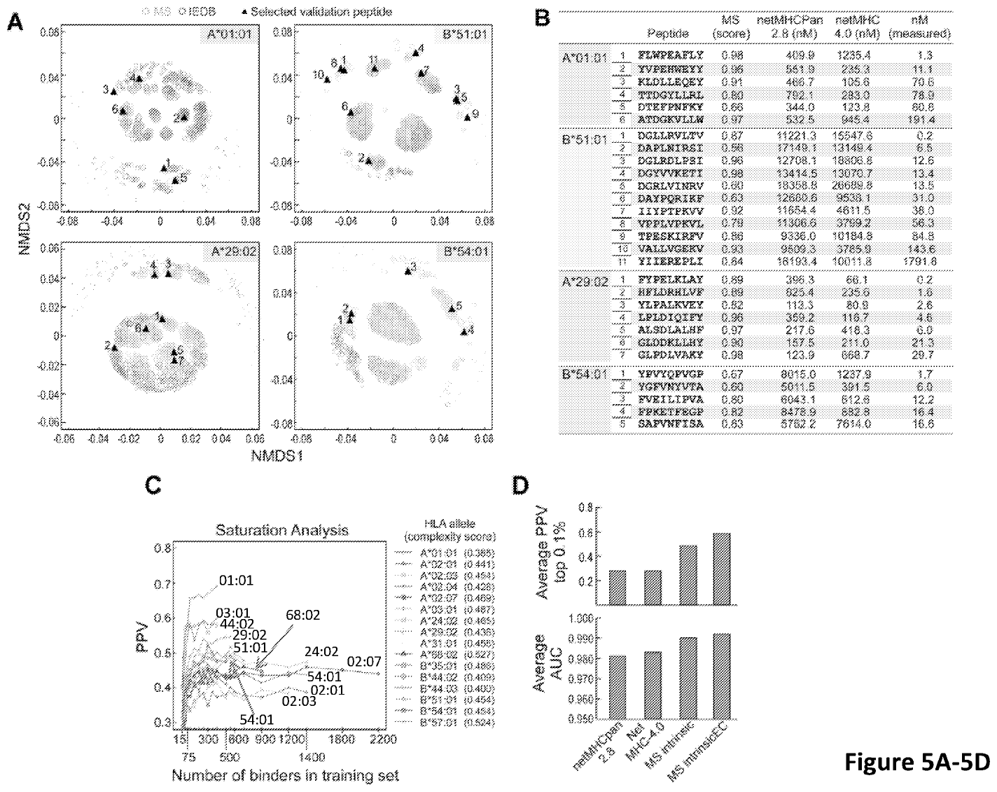

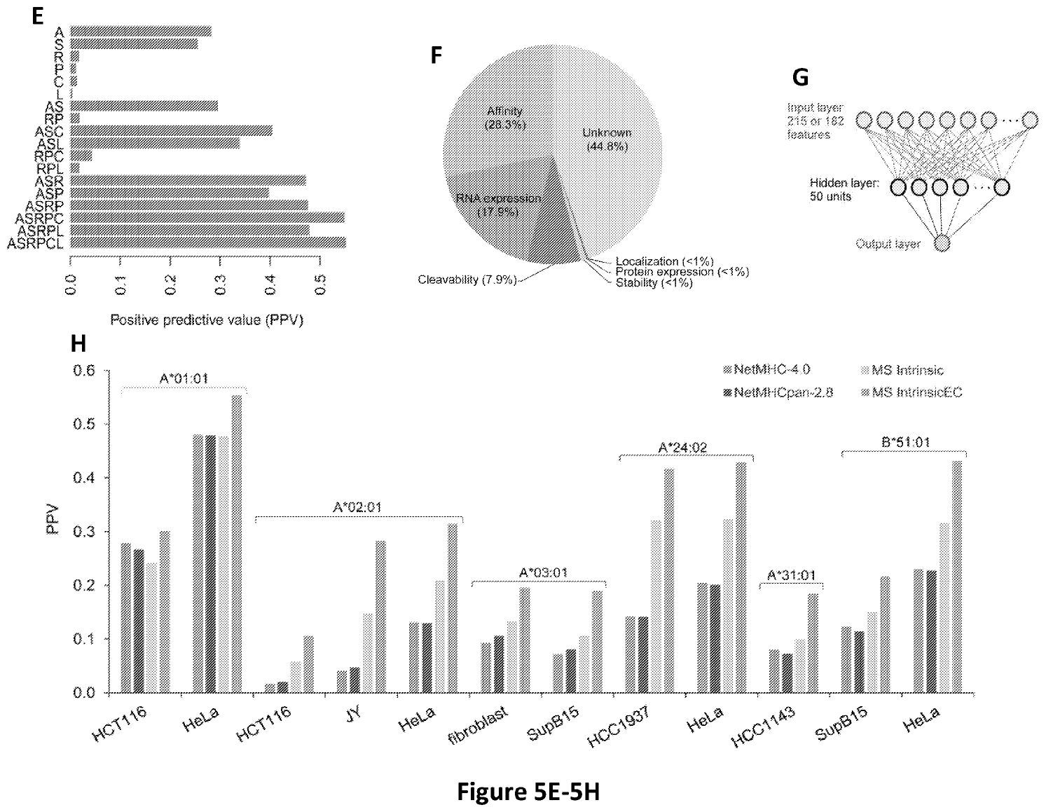

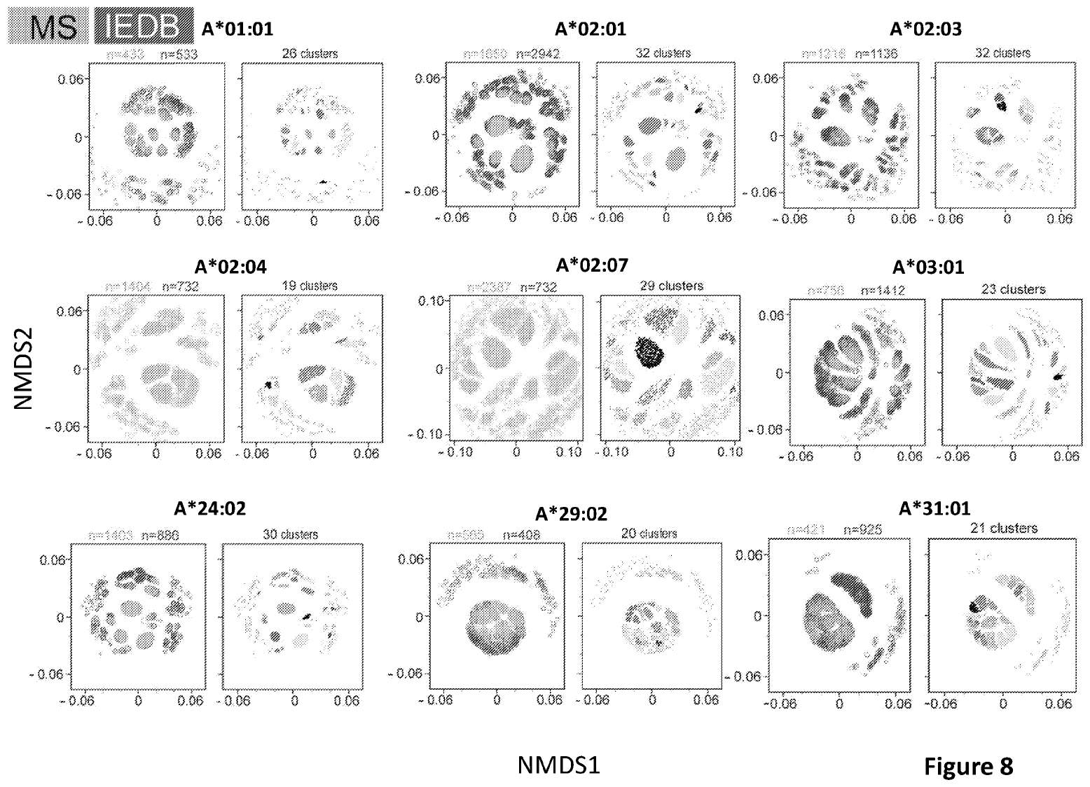

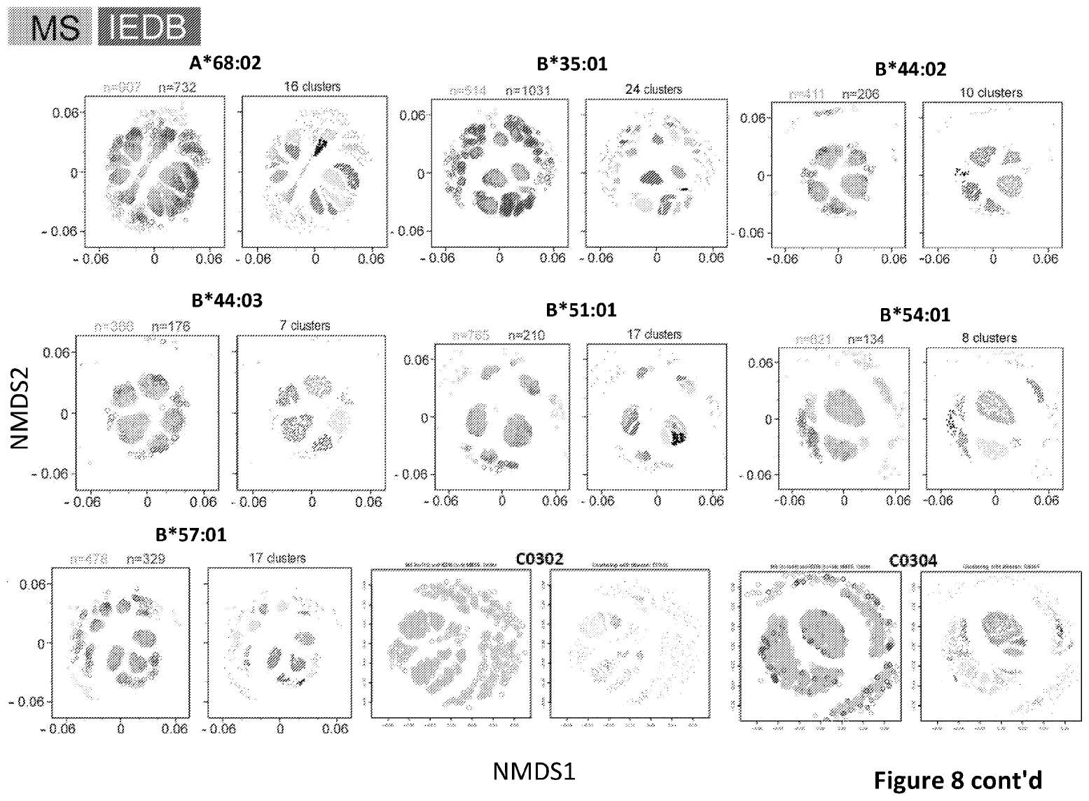

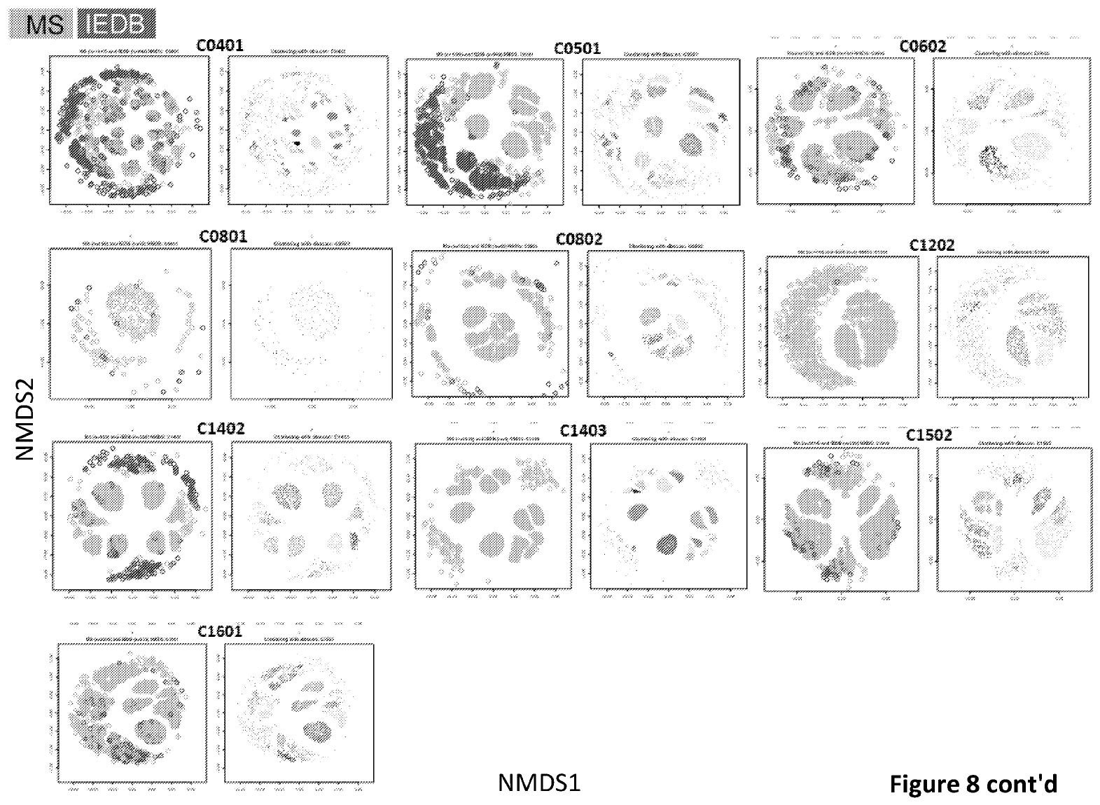

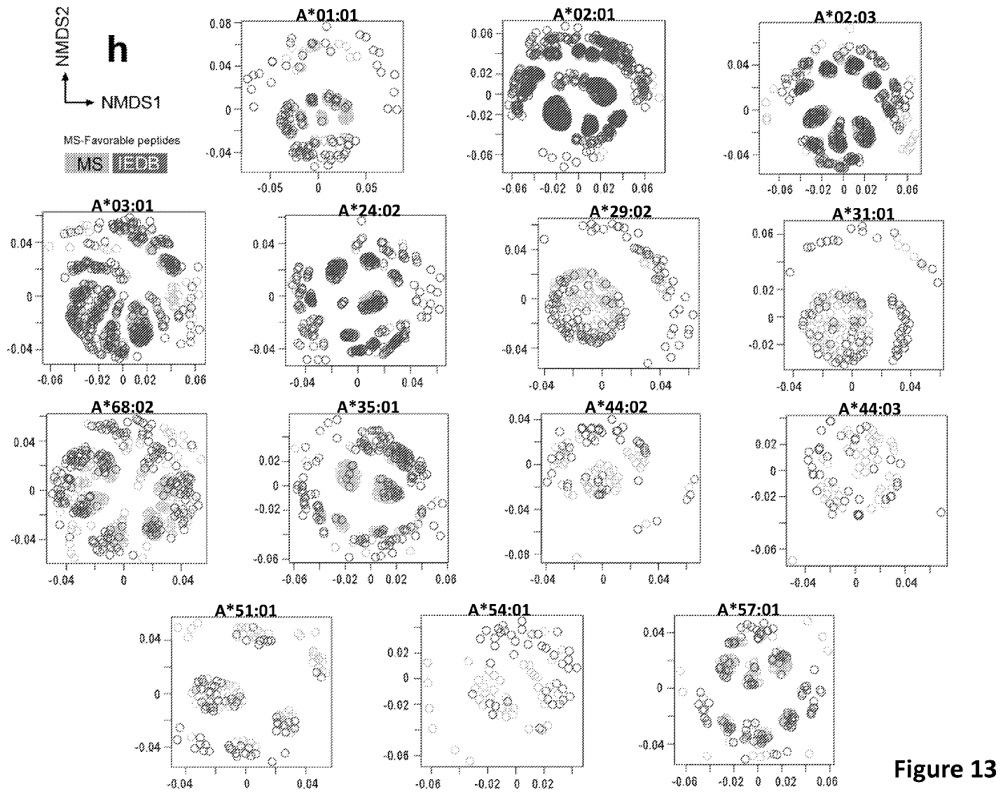

The 9mer peptides bound to a particular HLA allele were systematically compared to peptides reported in the IEDB database for the same allele by computing a distance metric. Applicants devised a metric that does not weight each position equally since some positions are more critical for binding HLA than others. Applicants defined an entropy-weighted peptide distance and plotted the peptides in two-dimensional space such that “similar” peptides would be clustered closely and dissimilar peptides distantly (see FIGS. 5A and 8). For positions with reduced entropy (i.e., fewer possible residues; FIG. 2A), Applicants increased the weight of that position in the distance calculation. The distance was calculated using a pre-calculated matrix of similarities between residues, biased by their HLA binding properties (19). Based on entropy-weighted distance, the peptides identified per HLA by MS were typically closer to each other than to peptides in IEDB; MS peptides were also closer to each other than IEDB peptides were to themselves (FIG. 7B), suggesting that MS recovers stronger binding motifs compared to a greater preponderance of weak binding peptides in the IEDB binder sets. Moreover, Applicants found multiple peptide clusters that were highly enriched in MS relative to IEDB (FIGS. 2E and 2F), reflecting unique information in the MS datasets. MS technology-related biases did not appear to underlie these patterns, as a similar analysis focused on only the subset of peptides from MS or IEDB with physicochemical properties favorable for detection by MS revealed similar distances and clustering patterns (FIGS. 13 and 2A) (Eyers et al., 2011, Mol Cell Proteomics (2011); 10(11):M110.003384; Fusaro et al., 2009, Nature Biotechnology 27, 190-198; Muntel et al., 2015, Mol. Cell. Proteomics 14, 430-440; Searle et al., 2015, Mol. Cell. Proteomics 14, 2331-2340). Applicants then visualized these peptides in clusters using a non-metric multidimensional scaling (NMDS) based on the distance metric, and observed that MS data tended to cluster more closely together in a compared to IEDB peptides. For the well-characterized HLA-A*02:01 allele, the LC-MS/MS and IEDB datasets generated a high degree of overlap in peptide clusters, with similar pairwise distances among 9mers that had measured affinities <500 nM (FIG. 2D). For most alleles, however, several peptide clusters highly enriched by LC-MS/MS data were revealed, demonstrating the extent to which new classes of binding peptides were discoverable (FIG. 8). Peptide clustering was driven by the amino acids with the lowest entropy (i.e. anchor residues) due to the entropy-weighted distance; for example, tyrosine (Y) was determined to be a position 2 anchor of HLA-A*29:02-bound peptides, which dominated the cluster highlighted in FIG. 2E.

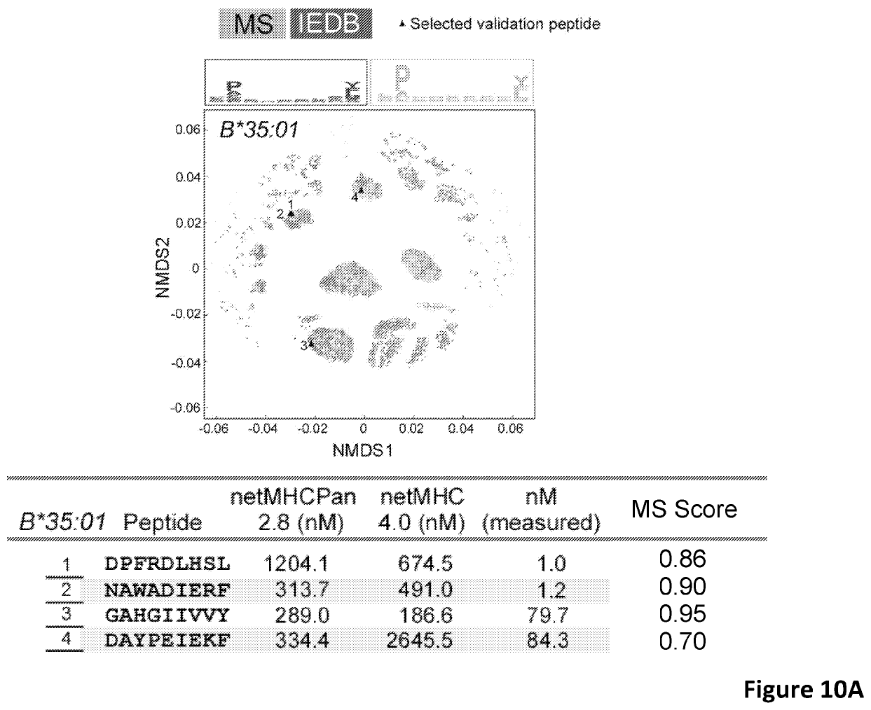

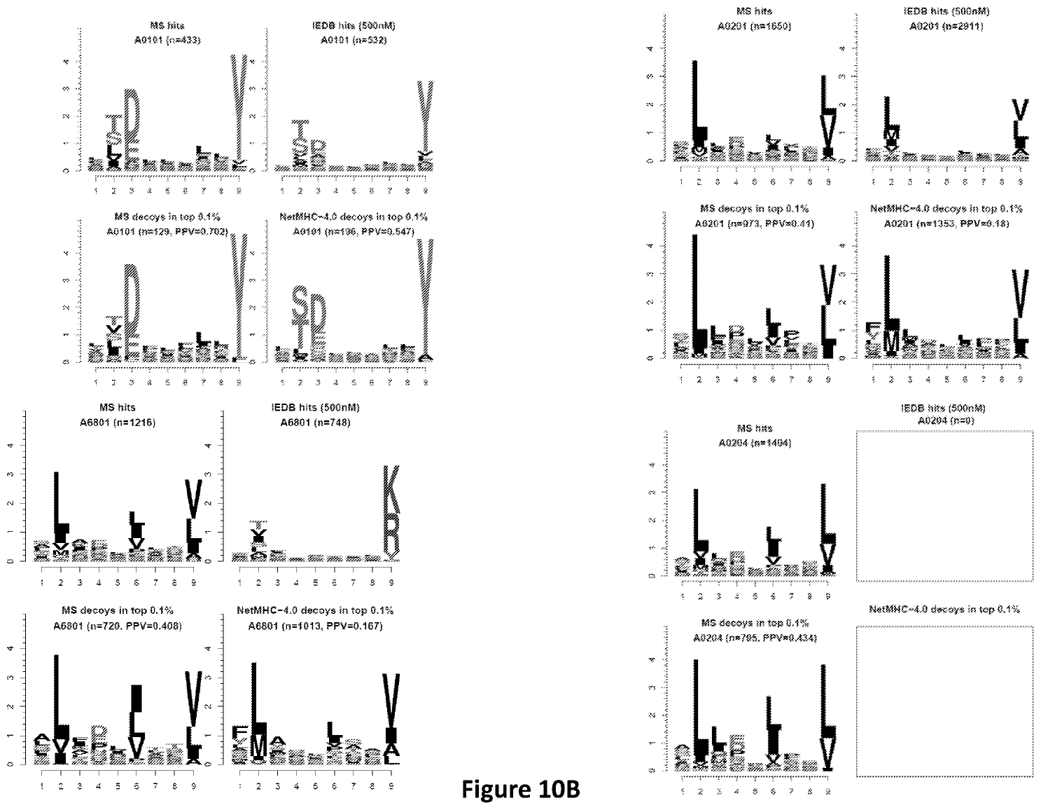

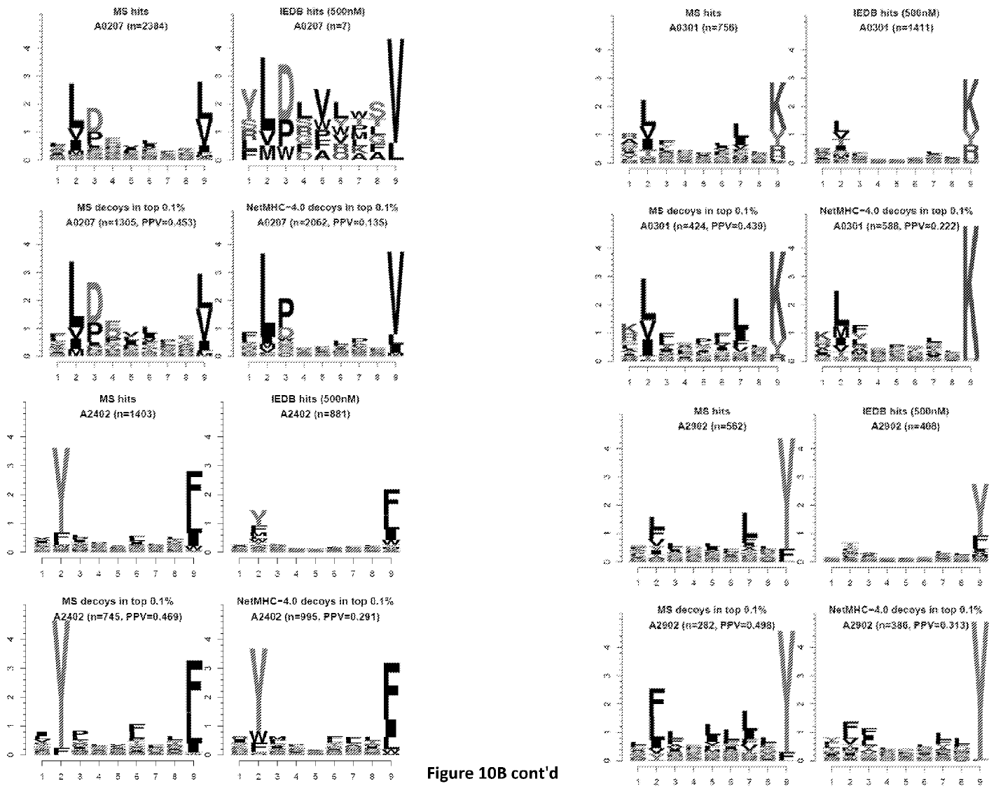

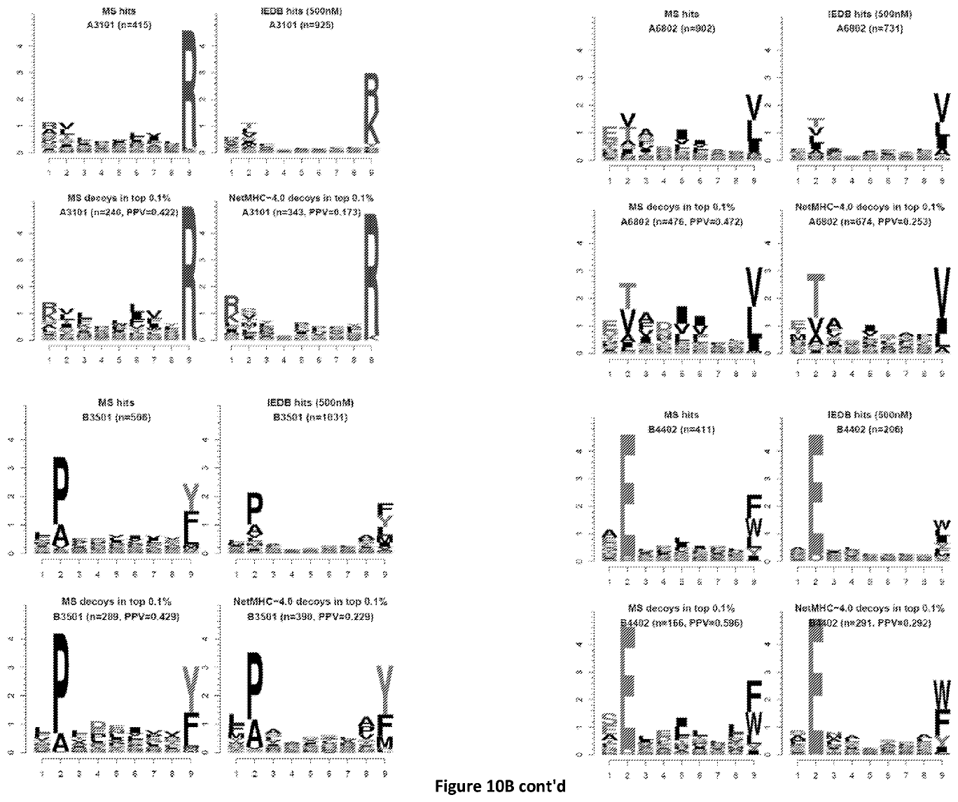

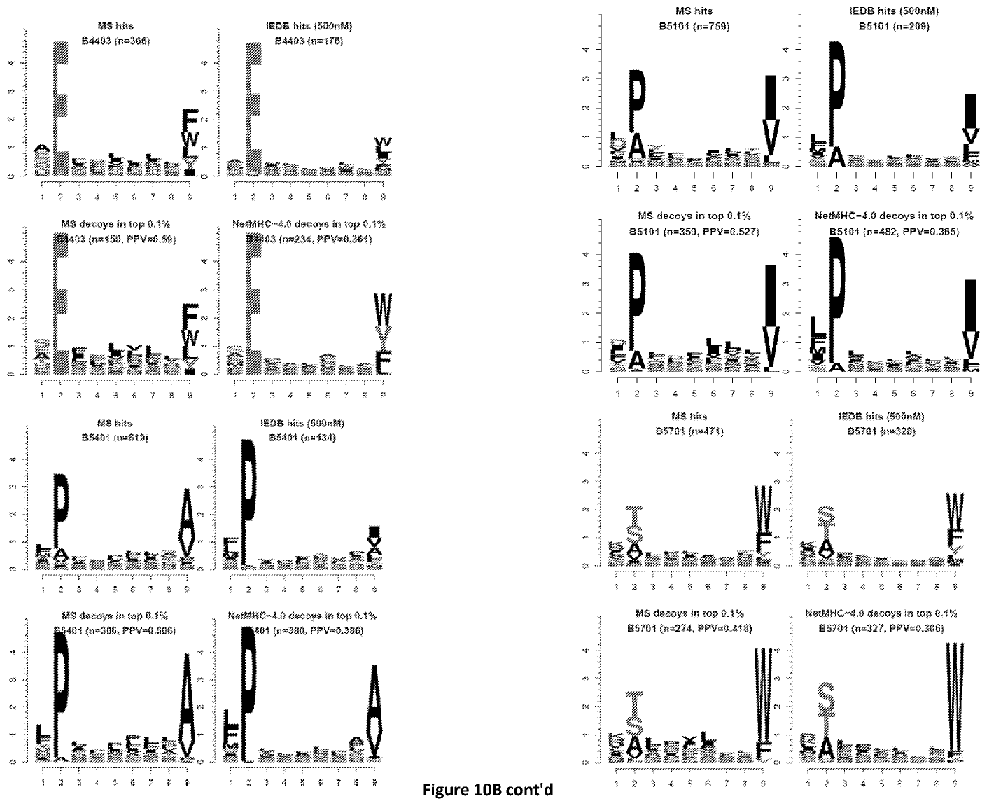

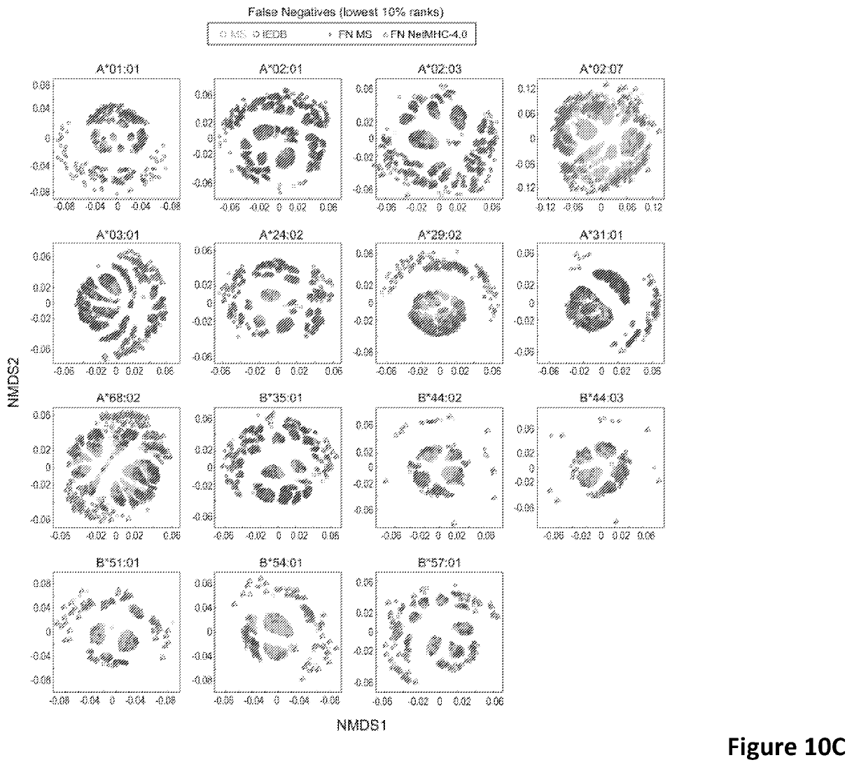

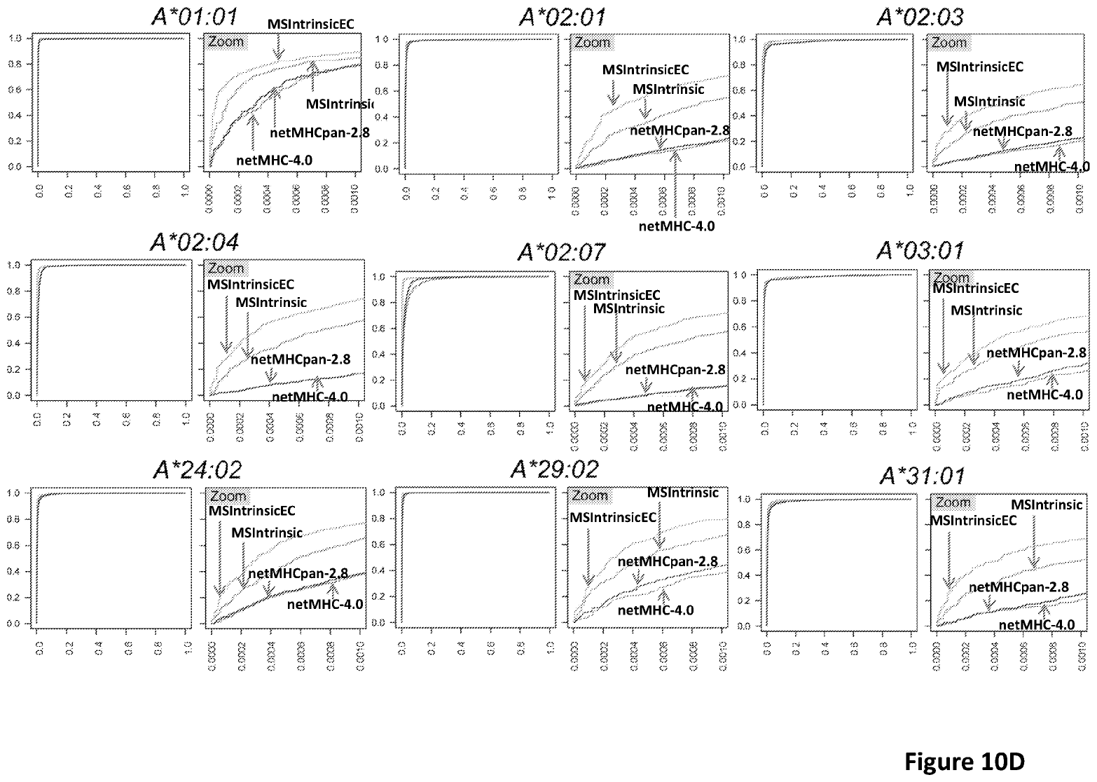

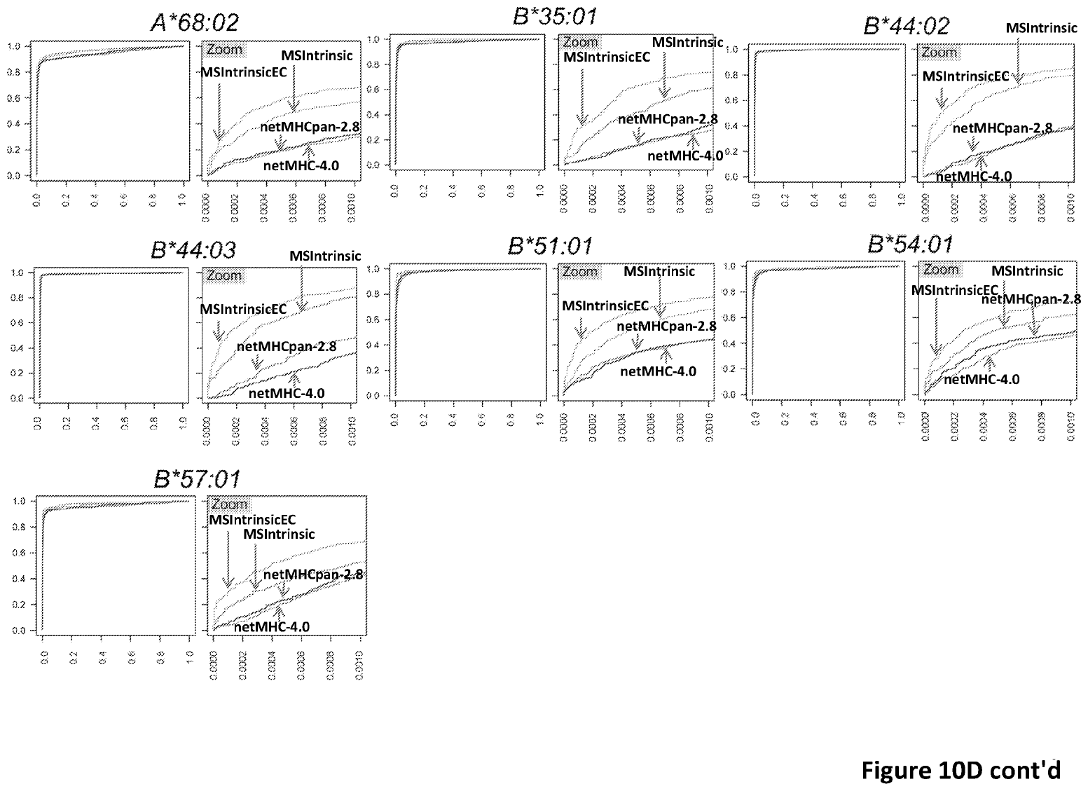

To validate identified motifs, Applicants selected sequences from clusters enriched within the MS datasets but scored only within the bottom 10% when MS hits were evaluated by NetMHCpan-2.8. By competitive peptide-binding assays, 32 of 33 peptides were confirmed to be strong binders (median IC50<14 nM), despite only 14 of 33 having been predicted to be binders (<500 nM) by NetMHCpan-2.9 (FIGS. 5A, 5B, and 10A).

### Example 3

Novel Insights into Endogenous Antigen Processing and Presentation Yielded by the LC-MS/MS Data.

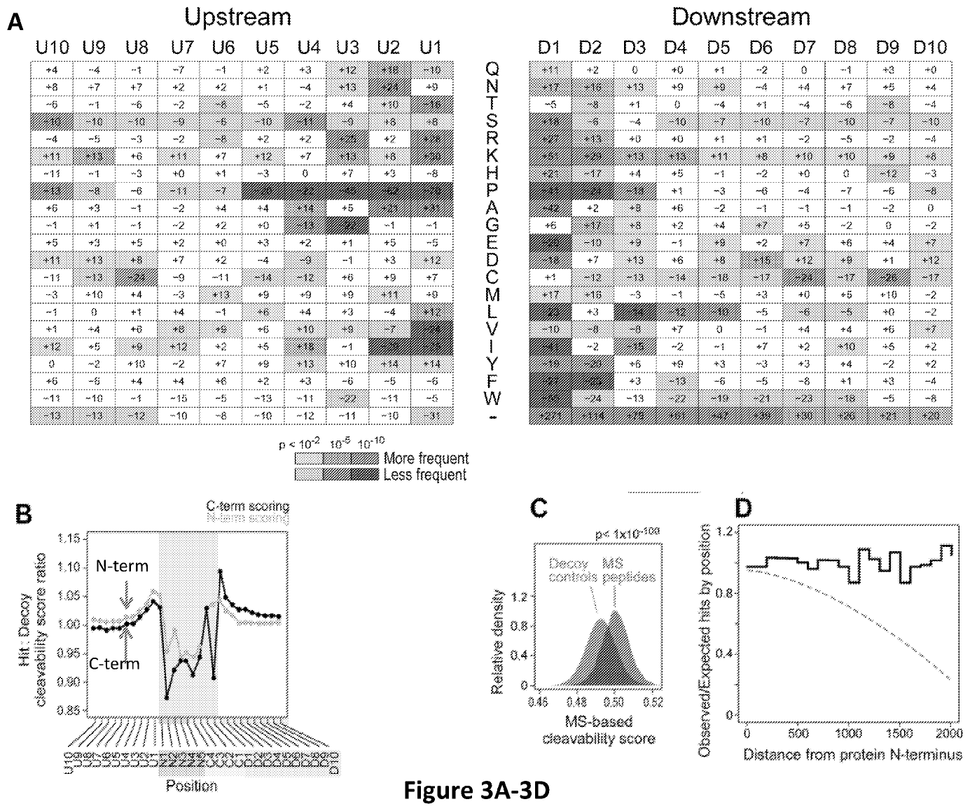

Applicants analyzed a large data set of 24,000 allele-specific MS peptide and found motifs in the upstream and downstream flanking sequences, as well as within the HLA-binding peptide. Applicants focused on the sequence context around each HLA-peptide within its source protein, which is not confounded by HLA binding (FIG. 3A). Applicants systematically examined the specificity of proteasomal cleavage by determining the frequencies of amino acids upstream and downstream of the N- and C-termini of all peptides sequenced by LC-MS/MS. At both the N- and C-terminus, an enrichment in lysine (K) and arginine (R), consistent with the tryptic-like specificity of constitutive proteasome subunits was observed (20) (FIG. 3A). For example, upstream of the peptide, at the first position (“U1”), arginine and lysine were highly enriched (relative to peptide decoys, consisting of random proteome 9mers matched for their first two and last two amino acids), indicating a strong trypsin-like specificity at the N-terminus (FIG. 3A). Downstream of the peptide, arginine and lysine were also enriched in the first position (“D1”), (suggesting that peptides are trimmed at the C-terminus after a tryptic-like cleavage that occurs after these basic residues), and acidic residues were depleted in this position. Enrichment for alanine (A), particularly at the U1 and D1 position, and an under-representation or strong depletion of proline (P), extending 3-5 residues upstream and downstream, which may related to proline's regid peptide bonds, were observed. In addition, there was a strong preference for peptides arising at the C-terminus of their source protein (laelled as “-” in FIG. 3A, signifying empty position), where only a single cleavage event is required. While HLA-binding motifs hamper the discovery of cleavability signatures within MS-identified peptides, Applicants determined whether the cleavability signatures observed at the N- and C-termini were depleted within peptide sequences. To this end, two indices for residue cleavability (“N-terminal scoring” and “C-terminal scoring”) were applied to each position upstream, downstream, and internal to the MS-observed peptides. Comparing against a set of 1×10⁶ 9mers randomly drawn from the genome, a significant reduction in cleavability was detected within the internal peptide sequence, as hypothesized (FIG. 3B). Furthermore, cleavability was most enriched at the C-terminus. This pattern is consistent with existing models of peptide processing, wherein the C-terminus is determined by the proteasome, and the N-terminus is determined not only by proteasomes, but also by cytosolic proteases, or ERAP1/2 trimming (21, 22). By comparing amino acid frequencies upstream, within, and downstream of each peptide, Applicants also observed depletion of “cleavable” amino acids (K, R, and A) and enrichment of “non-cleavable” proline within peptides (FIG. 12C). Thus, avoidance of internal cleavage appears to be a key feature of HLA ligands. Applicants also considered whether protein sequence features, such as alpha helices and beta strands, might influence processing potential (FIG. 12D). LC-MS/MS peptides were twice as likely as gene-matched decoys to arise from signal peptide sequences; other features were significant but did not show effect size greater than ±15%.

To explore whether the processing signature was likely to be generalizable, Applicants analyzed the gene expression of a proteasome and the immunoproteasome; both were expressed in B721.221 B cells at proportions comparable to those in blood and epithetial cancers included in the cancer genome atlas (TCGA) (FIG. 12E). When Applicatns examined the HLA-bound peptide repertoires previously recovered from cells of other lineages, including breast and colon cancer cells (Bassani-Sternberg et al., 2015, Mol. Cell. Proteomics 14, 658-673 (2015)), fibrolasts (Bassani-Sternberg et al., 2015, Mol. Cell. Proteomics 14, 658-673 (2015)), HeLa cells (Trolle et al., 2016, J. Immunol. (2016), doi:10.4049/jimmunol.1501721), and peripheral blood mononuclear cells (Caron et al., 2015) (FIGS. 11C-G), all the key features observed for B721.221 cells were likewise consistenly observed for these other cell types. Applying this ame analytic approach to reported class II peptides isolated from dendritic cells (Mommen et al., 2016, Mol. Cell. Proteomics MCP 15, 1412-1423) (MUTZ3 cell line), Applicants observed a starkly different signature exhibiting preference for hydrophobic residues in the D1 position and a lack of the previously observed associations for lysine, arginine, and alanine (FIG. 11B). Applicants also note that the HLA class I signature that Applicants derived only mostly resembled that obtained by comparing peptides with high versus low NetChop scores. Applicants' analyses thus identify a common HLA class I cleavage signature that dramatically differs from that predicted by a widely-used tool.

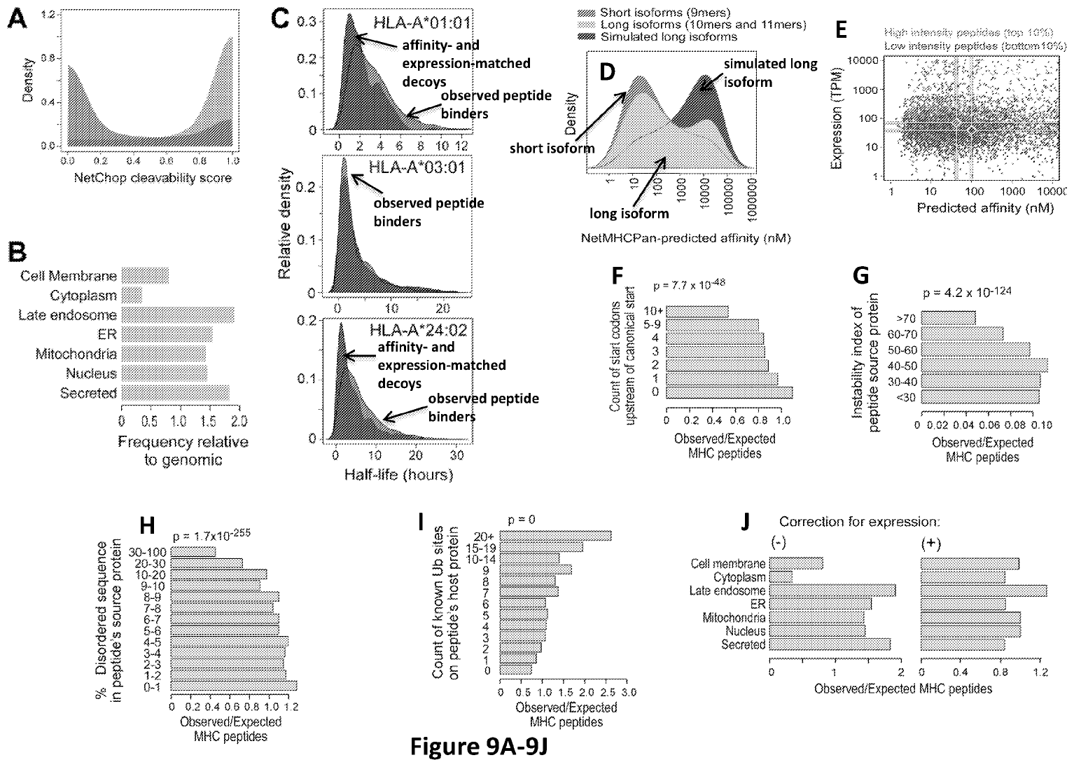

Since cleavability determines availability for HLA-binding, this feature was assessed by scoring peptides with the tool NetChop (23-25). NetChop showed a large difference in cleavage scores when LC-MS/MS-sequenced peptides were compared to 1 million decoy peptides (FIG. 9A). However, this signal was highly allele-dependent and largely mitigated by controlling for predicted binding affinity (Table 4), indicating anchor residue identity as a possible confounding variable. Therefore, an independent cleavage predictor was developed that used the cleavability signatures learned from the N- and C-termini of LC-MS/MS peptides (Methods). This new predictor showed a significant (p=1×10⁻⁸²⁵), but modest divergence between binder and decoy peptides (FIG. 3C) that was consistent across alleles and roughly equivalent to NetChop after controlling for predicted peptide affinity (Table 4). Since HLA-presented peptides have been thought to be products of aborted translation (26, 27) (28), Applicants further determined the positions of all LC-MS/MS-identified peptides within their source proteins. However, there was no evidence that peptide positional frequencies were shifted toward the protein N-terminus.

Although class I HLA peptides are canonically characterized as 8-10mers, a substantial number of peptides were observed to belong to nested sets (7%; Table 3), suggesting the presence of a relatively high proportion of peptides binding in non-canonical conformations, such as bulge or overhang(29, 30). For example, if long isoforms of nested sets overhang, then the additional amino acids need not provide new anchors. On the other hand, if both short and long isoforms bind in tucked conformation, then extensions force the binding register to shift, and only certain amino acid additions can be tolerated. To investigate this further, the binding register of the peptide segment that binds to the HLA molecule was determined by comparing the predicted binding affinity of each peptide to that of the peptide sub-sequences within it (length 7 and greater). If at least one (sub)-sequence had predicted affinity of 500 nM or better and if that was 10-fold stronger than the runner-up (sub)-sequence, then the binding register was considered known (15% of peptides). Applicants observed that long isoforms indeed gain suitable new anchor sites (providing binding potential on par with the short isoforms); random amino acid extensions of short isoforms have uniformly worse binding potential (FIG. 9D). This suggests that most peptides bind in the canonical tucked conformation.

### Example 4

Evaluation of HLA-Peptide Characteristics that Impact HLA-Binding Predictions

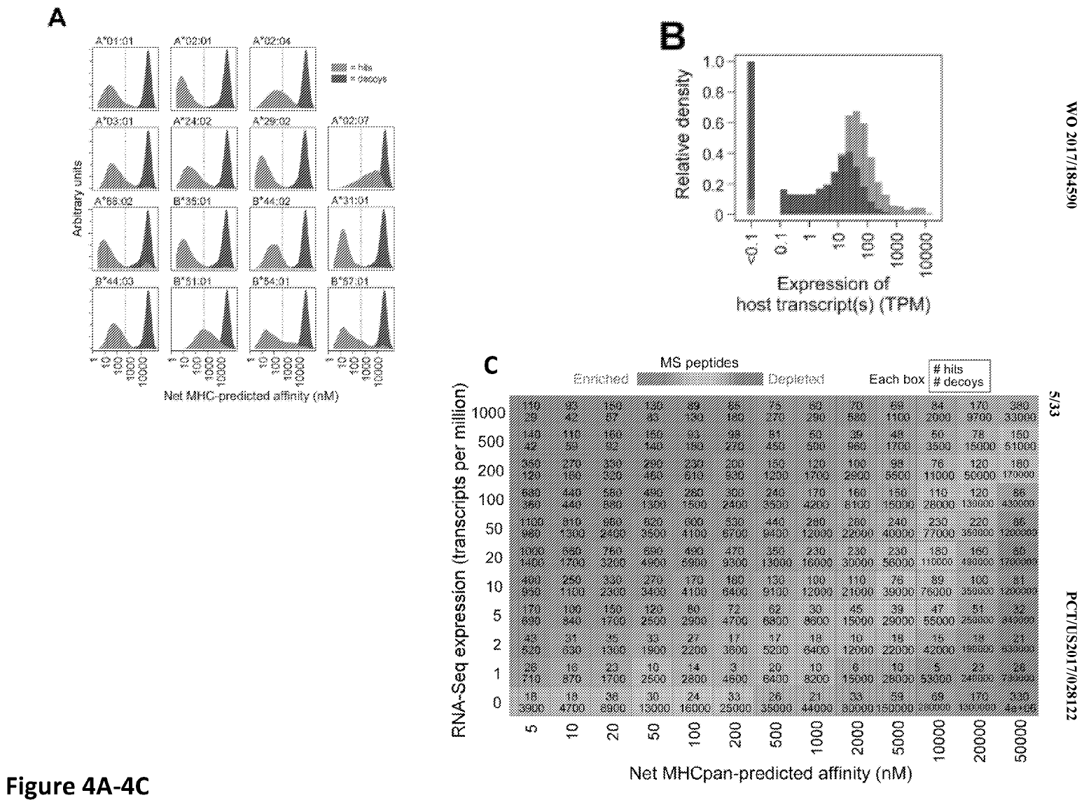

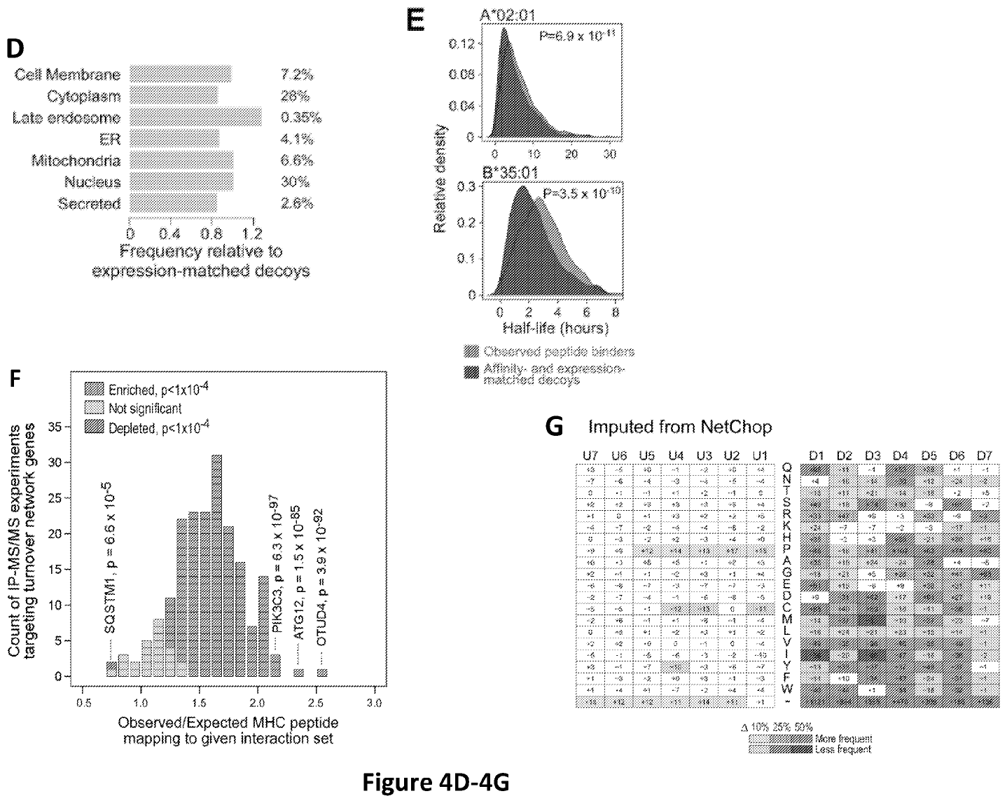

Applicants evaluated the extent to which various peptide characteristics were predictive of HLA-peptide presentation. The impact of HLA-binding affinity was first considered by comparing the distributions of NetMHCpan-2.8-predicted binding affinities of HLA-peptides sequenced by LC-MS/MS to those of 1×10⁶ random 9mer decoy peptides (FIG. 4A). For 8 of 16 HLA alleles, the distributions of peptide-binding affinities clearly separated from the random decoys at an IC₅₀ of ˜500 nM. Conversely, peptides identified from 3 alleles (HLA-A*02:04,-A*02:07,-B*54:01) demonstrated a distribution of weaker predicted binding affinities (>500 nM) that largely overlapped with random decoys. This result was likely due to insufficient existing IEDB data (only 90-661 peptide observations available per allele) that could be used for NetMHC training. The datasets from the remaining 5 alleles (A*03:01, B*57:01, A*68:02, B*35:01, B*51:01) revealed bimodally-distributed predicted affinities that overlapped in part with those of random decoy peptides. This observation suggested that the LC-MS/MS data captures new peptide-binding motifs not reflected in the IEDB.

Next, the impact of source protein expression was evaluated by comparing the expression distribution of all HLA-associated peptides sequenced by LC-MS/MS to that of 1×10⁶ random decoy peptides with varying transcription levels (FIG. 4B). By analysis of RNA-sequencing (RNA-seq) data from a representative single HLA transfected 721.221 cell line, Applicants observed that average expression levels of HLA-peptide source proteins were 10-fold higher than random source proteins (41.9 vs. 3.4 TPM), suggesting that highly expressed proteins are more likely to be processed and presented by the HLA class I pathway. To examine the relationship between expression and affinity, LC-MS/MS peptide observations across all alleles and the random 9mer decoys (1×10⁶ per allele) were binned according to these variables, and a peptide-to-decoy ratio for each bin was calculated in FIG. 4C. The likelihood of display was not strictly determined by affinity, but was rather a function of both gene expression and affinity. Highly presented peptides not only included peptides with strong binding affinity but also highly expressed peptides with weak predicted affinity. Conversely, lowly presented peptides included peptides with strong predicted affinity but low to absent expression. These data support the idea of expression-affinity ratio for improved peptide presentation prediction rather than use of simple affinity threshold cutoffs. This approach revealed a multiplicative relationship between expression and affinity, in which a 10-fold increase in expression could approximately compensate for a 90% decrease in binding potential. To rule out the possiblility that this finding might be an artifact of MS detection limits, Applicants compared the peptides with the highest versus lowest MS signal intensity and compared them in terms of RNA-Seq expression and predicted affinity. Low-intensity binders had lower expression and weaker affinity, showing that MS detection is not simply reflecting underlying protein abundance but also reflects relative binding strength (FIG. 9E). Though a simple kinetic model of peptide on- and off-rates may have predicted this limitations in expression data quality and depth and the use of multi-allelic data (for which prediction of affinity is more difficult) have previously obscured this finding. The presence of multiple upstream open reading frames in the 5′ UTR of a transcript is associated with reduced presentation potential for its associated peptides (FIG. 9F), suggesting that accurate measurements of translational efficiencies may enhance epitope selection further.

To determine whether HLA class I processing pathway has cellular localization biases, Applicants calculated the relative probability that a source protein from the LC-MS/MS dataset (pooled across alleles) was secreted or originated from the cell membrane, cytoplasm, late endosome, endoplasmic reticulum (ER), mitochondria, or cell nucleus compartments, relative to expression-matched, random 9mer decoys from protein coding genes (FIG. 4D, FIG. 9B). Without controlling for expression, the differences were dramatic, with secreted proteins showing an unexpected enrichment. However, the expression-corrected analysis eliminated most of these differences; no marked enrichment was observed in any particular cellular compartment, although peptides from the late endosome were 27% more frequent than in the decoy set. Peptides from secreted and ER proteins were modestly depleted—each about 15% less frequent than observed in the decoy set. Lack of expression correction may help explain why previous analyses of this question have reached inconsistent conclusions (Bassani-Sternberg et al., 2015, Mol. Cell. Proteomics 14, 658-673; Rock et al., 2014, Trends Immunol. 35, 144-152).

Studies of peptide presentation kinetics have suggested that specialized pathways exist that specifically target aborted translation products and misfoled proteins (Bourdetsky et al., 2014, Proc. Natl. Acad. Sci. 111, E1591-E1599; Yewdell, 2011, Trends Immunol. 32, 548-558). Consistent with recent analyses (Bourdetsky et al., 2014, Proc. Natl. Acad. Sci. 111, E1591-E1599; Kim et al., 2013, “Positional Bias of MHC Class I Restricted T-Cell Epitopes in Viral Antigens Is Likely due to a Bias in Conservation” PLoS Comput Biol 9, e1002884), Applicants did not see an enrichment of peptides at the N-termini of their source proteins (FIG. 3D), which would be expected if a meaningful fraction of peptides arose from aborted translation products. Applicants also considered whether peptides from proteins with a high instability index (Guruprasad et al., 1990, Protein Eng. 4, 155-161) or a high fraction of intrinsically disordered sequence were enriched in the MS data (FIGS. 9G and 9H) supposing that these would be more likely to trigger an unfolded protein response. The opposite trend was observed, suggesting either our measures of “foldability” were insufficient or that other unobserved variables potentially confound the signal.

Applicants considered whether pathways of normal protein turnover were tied to presentation likelihood. The count of ubiquitination sites (previously observed in KG-1, Jurkat, or MM1S cells (Kronke et al., Nature (2015) 523(7559); Kronke et al., 2014, Science (2014) 343(6168): 301-5; Udeshi et al., 2012, 2013, Molecular & Cellular Proteomics (2012) 11: 148-59), was positively associated with HLA-peptide presentation, consistent with the known role for ubiquitin in delivering proteins to the proteasome (FIG. 9I). Additionally, Applicants queried a collection of 200 IP-MS/MS experiments, each profiling the physical interaction partners of a protein involved in deubiquitination, autophagy, or ER-associated degradation (Behrends et al., 2010, Nature 466, 68-76; Christianson et al., 2012, Nat. Cell Biol. 14, 93-105; Sowa et al., 2009, Cell 138, 389-403) (FIG. 4H). Most of these gene sets were positively enriched in our data. Several outliers include P1K3C3, ATG12, and OTUD4, whose interation partners were most strongly enriched. Meanwhile, the interaction partners of the autophagosome cargo protein SQSTM1 were most depleted. Collectively, these analyses may help to point to turnover pathways with privileged access to the HLA presentation pathway.

Prior studies have identified the potential importance of peptide-binding stability on HLA-peptide presentation, which reflects a balance between both on- and off-rates, even after correcting for affinity (32, 33). The stability of peptides sequenced by LC-MS/MS was compared against affinity- and expression-matched decoys using NetMHCStab, a predictor trained on a large panel of HLA-peptide stability measurements for 10 highly expressed HLA alleles (33, 34). Of these alleles where NetMHCStab predictions were available, stability most dramatically affected HLA-B35:01 (FIG. 4E, FIG. 9C), with significant effects also observed for HLA-A*01:01 (p=1.2e-12), -A*02:01 (p=1.8e-15), -A*24:02 (p=1.1e-33), and -B*03:01 (p=3.1e-12) when using the Wilcox-rank sum test. Conversely, a negligible stability effect was detected for -A*03:01 (p=0.15). Notably, this result was not likely caused by insufficient training data for affinity prediction because none of these alleles have poor coverage in IEDB.

To calculate the relative contributions of variables like stability and affinity, various logistic regression models were developed and scored according to their positive predictive value (PPV) (see Methods). Applicants defined PPV as the fraction of LC-MS/MS peptides among the model's highest scoring peptides (top 0.1%) after all n MS-observed 9mer peptides were mixed with 999 n random 9mer decoys. A 0.1% threshold for positive calls was employed because this approximates the rate of true binders in a set of random peptides. Because there are approximately 10 million 9mers in the human proteome, of which each allele presents approximately 10,000, the 1:1000 ratio closely mimics the reality of the epitope selection problem. On the other hand, AUC (area under a ROC curve) calculations integrate performance over all possible thresholds. Thus, while AUC distributes weight of consideration across all thresholds (for example, calling >10% of peptides as positive; Table 4B),). The PPV approach appropriately focuses on performance among the most strongly positive calls, which is more consistent with epitope prioritization schema.

Each model included one or more of five predictor variables. Model performance was averaged across alleles with available stability prediction (FIGS. 5F and 5H, Table 4). Models based on affinity alone could achieve a PPV of 28-35% on average across 16 alleles (FIG. 5F; see Table 3 for individual allele results). A stability-only model (NetMHCpanStab (Jorgensen et al., 2014), model “S”) perfrmed nearly as well; however, joint prediction (model “AS”) showed minor synergism. Adding RNA-Seq or iBAQ-based (Ishihama et al., 2005, Mol. Cell. Proteomics 4, 1265-1272) protein expression (models “ASR” and “ASP”) improved PPV to 39% and 47%, respectively, while adding cleavage prediction (per a de novo predictor trained on other MS data) provided a 7.9% boost (prediction with NetChop yields 3.1%), and stability and localization provided only minimal improved performance (2% and 1%, respectively). Other putative processing variables (stability index, disordered sequence content, count of ubiquitin sites, and sequence features such as alpha helices and beta strands) likewise showed incremental improvements less than 1%. These data suggest that incorporation of gene expression information can improve prediction, while also suggesting that the greatest gains in prediction performance may still be driven primarily by refinement of sequence-based peptide-HLA affinity predictions.

By exhaustively testing all possible predictor combinations (Table 3), Applicants found the order of variable addition that added the most predictive value earliest and tracked the incremental PPV improvement provided by each variable, assigning this as the variable's “explanatory contribution” (FIG. 5H). Affinity and expression dominate the analysis, though notably, iBAQ-based protein expression provided negligible contribution beyond RNA-Seq. For the 45% of MS peptides that were missed in the full model, it was not known how much this related to the affinity and cleavage predictions being suboptimal, unknown variables, or stochasticity in the MS detection. The two genes with the most false negative calls per unit length were ubiquitin B and C, which suggests that improved understainding of protein turnover dynamics may be a key missing component.

### Example 5

Information from Peptide Sequencing by LC-MS/MS Improves Prediction of HLA-Peptide Binding

The binding affinity of peptides uniquely identified by MS but not well-represented within IEDB were experimentally measured to confirm the quality and predictive power of these data. Binary classification models (two single-layer artificial networks) were built for each of the 16 HLA alleles (see Methods) (FIG. 5G) and were used to select 33 peptides across five alleles (HLA-A*01:01,-A*29:02,-B*35:02,-B*51:01,-B*54:01) in which the predictive score for HLA presentation was in the top 10 percentile by MS-based models but bottom 10th percentile by NetMHCpan-2.8. These peptides tended to occupy regions on the 2D NMDS plots with fewer IEDB observations (FIG. 5A). By competitive peptide-binding assays, 32 of 33 peptides were confirmed to be strong binders (median IC50<14 nM). In contrast, only 12 of 33 and 13 of 33 were predicted to be binders by NetMHCpan-2.8 and NetMHC-4.0 respectively, based on a threshold of 500 nM (FIG. 5B, FIG. 10A).

Ensemble models of single layer artificial neural networks were developed by incorporating the following types of features: 3 sequence-encoding schemes (i.e. dummy, BLOSUM62, and fuzzy encoding (Methods, 18); amino acid properties (34); peptide characteristics (35); expression; and cleavage (Methods). Two types of ensemble models were trained, for which PPV and AUC were assessed: ‘MSIntrinsic’ which only utilized peptide-intrinsic features (sequence, amino acid properties, peptide characteristics), and ‘MSIntrinsicEC’ which additionally incorporated expression and cleavage information. To determine the number of peptides required to build a strong predictor, Applicants carried out saturation analysis by training models with varying number of positive training examples (minimum of 15 and maximum the full set of LC-MS/MS-identified peptides) and by measuring PPV on a test set of fixed size. Performance improvement was seen to level off at several hundred peptides (FIG. 5C). To understand why the machine-learned models performed better for some alleles, Applicants considered whether complexity of the peptide repertoire played a role. Indeed, a complexity score, defined as a decay-weighted average of the entropies at each peptide position, ranked the alleles with strongest performance, HLA-A*01:01,-B*44:03,-B*44:02,-A*29-02, as 1, 2, 3, and 5 of 16 respectively, from least to most complex (FIG. 5C).

For all alleles, the models trained on the LC-MS/MS data outperformed both NetMHC-4.0 and NetMHCpan-2.8 with an average PPV improvement of 20 and 30 percentage points for ‘MSIntrinsic’ and ‘MSIntrinsicEC’, respectively, in an internal 5-fold cross validation with 999 n decoys (FIG. 5D, Table 4A). Logo plots of decoys ranked within the top n positions suggest that binding motifs learned from the LC-MS/MS data are stricter than those learned from IEDB data (FIG. 10B). Conversely, NMDS visualization of false negatives suggests that they tend to be found at singleton or low-density clusters for MS-based models but also at high-density MS clusters for NetMHC models. (FIG. 10C). All algorithms scored similarly in terms of AUC, however, ‘MSIntrinsic’ and ‘MSIntrinsicEC’ demonstrated a significant improvement at very low false positive rate thresholds (FIG. 10D). Performance was also evaluated on 3 independent external data sources. First, a competition dataset of eluted 9mer peptides from the Dana-Farber Repository for Machine Learning in Immunology (DFRMLI) was considered. Data for both binders (average 335) and non-binders (average 1780) were available for HLA-A*02:02,-B*35:01,-B*44:03, and -B*57:01 (37). ‘MSIntrinsic’ performed better for 2 of 4 alleles compared to NetMHC-4.0 and NetMHCpan-2.8, even though this dataset had been incorporated into IEDB (Table 4B). Second, Applicants evaluated a curated set of 304 HIV-1 CTL Epitopes (38), which contained 52 9mer epitopes (that were shown to bound 12 of 16 HLA alleles in this study). Despite its limited size, this dataset provides a valuable opportunity for an evaluation which is orthogonal to MS-based peptide sequencing while remaining reflective of antigen processing and presentation rules. Binders for each allele were merged with all non-overlapping HIV 9mers and the rankings of epitopes provided in Table 4C. For 10 of 12 alleles, the top-ranked true epitope was at the same or higher position according to ‘MSIntrinsic’ as compared to NetMHC-4.0 or NetMHCpan-2.8. Finally, models were evaluated on an independent source of HLA class I LC-MS/MS data consisting of 7 cell lines expressing multiple HLA alleles (1). For each allele that overlapped with these data, binders of other alleles were heuristically excluded (i.e. any peptide with <150 nM affinity for another allele in the cell line and >1000 nM affinity for the allele being evaluated as predicted by NetMHCpan-2.8) and the remaining hits combined with 999 n decoys before PPV and AUC were calculated. Absolute PPV values were lower, due to incomplete allele deconvolution. However, consistent with internal evaluation results, the average PPV of ‘MSIntrinsic’ is 49% better than either NetMHC-4.0 or NetMHCpan-2.8, and the average PPV of ‘MSIntrinsicEC’ 97% better (FIG. 5E).

### Example 6

### Discussion

Applicants have in an unprecedented way enhanced the understanding of the rules governing antigen processing and presentation by developing a high-throughput workflow to rapidly characterize thousands of peptides naturally displayed on the surface of cell lines expressing single HLA alleles. Although LC-MS/MS-based approaches to identify the HLA-peptidome have long been employed, these studies have typically utilized primary cells or cell lines expressing the full complement of HLA molecules, making it challenging to distinguish allele-specific characteristics related to peptide display. With the single HLA allele-expressing cell lines as source material, together with refined experimental approaches and analysis strategies, Applicants could quickly generate a resource dataset of greater than 24,000 peptides associated with 16 class I HLA alleles. This ample dataset allowed Applicants to address anew the identification of allele-specific binding motifs, the factors impacting proteasomal cleavage, and the role of gene expression on peptide presentation. These insights were then translated into greatly improved prediction algorithms.

Although strong similarities among amino acid residues at anchor positions within HLA-peptides sequenced by LC-MS/MS (P2, P9) and existing IEDB peptides were detected, many novel anchors and sub-anchors were discovered. Across all alleles Applicants found that 11% of possible amino acid positions within 9mers were significantly different than those in IEDB, and a small set of peptides with distinct motifs were validated with a competitive binding assay. Although the analysis focused on 9mers, the present invention is applicable with 8mer and 10mer data, while at the same time, noting allele-specific differences. While most peptides fit the canonical model, exhibiting short length distribution (8-11 AA) and anchor residues in the second and last positions, Applicants observed peptides violating the length expectation (“bulge conformation” (29, 41, 42)) as well as an unexpected small population of peptides for which the anchors were not in the usual positions (39, 40). This was suggestive of possible overhang at both the N- and C-termini, a phenomenon more typically associated with Class II presentation. While more common among long peptides, this pattern was also evident in 9mer and 10mers. These observations invite further structural analysis as they could alter methods of antigen prediction that rely heavily on the identity of both N- and C-terminal anchor residues.

Conflicting proteomic studies have argued for and against correlations between protein abundance and HLA-peptide presentation (1, 43, 44). The present results, however, strongly support source protein expression as a highly predictive variable, with only HLA-binding affinity as a stronger driver of epitope prediction. Applicants evaluated the impact of expression through transcriptomic analysis using RNA-sequencing because this approach provides comprehensive and quantitative data for genes expressed at low levels that are difficult to measure using traditional proteomic methods. The observed correlation between the transcriptome and immunopeptidome supports the notion that antigens displayed by the HLA class I pathway represent the entire population of short-lived and stable cellular proteins that are processed by the proteasome, consistent with the “Proteome Model” for HLA class I peptide presentation (25). Notably, C-terminal cleavability was observed to provide only minor contribution, perhaps indicating proteasomes to have a more promiscuous specificity than previously reported in vitro studies. Likewise, cellular localization played a weak role in presentation, providing evidence that HLA class I-peptides are derived from endogenous proteins throughout the cell. Applicants also demonstrated that peptide-binding stability had a varied effect among the alleles tested. These differences may be an artifact of the data used for NetMHCstab training, or they may reflect biologically meaningful differences among the unique peptide-binding grooves of HLA alleles. Although multiple variables impacting HLA-peptide binding predictions were identified, it is difficult to know with certainty how much of the remaining prediction deficit should be attributed to insufficiency of the current predictor variable set vs. inherent stochasticity of the MS readout.

While similar artificial neural networks have already been successfully employed for peptide-MHC binding affinity predictions (7, 8), ‘MSIntrinsic’ and ‘MSIntrinsicEC’ performed better at identifying endogenously processed peptides. ‘MSIntrinsic’ appears to benefit from the unique nature of the LC-MS/MS dataset, which is more comprehensive and unbiased than IEDB for many alleles and is not subject to the same data heterogeneity. Meanwhile, ‘MSIntrinsicEC’ further benefits from the systematic incorporation of cleavability and expression information not available in IEDB. Recent therapeutic advances in cancer immunotherapy, such as those which activate T cells against tumor-specific epitopes, have showcased the promise of individualized epitope prediction as a therapeutic concept (45-49). The high quality and large size of MS-derived datasets stand to contribute significantly to the improvement of these prediction algorithms.

The present invention can expose other features, such as protein translation and degradation rates and peptide secondary structure that contribute to the unexplained portion of HLA-peptide predictions. Further improvements in database search FDR estimations can also improve the method. For instance, the calculation of motif-specific FDRs can enable more peptide identifications by rescuing some of the high quality peptide identifications that do not match dominant peptide-binding motifs. In addition, Applicants can expand the search strategy by including less common variable peptide modifications, accounting for germline and somatic protein sequence variations, and employing de novo search algorithms (50-52).

The methodologies described herein provide a path toward addressing new questions relating to HLA ligandomes. In particular, these workflows can be adapted to investigate the properties of HLA class II-binding peptides, for which a paucity of high quality data has severely limited prediction performance. In-depth analyses of the class II antigens could reveal novel immunotherapeutic targets because CD4+ T cell activation is crucial for eliciting vaccine-induced B cell and CD8+ T cell responses and may even be directly cytotoxic [Haabeth, Frontiers in Immunology, 2014; Haabeth, Leukemia, 2016]. Applicants can also apply the workflows to enable the sequencing of HLA class I and class II peptides presented by patient-derived cell lines and primary tumor samples, which can provide an opportunity to make the observations more direct and personalized. Overall, Applicants have developed novel technologies incorporating unbiased, direct HLA-associated peptide sequencing and downstream computational analyses that yield a comprehensive view of antigen processing and presentation that can advance all areas of immunology in which HLA-associated antigens are important, including cancer, infections, autoimmunity, allergy/asthma and transplantation.

### Material and Methods

HLA-peptide immuno-purification from 721.221 B cells and desalting. Single HLA class I allele-expressing B cells (13) were generated by transduction of the HLA class I negative 721.221 cells with a retroviral vector to express a single HLA class I allele as described previously (53) (cells expressing A*02:01, A*24:02 and B*44:03 purchased from the Fred Hutchinson Research Cell Bank, University of Washington; others gifted from Dr. E. L. Reinherz, DFCI). The class I HLA identities of the cell lines were confirmed by standard molecular typing (Brigham and Women's Hospital Tissue Typing Laboratory, Boston Mass.). Cells were cultured and HLA-peptide immuno-purification was performed as previously described (54, 55). Peptides were eluted from HLA complexes and desalted on in-house built Empore C18 StageTips (3M, 2315) (56). Sample loading, washes, and elution were performed on a tabletop centrifuge at a maximum speed of 1,500-3,000×g.

HLA-peptide immuno-purification of 721.221 B cells. Single HLA-allele expressing 721.221 cells were dissociated in lysis buffer in the presence of protease inhibitors and DNAse. Cells were subjected to sonication, and soluble lysates were collected after centrifugation and co-incubatd with Sepharose beads non-covalently linked to antibody. Beads were washed, dried, and stored until MS analysis. For example, 5-10×10⁷ single HLA-allele expressing 721.221 cells were dissociated using 2 ml of protein lysis buffer (20 mM Tris [pH 8.0], 1 mM EDTA, 100 mM NaCl, 1% Triton X-100, 60 mM n-octylglucoside, phenylmethylsulfonyl fluoride (Sigma-Aldrich, St. Louis, Mo.) and protease inhibitors (Complete Protease Inhibitor Cocktail tablets, Roche Life Science, Indianapolis, Ind.) 200 units of DNAse (Roche Life Science, Indianapolis, Ind.). This workflow was applied to 10 HLA-A expressing cell lines (A*01:01, A*02:01, A*02:03, A*02:04, A*02:07, A*03:01, A*24:02, A*29:02, A*31:01, A*68:02) and 6 HLA-B expressing cell lines (B*35:01, B*44:02, B*44:03, B*51:01, B*54:01, B*57:01). Cell membranes were further disrupted using 500 watts, 20kHz, QSonica500 sonicator (QSonica, Newtown, Conn.) at 35% amplitude using 10 sec pulses until all the visible precipitates were solubilized. Lysates were pre-cleared using microfuge centrifugation for 20 minutes at 12,000 rpm at 4oC. Soluble lysates were co-incubated with 20 μl of GammaBind Plus Sepharose beads (GE Lifesciences, Piscataway, N.J.) non-covalently linked to W6/32 antibody (Santa Cruz Biotechnology, Dallas, Tex.) for 3 hours. Beads were washed four times with lysis buffer without protease inhibitors, four times with 10 mM Tris (pH 8.0) and once with distilled water. Beads were dried and stored at −80° C. until MS analysis.

An example of HLA-peptide elution and desalting. StageTips were equilibrated with 2×100 μL washes of methanol, 2×50 μL washes of 50% acetonitrile/0.1% formic acid, and 2×100 μL washes of 1% formic acid. In a tube, the dried beads from HLA-associated peptide IPs were thawed at 4° C., reconstituted in 50 μL 3% ACN/5% formic acid, and loaded onto StageTips. The beads were washed with 50 μL 1% formic acid, and peptides were further eluted using two rounds of 5 minute incubations in 10% acetic acid. The combined wash and elution volumes were combined and loaded onto StageTips. The tubes containing the IP beads were washed again with 50 μL 1% formic acid, and this volume was also loaded onto StageTips. Peptides were washed twice on the StageTip with 100 μL 1% formic acid. Peptides were eluted using a step gradient of 20 μL 20% ACN/0.1% formic acid, 20 μL 40% ACN/0.1% formic acid, and 20 μL 60% ACN/0.1% formic acid. Step elutions were combined and dried to completion.

Whole proteome analysis of single-HLA allele expressing cell lines. 25 μg of trypsin-digested cell lysate (Mertins et al., 2013) from single HLA allele expressing cell lines, for example, HLA-A*29:02 and HLA-B*51:01 expressing cell lines, were fractionated using a previously described high-pH reverse phase StageTip protocol (Dimayacyac-Esleta et al., 2015). Five fractions were collected from each cell line using the following increasing acetonitrile concentrations (10%, 15%, 35%, 55%, and 80%), dried to completion, and reconstituted in 9 μL 3% acetonitrile/5% formic acid solution. Approximately half of each sample (4 μL) was analyzed in a single-shot MS run as described below. Greater than 70% overlap (>4,300 proteins) was observed between the unique protein identification (>2 unique peptides per protein) from HLA-A*29:02 (>5,200 proteins) and HLA-B*51:01 (>5,100 proteins) expressing cell lines.

HLA-Peptide sequencing by tandem mass spectrometry. All nanoLC-ESI-MS/MS analyses employed the same LC separation conditions described below. Samples were chromatographically separated using a Proxeon Easy NanoLC 1000 (Thermo Scientific, San Jose, Calif.) fitted with a PicoFrit (New Objective, Woburn, Mass.) 75 μm inner diameter capillary with a 10 um emitter was packed under pressure to ˜20 cm with of C18 Reprosil beads (1.9 μm particle size, 200 Å pore size, Dr. Maisch GmBH) and heated at 50° C. during separation. Samples were loaded in 3 uL 3% ACN/5% formic acid and peptides were eluted with a linear gradient from 7-30% of Buffer B (either 0.1% FA or 0.5% AcOH and 80% or 90% ACN) over 82 min, 30-90% Buffer B over 6 min and then held at 90% Buffer B for 15 min at 200 nL/min (Buffer A, either 0.1% FA or 0.5% AcOH and 3% ACN) to yield ˜13 (FA)-18 (AcOH) sec peak widths. During data-dependent acquisition, eluted peptides were introduced into either a Q-Exactive plus (QE+) or Q-Exactive HF (QE-HF) mass spectrometer (Thermo Scientific) equipped with a nanoelectrospray source (James A. Hill Instrument Services, Arlington, Mass.) at 2.15 kV. Resulting mass spectra were interpreted using the Spectrum Mill software package v5.1 pre-Release (Agilent Technologies, Santa Clara, Calif.). Instrument parameters and interpretation of LC-MS/MS data are described herein.

HLA-Peptide sequencing by tandem mass spectrometry. A full-scan MS was acquired at a resolution of 70,000 (QE+) or 60,000 (QE-HF) from 300 to 1,800 m/z (AGC target 1e6, 5 ms Max IT). Each full scan was followed by top 12 (QE+) or 15 (QE-HF) data-dependent MS2 scans at resolution 17,500 (QE+) or 15,000 (QE-HF), using an isolation width of 1.7 m/z with a 0.3 m/z offset, a collision energy of 25 (QE+) or 27 (QE-HF), an ACG Target of 5e4, and a max fill time of 120 ms (QE+) or 100 ms (QE-HF) Max ion time. An isolation offset of 0.3 m/z was used so that doubly charged precursor isotope distributions would be centered in the isolation window. HLA peptides tend to be short, <15 amino acids, so the monoisotopic peak is nearly always the tallest peak in the isotope cluster and the mass spectrometer acquisition software places the tallest isotopic peak in the center of the isolation window in the absence of a specified offset. Dynamic exclusion was enabled with a repeat count of 1 and an exclusion duration of 15 secs (QE+) or 10 secs (QE-HF). Charge state screening was enabled along with monoisotopic precursor selection using Peptide Match Preferred to prevent triggering of MS/MS on precursor ions with charge state 1 (only for alleles with basic anchor residues), >6, or unassigned.

Interpretation of LC-MS/MS Data. Mass spectra were interpreted using the Spectrum Mill software package v5.1 pre-Release (Agilent Technologies, Santa Clara, Calif.). MS/MS spectra were excluded from searching if they did not have a precursor MH+ in the range of 600-2000, had a precursor charge >5, or had a minimum of <5 detected peaks. Merging of similar spectra with the same precursor m/z acquired in the same chromatographic peak was disabled. MS/MS spectra were searched against a database that contained all UCSC Genome Browser genes with hg19 annotation of the genome and its protein coding transcripts (63,691 entries; 10,917,867 unique 9mer peptides). A two-round search strategy was used. Prior to both search rounds, all MS/MS had to pass the spectral quality filter with a sequence tag length >2, i.e. minimum of 3 masses separated by the in-chain mass of an amino acid. In the first round search, all spectra were searched using a no-enzyme specificity, fixed modification of cysteine as unmodified, no variable modifications, a precursor mass tolerance of ±10 ppm, product mass tolerance of ±20 ppm, and a minimum matched peak intensity of 50%. Peptide spectrum matches (PSMs) for individual spectra were automatically designated as confidently assigned using the Spectrum Mill autovalidation module to apply target-decoy based FDR estimation at the PSM level to set scoring threshold criteria. Peptide autovalidation was done separately for each HLA allele with an auto thresholds strategy using a minimum sequence length of 7, automatic variable range precursor mass filtering, and score and delta Rank1-Rank2 score thresholds optimized across all LC-MS/MS runs for an HLA allele. This yielded a PSM level FDR estimate for precursor charges 1 thru 4 of <1.0% for each precursor charge state. All confidently identified peptides for each allele used to define HLA-specific cleavage specificity. In the second round search, all remaining spectra that that were not confidently identified in the first round were searched using the HLA-specific cleavage specificity with the following allowed variable modifications added: oxidized methionine, pyroglutamic acid (N-term q), deamidation (n), cysteinylation (c), and phosphorylation (s,t,y). An additional round of FDR thresholding as described above was applied to PSM's from the second round search. The combined PSM's from each round had a peptide level FDR <2.0% for each HLA allele.

The creation of decoy sequences during the Spectrum Mill search was adapted so that the target decoy thresholding above better mimicked HLA-peptide populations. Decoy sequence generation typically involves reversing an entire protein sequence (preserves enzyme cleavage frequency), scrambling peptide sequences randomly, or reversing the internal sequence while keeping the ends fixed to enable FDR estimation within a specified confidence interval based on the levels of decoy and target matches (58). When generating decoys in Spectrum Mill for every sequence passing the precursor mass filter the peptide C-terminus was held fixed during the no enzyme search round. The second position was additionally held fixed during the HLA allele-specific cleavage round since HLA-associated peptides contain anchor residues at position 2 and last position.

Sequence properties of MS-identified peptides compared to IEDB. A curated set of previously identified class I HLA-bound peptides was downloaded from the Immune Epitope Database (IEDB) at http://www.iedb.org/ (accessed on Oct. 26, 2015) (Vita et al., 2015). For each allele, IEDB peptides with a measured affinity <500 nM were compared to MS peptides in terms of their length and positional amino acid frequencies. In addition, a metric was defined for the pairwise “distance” between 9mers (a Hamming distance calculated using an amino acid substitution matrix (Kim et al., 2009, BMC Bioinformatics 10, 1-11) and inversely weighted according to positional entropy) and used to cluster MS and IEDB peptides in a 2-dimensional representation. A machine learning approach identified peptides with motifs favored in the MS but poor-scoring according to NetMHCpan-2.8; the MHC-binding affinities for these peptides were determined by competitive binding per gel filtration protocol (Sidney et al., Current Protocols in Immunology, (John Wiley & Sons, Inc., 2001).

Quantifying contributions to peptide presentation potential. To quantify the relative contribution of each explanatory variable, logistic models were built to discriminate hits from random genomic 9mer decoys. Several of these variables had highly non-normal distributions and were transformed. Predictive performance for a given variable set was quantified using a logistic model fit to differentiate all observed 9mers for a given allele from 999 n random genomic 9mers. The 0.1% top-scoring peptides were considered as positives, and positive predictive value (TP/(TP+FP)=PPV) was assigned according to this threshold. The average PPV score per model was calculated across all alleles with available stability prediction (HLA-A01:01, HLA-A02:01, HLA-A03:01, HLA-A24:02, and HLA-B35:01). Variables were progressively added to the model and the increase in PPV at each step was used to assess the incremental contribution of each variable. The order of inclusion was determined by each variable's solo predictive power, except for stability, which was included last.

Peptide-Binding Assay. A subset of peptides were synthesized (RS Synthesis, Louisville Ky.) and tested for binding to HLA molecules (IC₅₀<500 nM) by competitive MHC class I allele-binding per gel filtration protocol (57).

Machine learning. HLA-peptides sequenced by mass spectrometry along with a set of random decoys were used to build binary classifiers (one classifier per HLA allele) to predict whether a given peptide will bind to a specific HLA allele. Generalized linear models were first trained with the glmnet R package in a 5-fold cross-validation scheme. Theano was used to train two types of neural networks: three models which incorporate one of the sequence encoding schemes with the rest of the peptide-intrinsic features (amino acid properties, and peptide characteristics), and three models which incorporate one of the sequence encoding schemes with all other features (including expression and cleavage). Scores of three models in each group were averaged together in an ensemble.

Database Search Evaluations. The validation yield (number of valid PSMs/filtered PSMs) across our HLA datasets was calculated to be approximately 9% (range of 2%-26%). This median validation yield was similar to the identification rate reported for high-energy collisional dissociation (HCD) only HLA-associated peptide sequencing (48). Applicants then compared our HLA-A*02:01 allele dataset to a high resolution dataset recently published for the HLA-A*02:01 positive B cell line, JY (1) (FIG. 6B). Both datasets were searched using our strict filtering criteria and no enzyme specificity, as this was the specificity used by Bassani-Sternberg et al. A large degree of unique peptide overlap between our biologic replicates (71%) was observed, while a lower overlap (42%) was observed between two biological replicates of JY reported. Applicants also calculated the number of PSMs that passed our strict quality filters and 1% FDR estimation cutoff from the no enzyme and HLA-specific rounds of database searching (FIG. 6A).

Assessment for MS bias. To assess whether data gathered via mass spectrometry may exhibit technical biases, Applicants first utilized the Enhance Signature Peptide (ESP) algorithm (Fusaro et al., 2009, Nature Biotechnology (2009) 27, 190-198) to predict high-intensity peptides (“MS Observability Index”) within peptides in the MS dataset as well as within peptides recorded in the IEDB. Fourteen out of the 16 alleles in the study were included in this analysis due to the very low number of peptides in IEDB for two of the alleles: HLA-A*02:04 and HLA-A*02. Since anchor positions have allele-specific residue preferences and more data is available for some alleles than others, Applicants considered 300 9mer binders chosen at ramdom for eah of the 14 alleles from each dataset (MS and IEDB), where for alleles with less than 300 identified binders the random sampling was performed with replacement. With the data thus formed, the ESPPredictor (available on GenePattern http://genepattern.broadinstitute.org/) was run for each peptide and the distributions of observability scores of peptides in the two data sets were compared (FIG. 12A). To further proble for technical biases, Applicants used the same data to evaluate the frequency of occurrence of each of the 20 amino acids within peptides in the MS and peptides in IEDB (FIG. 12B). Similarly, amino acid frequency was also compared between peptides in IEDB and two additional external mass spectrometry data sets (Bassani-Sternberg et al., 2015, Mol. Cell. Proteomics 14, 658-673; Trolle et al., 2016, J. Immunol. (2016), doi:10.4049/jimmunol.1501721) (FIG. 12F).

Sequence Properties of MS-Identified Peptides Compared to IEDB

### IEDB Dataset

A curated set of previously identified HLA-I bound peptides was downloaded from the Immune Epitope Database (IEDB) at http://www.iedb.org/ (accessed on Oct. 26, 2015) (Vita et al., 2015). The ‘MHC Assay Details’ option under ‘Specialized Searches’ was used and all ‘Linear Epitopes’ (under ‘Epitope’ menu box) associated with ‘MHC Class I’ (under ‘Assay’ menu box) were selected for each of the 16 alleles in our study. Furthermore, any epitope, which did not have a quantitative measure, was excluded.

### Affinity and Length

For each allele,MS-observed 9mer peptides were scored by NetMHCpan-v2.8 and compared to 1 million random 9mers drawn from the proteome (FIG. 4A). MS peptides (all lengths) were assessed in terms of their length distributions (FIG. 1C).

### Heatmap of Positional Amino Acid Differences

Applicants tabulated the amino acid counts for each allele at each position (1-9) within 9mer peptides, first for the MS dataset and separately for the IEDB dataset (for IEDB data, peptides with measured binding affinity of less than 500 nM were considered). Alleles HLA-A*02:04 and HLA-A*02:07 have less than 10 binders peptides in IEDB and were excluded from the analysis, leaving 14 out of the 16 alleles in our study. At each (allele, position, amino acid) tuple, the number of peptides which contain the amino acid and the number of peptides which do not are counted and a chi-squared test is used to asses for differences between the MS and IEDB data sets.

### Sequence Logo Plots

To capture and compare binding motifs between groups of peptides, sequence logo plots were generated using the motifStack R package (FIG. 7A).

### Entropy

The entropy at each 9mer position (1 through 9) was calculated for each allele based on all LC-MS/MS 9mer peptides identified for that allele (MS entropy) and then similarly for all IEDB 9mers binders (nM<500) (IEDB entropy). The computation was performed with MolecularEntropy( ) function from HDMD R package, where entropy values are normalized by log(20) such that entropy of 0 indicates a position with no variation while entropy of 1 indicates that all amino acids are equally likely to be observed at that position (FIGS. 2B & 7B).

### Peptide Distance

The following peptide distance metric was defined and computed between every pair of 9mer peptides in the MS and IEDB sets:

$$d ( {s}_{1} , {s}_{2} ) = \frac{1}{9} {\sum }_{i = 1}^{9} distPMBEC ( {s}_{1 i} , {s}_{2 i} ) * ( 1 - {entrophy}_{i} ) ,$$

where s₁ and s₂ are peptide sequences (e.g. KVLPIIQRW and HSRPIVTVW); sig is the amino acid at position I of the first peptide sequence; PMBEC is a pre-calculated matrix of residue similarities biased by their HLA binding properties (Kim et al., 2009) and distPMbEC, defined as max(PMBEC)-PMBEC, is a 20×20 matrix capturing residue dissimilaries; entropy is the [0,1]-scaled entropy at position i for the allele associated with s₁ and s₂. The average of MS and IEDB entropy was used in the distance metric computation.

### Peptide Distance Visualization and Clustering

A pairwise peptide distance matrix was computed between every pair of peptides 9mer peptides in the MS and IEDB sets as described above. Since the matrix contains relative peptide distances rather than absolute Cartesian coordinates, Applicants used non-metric multidimensional scaling (NMDS) to visualize the peptides in two demotions (nmds( )function from ecodist R package). Density based clustering was then performed to assign peptides to clusters with dbscan( ) function from package dbscan (FIG. 8).

### Further Assessing for Mass Spectrometry Bias

To assess the possibility that MS data clusters closely together due to mass spectrometry-related technological biases, Applicants considered only the subset of peptides from MS and IEDB datasets with physicochemical properties that are favorable for MS detection. Namely, Applicants selected peptides with one charged residue (by counting the R, H, and K residues per peptide) and peptides with moderate hydrophobicity by removing peptides which had hydrophobicity scores in the lowest and highest decile (a hydrophobicity score for each peptide was assigned with the hydrophobicity( )function in Peptides R package). Analysis of the average peptide distances between MS and IEDB datasets and NMDS visualizations per allele were then carried out for this subset of favorable for MS peptides (36% of IEDB and 54% of MS peptides remained; alleles HLA-A*02:04 and A*02:07 were excluded due to low number of IEDB peptides), where the number of peptides was samples to be equal in the two data sets (FIGS. 2E & 2A).

### Direct Affinity Measurement

To determine whether the MS dataset can be used to predict novel HLA-bound peptides, Applicants built a binary (bound/not bound) generalized linear model for each of the 16 only using the MS data in addition to a random set of decoys from the proteome. Applicants used these models to score each MS peptide. MS peptides were also evaluated with NetMHCpan-2.8 and those that scored in the top 10 percentile by MS-based models but bottom 10th percentile by NetMHCpan-2.8 were selected for experimental validation. Thirty tree peptides across five alleles (HLA-A*01:01,-A*29:02,-B*35:02,-B*51:01,-B*54:01) were synthesized (RS Synthesis, Louisville Ky.) and tested for binding to HLA molecules (IC50<500 nM) by competitive HLA class I allele-binding per gel filtration protocol (Sidney et al., 2001) (FIGS. 5A and 10A).

Peptide Processing Analyses. For each MS hit, the upstream 10 amino acids and downstream 10 amino acids were determined. To account for peptides near the beginning or end of their source protein, a 21st “amino acid”, denoted as “-”, was introduced to represent blank positions. For the minority of hits mapping to multiple genes, a selection was made at random. Each MS peptide was matched to 100 random 9mer peptides (drawn from the human proteome) but matched according to the first two and last two amino acids (to control for confounding signals from non-random sequence patterns in the proteome). In comparing the sequence context of theMS hits to the sequence context of the decoys (FIG. 3A), the relative enrichment for each amino acid at each position was calculated as a percent change, and the significance was calculated by chi-squared 2×2 contingency table test. Additional previously published MS datasets representing other cell types were analyzed using this same approach (FIGS. 11B-G). The amino acids frequency analysis in FIG. 12C, which considers amino acids frequencies within the peptide, uses a separate set of decoys comprising 1,000,000 9mers drawn at random from the proteome (i.e. no matching on first two and last two amino acids of the peptide).

To understand the motifs favored by the cleavage prediction algorithm NetChop (Keşmir et al., 2002, Protein Eng. 15, 287-296; Nielsen et al., 2005, Immunogenetics 57, 33-41)

(FIG. 4I), 1,000,000 random proteome 9mers and their corresponding sequence contexts were scored by the algorithm. The top-scoring 25% and bottom-scoring 25% were identified and analyzed in the manner of FIG. 3A (top 25% treated as hits; bottom 25% treated as decoys).

To assess whether peptides might be enriched or depleted with respect to source protein sequence features, every MS peptide was matched to ten random 9mers from the same source gene. Then each hit or decoy was marked according to whether it intersected one of the Uniprot (ftp://ftp.uniprot.org/pub/databases/uniprot/knowledgebase/uniprot_sprot.dat.gz) sequence features: “STRAND”, “HELIX”, “TURN”, “SIGNAL”, or “COILED”. The relative frequency of these features was calculated for hits and decoys, and p-values were calculated by chi-square test (FIG. 12D).

To determine how the relative expression of proteasome and immunoproteasome components in B721.221 cells compared to other tissues (FIG. 12D), expression values (represented in transcripts per million) were compared against high-purity TCGA tumors (>95% according to the “percent tumor cell” field in the clinical slide review). If more than five samples were of sufficient purity for a given tumor type, only the top five were used.

To determine whether peptides were likely to be binding in non-canonical overhang conformations, 9mer and 10mer pairs were identified where the sequences were identical aside from 1 additional amino acid at the 10mer's n- or c-terminus (i.e. an “extension” of the 9mer, which one might presume binds with overhang). For each pair, another 100 10mers were simulated by extending the 9mer with a random amino acid (sampling at proteome frequencies). This procedure was repeated with 9mer+11mer pairs, and three peptide groups—the “core” 9mers, the “extended” 10mers and 11mers, and the simulated 10mers and 11mers—were compared in terms of their predicted binding affinities. Binding predictions were made by concatenating the first 5 and last 4 amino acids of each peptide and processing it with NetMHCpan-v2.8 as a 9mer. This prediction approach assumed that anchors remain at a fixed distance from the peptide termini (regardless of peptide length), which should be true if peptides always bind in a “tucked” conformation rather than an “overhang” conformation. If overhang conformation was common among the 10mers and 11mers of these nested sets, then the true 10mers and 11mers would not be expected to have better binding scores than the simulated 10mers and 11mers. On the other hand, if the true 10mers/llmers have similar scores as the 9mers, it suggests that nested sets only occur when short and long isoforms can both achieve a tucked conformation (strongly suggesting the overhang occurs rarely or never; (FIG. 9D).

Quantifying Variables Associated with HLA Presentation

### Relationship Between Expression and Affinity

RNA was isolated from B721.221 cells expressing a single HLA allele, for example, HLA-A*29:02-, B*51:01-, B*54:01-, and B*57:01 (RNeasy mini kit, QIAGEN), processed to cDNA (e.g., Nextera XT kit; Smart-seq2 protocol), sequenced (e.g., HiSeq2500, Rapid Run mode; 50bp paired-end), and aligned (e.g., bowtie2-2.2.1 (Langmead and Salzberg, 2012); UCSC hg19 annotation). Transcript expression (RSEM-1.2.19 (Li and Dewey, 2011); GEO accession GSE93315) were averaged across the 4 cell lines and adjusted by dropping non-coding transcripts and rescaling TPM values to sum to one million. Expression of each peptide source protein was determined by summing all transcripts containing the peptide.

To assess the relationship between expression and affinity, the 9mer MS peptides for each of the 16 profiled alleles were binned according to their predicted expression and affinity (NetMHCpan-v2.8 prediction). Meanwhile, 1,000,000 random proteome decoy 9mers were binned in the same manner (for each allele). Finally, for each expression-affinity bin, the ratio of MS hits to decoys was calculated (FIG. 4C).

To understand the potential differences between observed MS peptides and HLA ligands that fail to be sampled in the MS, Applicants identified peptides that were readily detected (top 10% of precursor ion intensity) to those that were just barely detected (bottom 10% of precursor ion intensity). Expression and affinity values (per NetMHCpan-v2.8 prediction) were compared for the two peptide sets (FIG. 9E).

To identify the potential impact of translational efficiency, the count of ATG 3mers upstream of the canonical ATG start codon was determined for each protein coding gene (per UCSC annotation). EachMS hit was matched to 10 9mer decoy peptides, which were chosen based on having similar RNA-Seq expression (minimum absolute log fold change) but different source gene. To avoid having all 10 decoys come from the same gene (which would add noise to the analysis), they were required to come from 10 different genes. In this manner, hits could be compared to decoys in terms of the relative count of upstream ATGs in a manner controlled for relative gene expression (FIG. 11C). The significance of the association was determined by t-test (comparing the upstream ATG counts of hits vs. decoys).

### Impact of Processing Pathways

MS peptides were compared to decoys (10 decoys per MS peptide; each from a different gene; matched per transcript expression) in terms of various features potentially related to peptide processing: UNIPROT localization (www.uniprot.org), distance from protein N-terminus, source protein stability index (Guruprasad et al., 1990, Protein Eng. 4, 155-161), intrinsically disordered sequence content (http://d2p2.pro) (Oates et al., 2013, Nucleic Acids Res. 41, D508-D516), count of known uniquitination sites (Eichmann et al., 2014, Tissue Antigens 84; Kronke et al., 2015, Nature 523(7559); Udeshi et al., 2012, Molecular & Cellular Proteomics (2012) 11: 148-59), and physical interaction with known protein turnover regulators (Behrends et al., 2010, Nature 466, 68-76).

### Assessing for Aborted Translation

Two vectors (length 30000) representing protein positions originating at the N-terminus (initialized to zeros) designated O (“observed”) and E (“expected”) were created. For each hit, 1 was added to the position determined for each peptide within the host transcript, and 1/n was added to positions 1 through n in E, where n is the total number of positions that the peptide possibly could have come from (the total length of the protein minus the length of the peptide). The resulting O/E ratio, representing the ratio of observed to expected hits per position, were binned setting the bin length to 100 each.

Assessing Bulge vs. Overhang Conformation

For each hit peptide, the affinity for each constituent sub-peptide of length 7 or greater was scored. To estimate affinity for a peptide of arbitrary length, the first 5 amino acids and the last 4 amino acids were concatenated and scored with NetMHCpan-2.8. The binding register of a hit peptide was considered a confident identification if the best sub-peptide had predicted affinity less than 500 nM and was at least 10× stronger than the second best sub-peptide. For the peptides for which the best sub-peptide was shorter than the full-length peptide, Applicants considered the position of the sub-peptide within the host peptide and the count of extra residues on the C-terminal and N-terminal side and these results were tabulated.

### Affinity

Affinity for each sequenced 9mer for the HLA molecule it was eluted from was estimated using NetMHCpan-v2.8(7). Expression levels of peptides were determined using RNA-Seq data from four libraries (prepared from the A29:02-, B51:01-, B54:01-, and B57:01-transfected cell lines) that were aligned to the UCSC transcriptome annotation (downloaded June 2015) using Bowtie2 (bowtie2-2.2.1, default parameters (59)). Gene expression was quantified according to RSEM (rsem-1.2.19, default parameters (60)). Records for non-coding transcripts (per the UCSC annotation) were dropped and transcript per million (TPM) values were re-scaled and averaged across the four cell lines to yield a single expression value for each protein-coding transcript. The expression level of a peptide (hit or decoy) was determined as the sum of the expression levels of the transcripts containing that peptide. Expression and affinity bins were also defined for each allele by counting the number hits and decoys in each bin, and a binder:decoy ratio per bin was calculated by merging this analysis across alleles.

### Localization

Localization information was obtained from “SUBCELLULAR LOCATION” records in Uniprot's curated protein annotation (ftp.uniprot.org/pub/databases/uniprot/current_release/knowledgebase/complete/uniprot_sprot.dat.gz). Uniprot's ID mapping table (ftp.uniprot.org/pub/databases/uniprot/current_release/knowledgebase/idmapping/by_organism/HUMAN_9606_idmapping.dat.gz) as well as the UCSC-to-Uniprot ID mapping available from UCSC table browser (https://genome.ucsc.edu/cgi-bin/hgTables) were used to sync these data with UCSC annotations. Proteins were tagged as “Cell Membrane” if the localization field contained the text “cell membrane”; “Mitochondria” if “mitochondr”; “Nucleus” if “nucle”; “Cytoplasm” if “cytoplasm”; “ER” if “Endoplasmic reticulum”; “Secreted” if “secret”; “Late Endosome” if “late endo”. It was possible for a protein to be associated with more than one localization. A set of decoy peptides was constructed by matching each hit peptide to a decoy with similar expression because different cellular compartments tend to be expressed at different levels.

### Stability

Stability predictions for hit peptides were generated using the NetMHCStab algorithm (33) for alleles available at time of publication: HLA-A01:01, HLA-A02:01, HLA-A03:01, HLA-A24:02, and HLA-B35:01. Because NetMHCStab has limited maximal throughput, stability predictions could not be calculated for the large set of 1e6 decoys. Rather, each hit peptide was matched to a single decoy with the most similar predicted affinity. Density plots were created to compare the hits for each allele against the corresponding affinity-matched decoys.

### Unfolded Protein Response

All proteins in the proteome were scored according to a sequence-based estimate of protein instability(Guruprasad et al., 1990). MS hits and expression-matched decoys (using the expression-matching approach employed in FIG. 9F) were binned according to the instability scores of their source proteins. The relative ratio of hits in each bin was compared to that observed for the decoys (FIG. 9G). The significance of the association was determined by ttest (comparing the instability scores of hits vs. decoys).

In a second analysis, all protein-coding genes were assessed in terms of their content of intrinsically disordered sequence. Disordered sequence predictions from 6 tools (iupred-1.disrange, iupred-s.disrange, espritz-d.disrange, espritz-n.disrange, espritz-x.disrange, and anchor.disrange; http://d2p2.pro(Oates et al., 2013) were available for the Gencode V19 human gene annotation; 12mers that were disordered according to three or more of the predictors were identified and counted for each gene in the UCSC gene annotation. MS hits and expressionmatched decoys were compared according to the percent disorder (disordered 12mers divided by total 12mers) in their source proteins (FIG. 9H). The significance of the association was determined by t-test (comparing the percent disorder of hits vs. decoys).

### Ubiquitination

Previously published ubiquitin-targeting IP-MS/MS experiments in KG-1, Jurkat, or MM1S cells (Kronke et al., 2015; Krönke et al., 2014; Udeshi et al., 2012) were pooled to define a set of putative ubiquitination sites, and these sites were counted per gene in the UCSC annotation. Hits and expression-matched decoys were compared in terms of their counts of ubiquitination sites, and significance was determined by t-test (comparing the site counts in hits vs. decoys). The p-value is presented as “0” since it was less than the machine precision of our operating system (approximately 1×10⁻³⁰⁰) (FIG. 9I).

### Protein Turnover Pathways

Results from nearly 200 IP-MS/MS experiments targeting various protein turnover pathways genes (http://besra.hms.harvard.edu/ipmsmsdbs/cgi-bin/downloads.cgi;http://www.nature.com/nature/journal/v466/n7302/full/nature09204.html) were downloaded, and the protein identifications in each experiment were sorted according to their “Weighted D-Score”, a measure of confidence that the given protein physically interacts with the bait. Each set was trimmed to include the only top 100 identifications to deplete it of non-specific binders. Then, for each set, Applicants counted the number ofMS hit peptides (vs. the number of expressionmatched decoy peptides) that could be assigned to a protein in the set. Enrichment was assessed as the rate of hits in the set divided by the rate of decoys in the set, and the p-value was determined using a chi-squared 2×2 contingency table (FIG. 4H).

### Peptide Cleavage

Upstream cleavage of peptides observed across all alleles were systematically compared against random decoy peptides with the same first three amino acids (“3mer-matched”). The 3-mer matching approach accounted for non-random sequence patterns in the genome that might otherwise confound the analysis. The frequency of amino acids in upstream positions were determined for each hit and corresponding decoys. The relative enrichment for amino acid was calculated as a percent change while significance was calculated by paired t-test. Blank positions resulting from peptides from the N-terminus of proteins were considered as “-”. An analogous procedure was followed where hits were matched to random decoys with the final 3 amino acids to analyze downstream enrichments. A simple logistic model was built to estimate C- and N-terminal cleavability that discriminated decoys from hits (10:1). Multi-mapping peptides were assumed to arise from the transcript with the highest expression. This analysis did not consider enrichments internal to the presented peptide because these would be confounded by the HLA peptide-binding motifs unique to each allele. A predictor that assessed the overall cleavability of a peptide in the same manner as NetChop (23,24) was also built using a logistic regression in which each input variable was the N-terminal or C-terminal cleavability.

### Transformation of Variables Prior to PPV Calculations

1. The log of the hit:decoy ratio was calculated for different affinity bins and the overall curve was smoothed using the isoreg( ) function in R (61). This log-ratio value was used rather than nM affinity directly.
2. Likewise, for expression, the log of the hit:decoy ratio was calculated for different expression bins and the overall curve was smoothed using isoreg( ). This log ratio was used rather the TPM expression directly.
3. Seven dummy (0/1) variables were created to encode the various possible cellular localizations.
4. NetChop and MS-based cleavage probabilities were converted to log odds (log(p/(1−p)))
5. Stability predictions used half-lives.

### Machine Learning Model Features

Five different classes of features were used for machine learning in various combinations:
1. Peptide sequence—180 features. Each 9mer peptide amino acid sequence was represented as a numerical vector of length 180 in three ways 1.1) dummy (or binary) encoding, 1.2) blosum62-based encoding, 1.3) a fuzzy encoding where the each position in the vector represent the similarity between the true amino acid at the current peptide position with each of the 20 amino acids according to the PMBEC matrix (19).
2. Amino acid properties—27 features. Each residue in a peptide was represented by the first three principle components of PCA on amino acid properties (27 features) (35).
3. Peptide properties—8 features. The following peptide characteristics extracted from the

Peptides package in R were used: “boman”, “hmoment”, “hydrophobicity”, “helixbend”, “sidechain”, “xstr”, “partspec”, “pkc”.
4. Expression—1 feature. log₂(TPM+1) expression (as measured here);
5. Cleavability—1 feature. MS-based cleavage score (as defined above).

### Linear Models

Linear models that only included the 180 dummy coding features were trained with glmnet R package. At the end of 5-fold training, the test results from each fold were assembled into the full data set and used to compute the area under curve (AUC) and PPV (as defined in main text). This was repeated with three different random initializations of the fitting procedure. The full training data set included all 9mer LC-MS/MS peptides identified as well as a set of 10× random decoys. Performance of the model was compared to NetMHCpan-2.8 on the same set of hits and decoys.

### Neural Network Models

Artificial neural networks were built following the same cross-validation procedure with an equal number of positive and negative training examples: a random sample of all hit peptides of size 10× the number of hits was taken (with replacement) and supplemented with a random set of decoys of the same size. The network architecture for the ‘MSIntrinsic’ model consisted of an input layer with 215 features (peptide sequence 180+amino acid properties 27+peptide properties 8) and single hidden layer with 50 hidden units. The final model scores were defined as the average of the outputs of 3 networks trained with different random initialization seeds. To compose the ‘MSIntrinsicEC’ ensemble model, first neural networks with 182 features (peptide sequence 180+expression 1+cleavability 1) and the same number of hidden layer units were trained with 3 random initilizations. The Final ‘MSIntrinsicEC’ scores were then calculated by taking the average of these networks and the ‘MSIntrinsic’ networks. The same 5-fold splits were used to train both types of neural networks to ensure ‘MSIntrinsicEC’ improvements were not due to seeing more positive training examples. All neural network training was done using Theano and code development followed the deep learning tutorial at deeplearning.net/software/theano/.

### Development of New Epitope Selection Algorithms

For each allele, neural network classifiers (one hidden layer with 50 units) were trained (using Theano (Theano Development Team, 2016); 5-fold cross-validation) to differentiate MS 9mers from random decoy 9mers using different input feature schemes: dummy encoding, BLOSUM62, PMBEC (Kim et al., 2009), biochemical properties (Bremel and Homan, 2010), and peptide-level features (D. Osorio, P. Rondon-Villarreal, R. Tones, 2014); the results of these models were averaged to obtain a single prediction (called MSIntrinsic). A second prediction (MSIntrinsicEC) was made by adding expression and MS-trained cleavability. Performance was validated on external data by measuring PPV (fraction of true MS peptides among the top-scoring 0.1%, where decoys are present at 999:1). For multi-allelic data sets, the evaluation excluded any MS peptides that obviously belonged to an HLA- or HLA-B allele other than the one in question (e.g. if predicting for A01:01 for a cell line with genotype A01:01, A02:01, B35:01, B44:02, MS-observed peptides with NetMHCpan-2.8 scores worse than 1000 nM for A01:01 and better than 150 nM for A02:01, B35:01, or B44:02 were excluded).

To determine the synergism that might be achieved with models that incorporate multiple variables (predicted affinity, expression, cleavability, etc.), Applicants built various logistic regression models (for each allele) to discriminate n MS-observed peptides from 999 n decoy peptides. Since some predictor variables had highly non-normal distributions, they were transformed in the following ways:
1. NetMHCpan-v2.8 (Hoof et al., 2009) affinity: the log of the hit:decoy ratio was calculated for logarithmically spaced affinity bins and the overall curve was smoothed monotonically using the isoreg( ) function in R(Team, 2014). This log-ratio value was used rather than nM affinity directly.
2. NetMHCStabPan (Jørgensen et al., 2014) stability: half-lives were used directly since they were normally distributed
3. RNA-Seq Expression: the log of the hit:decoy ratio was calculated for logarithmically spaced expression bins and the overall curve was smoothed monotonically using isoreg( ). This log ratio was used rather than the TPM values directly.
4. Protein Expression: “iBAQ” values (calculated by summing the intensities of observed peptides for a given gene by the theoretical count of tryptic peptides in the gene (Ishihama et al., 2005)) were logtransformed (with zeros set to one tenth the minimum observed iBAQ value).
5. Cleavability scores. A logistic model (described in next section) was built to distinguish MS peptides from decoys (using external data sets) and applied to the B721.221 data (for more details, see next section). The resulting predicted probabilities were then logit transformed. (Logit-transformed NetChop scores were also used for comparison).
6. Localization: Seven dummy (0/1) variables were created to encode the various possible cellular localizations (defined by Uniprot as previously described).

All 63 possible subsets of the 6 variables were evaluated for each allele according the PPVmetric (Table 3). PPVs were averaged across all alleles (shown for select variable combinations in FIG. 5F). In addition, Applicants found the order of variable addition that yielded the most PPV improvement soonest and determined the incremental improvement associated with each variable, considering this as its “explanatory contribution” (FIG. 5H).

An MS-based cleavability predictor was developed by training on previously published MS data sets that profiled melanomas(Bassani-Sternberg et al., 2016), peripheral blood, and the C1R cell line(Caron et al., 2015). To create a set of negative examples, each MS-observed peptide was first mapped to all possible lengthmatched peptides in the proteome that a) have identical amino acids in the N1, N2, C2, and C1 positions and b) are not observed as positive training examples. Among these candidate negative examples (typically hundreds), ten were selected at random (with replacement) using a probability weight proportional to the count of positive training examples mapping to the source transcript. This approach was taken to ensure that targets and decoys would be drawn from a similar set of source genes and resulted in a training set with 10 negative examples per positive example. Training was based on an encoding representing amino acid identities and properties (i.e. isA, isC, isD, isE, isF, isG, isH, isl, isK, isL, isM, isN, isP, isQ, isR, isS, isT, isV, isW, isY, and isBlank plus pKA, volume, and polarity (http://www.proteinsandproteomics.org/content/free/tables_1/table08.pdf)) and included positions U3, U2, U1, N1, N2, N3, C3, C2, C1, D1, D2, and D3 as well as a weighted average of positions U30 . . . U4 (W=1 . . . 27), a weighted average of positions D4 . . . D30 (W=27 . . . 1), and an unweighted average of positions N3 . . . C3. These data were used to train a neural network (2 hidden layers of 50 and 10 nodes; 20% dropout for regularization; keras neural networks library (https://github.com/fchollet/keras)). To eliminate MS bias against cysteines, cysteines in cysteine-containing peptides were converted to serines for the purpose of forward prediction.

### Saturation Analysis

To determine the number of peptides required to build a strong predictor, Applicants carried out saturation analysis by training models with varying numbers of positive training examples (minimum of 15 and maximum the full set of MS-identified peptides) and by measuring PPV on a test set of fixed size. Performance improvement was seen to plateau at several hundred peptides (FIG. 5D), with variation across alleles likely due to the varying complexity of the peptide repertoire per allele. Indeed, complexity score, defined as a decay-weighted average of the entropies at each peptide position, ranked the alleles with strongest performance, HLA-A*01:01,-B*44:03,-B*44:02,-A*29-02, as 1, 2, 3, and 5 of 16 respectively, from least to most complex.

### Predicting External Datasets Using MS-Trained Neural Networks

Performance of the MS-trained models was evaluated on 6 independent external data sources. First, Applicants used a competition dataset of eluted 9mer peptides and non-binders(Zhang et al., 2011). ‘MSIntrinsic’ performed better for 2 of 4 alleles compared to NetMHC-4.0 and NetMHCpan-2.8, even though most of the competition dataset was included in IEDB and likely in NetMHC training (Table 4A). Second, Applicants evaluated the methods using a curated and orthogonal dataset of 52 HIV-1 epitopes (that were associated with 12 HLA alleles from the study) for which T cell responses had been detected in patients (Llano A, Williams, A, Overa, A, Silva-Arrieta, S, Brander, 2013). Applicants evaluated on the set of all HIV 9mer epitopes (excluding any that overlap with the data) mixed with a set of all HIV decoys (all tiled 9-mers across HIV proteins, excluding true HIV epitopes, ˜3000 peptides). After scoring and ranking all peptides, ‘MSIntrinsic’ was able to predict the top-ranked true epitope at the same or higher position compared to NetMHC-4.0 or NetMHCpan-2.8 for 9 of the 12 alleles (Table 4B). Third, Applicants made predictions on 9mer T cell response epitopes retrieved from IEDB (Chowell et al., 2015) by accessing PPV and AUC (Table 4C). To compute PPV, the top 0.1% of the model's predicted peptides were considered true positives. Applicants ruled out 0.01% because Applicants have directly observed more than 1000 9mers for some alleles, and 1% would imply that 100,000 peptides are presented per allele, which is inconsistent with previous biochemical estimates (Walz et al., 2015). Applicants thus define PPV as the fraction of LC-MS/MS peptides found within the model's 0.1% top scoring peptides. In this way, Applicants test how effectively a model calls MS peptides from a background of random peptides (e.g. for n MS-observed 9mer peptides, Applicants mix in 999 n random 9mer decoy peptides from the human genome). Fourth, Applicants predicted HLAbound peptides an independent source of peptides eluted from purified HLA molecules using LC-MS/MS from 7 cell lines that express multiple HLA alleles (Bassani-Sternberg et al., 2015). For each allele that overlapped with the study, Applicants first excluded peptides that were predicted to bind other alleles (<150 nM by NetMHCpan-2.8) but not the allele of interest (>1000 nM), and then added 999 n decoys (FIG. 5E). Finally, Applicants evaluated the models on the soluble HLA single-allele mass spectrometry dataset generated by Trolle and colleagues. Similarly, 999 n decoys were introduced to the identified peptides and PPV and AUC were evaluated. Since the data is allele-specific, there was no uncertainty in assigning peptides to alleles (FIG. 5E). To determine whether NetMHC's weaker performance related to MS bias, a second set of NetMHCbased predictions were made by B721.221-trained logistic regressions based on log NetMHC affinity, ESP observability, and count of cysteines. Expression data from for the cell lines in the two studies was downloaded from CCLE and ENCODE.

### Tables

Lengthy table referenced here
US20190346442A1-20191114-T00001
Please refer to the end of the specification for access instructions.

Lengthy table referenced here
US20190346442A1-20191114-T00002
Please refer to the end of the specification for access instructions.

Lengthy table referenced here
US20190346442A1-20191114-T00003
Please refer to the end of the specification for access instructions.

Lengthy table referenced here
US20190346442A1-20191114-T00004
Please refer to the end of the specification for access instructions.

Lengthy table referenced here
US20190346442A1-20191114-T00005
Please refer to the end of the specification for access instructions.

Lengthy table referenced here
US20190346442A1-20191114-T00006
Please refer to the end of the specification for access instructions.

Lengthy table referenced here
US20190346442A1-20191114-T00007
Please refer to the end of the specification for access instructions.

Table 4: Machine Learning model performance for individual HLA alleles with available stability predictions. A. Internal Evalaution. AUC and PPV machine learning model performance for individual HLA alleles as evaluated on the LC-MS/MS data set. B. PPV and AUC evaluation results on DFRMLI competition data set along with the number of binders and non-binders per allele. C. Due to the small size of the data set, the rank of each evaluated HIV epitope are shown for ‘MSIntrinsic’, NetMHC-4.0 and NetMHCpan-2.8 predictors, instead of PPV and AUC evaluations.

TABLE 4 A

| | NetMHC-4.0 | | NetMHCPan-2.8 | | MS Ensemble 1 | | MS Ensemble 2 | |
|---|---|---|---|---|---|---|---|---|
| | PPV 0.1% | AUC | PPV 0.1% | AUC | PPV 0.1% | AUC | PPV 0.1% | AUC |
| A0101 | 0.547 | 0.997 | 0.582 | 0.998 | 0.702 | 0.999 | 0.746 | 0.999 |
| A0201 | 0.180 | 0.990 | 0.188 | 0.991 | 0.410 | 0.994 | 0.550 | 0.995 |
| A0203 | 0.167 | 0.980 | 0.188 | 0.979 | 0.408 | 0.992 | 0.499 | 0.994 |
| A0204 | NA | NA | 0.140 | 0.989 | 0.434 | 0.996 | 0.572 | 0.996 |
| A0207 | 0.135 | 0.969 | 0.141 | 0.981 | 0.453 | 0.995 | 0.556 | 0.996 |
| A0301 | 0.222 | 0.981 | 0.249 | 0.982 | 0.439 | 0.987 | 0.533 | 0.989 |
| A2402 | 0.291 | 0.994 | 0.297 | 0.994 | 0.469 | 0.996 | 0.579 | 0.997 |
| A2902 | 0.313 | 0.995 | 0.351 | 0.996 | 0.498 | 0.997 | 0.607 | 0.998 |
| A3101 | 0.174 | 0.987 | 0.198 | 0.987 | 0.422 | 0.990 | 0.549 | 0.994 |
| A6802 | 0.253 | 0.942 | 0.265 | 0.948 | 0.472 | 0.964 | 0.560 | 0.971 |
| B3501 | 0.229 | 0.976 | 0.231 | 0.979 | 0.429 | 0.990 | 0.591 | 0.994 |
| B4402 | 0.292 | 0.993 | 0.290 | 0.993 | 0.596 | 0.996 | 0.684 | 0.997 |
| B4403 | 0.361 | 0.991 | 0.251 | 0.991 | 0.590 | 0.994 | 0.664 | 0.994 |
| B5101 | 0.365 | 0.980 | 0.374 | 0.981 | 0.527 | 0.987 | 0.623 | 0.993 |
| B5401 | 0.386 | 0.979 | 0.420 | 0.977 | 0.506 | 0.987 | 0.578 | 0.988 |
| B5701 | 0.306 | 0.962 | 0.321 | 0.968 | 0.418 | 0.977 | 0.537 | 0.983 |
| AVG | 0.281 | 0.981 | 0.280 | 0.983 | 0.486 | 0.990 | 0.589 | 0.992 |

TABLE 4 B

| | Competition Data | | | | | | | |
|---|---|---|---|---|---|---|---|---|
| | | | PPV | | | | | |
| | Counts | | netMHC-4.0 | netMHCPan-2.8 | MS intrinsic | AUC | | |
| | #Binders | #Total | (#missed) | (#missed) | (#missed) | netMHC-4.0 | netMHCPan-2.8 | MS intrinsic |
| A*02:01 | 971 | 5811 | 0.818 (177) | 0.825 (170) | 0.841 (154) | 0.966 | 0.957 | 0.964 |
| B*35:01 | 152 | 717 | 0.862 (21) | 0.862 (21) | 0.855 (22) | 0.970 | 0.969 | 0.964 |
| B*44:03 | 86 | 256 | 0.941 (5) | 0.894 (9) | 0.894 (9) | 0.994 | 0.980 | 0.971 |
| B*57:01 | 133 | 1715 | 0.902 (13) | 0.887 (15) | 0.962 (5) | 0.989 | 0.989 | 0.993 |

TABLE 4C

| | HIV epitope ranks | | |
|---|---|---|---|
| | | NetMHC | NetMHC |
| | MS | 4.0 | Pan-2.8 |
| A0101 | 121 | 283 | 272 |
| A0201 | 1 | 2 | 3 |
| | 2 | 12 | 14 |
| | 16 | 18 | 16 |
| | 19 | 20 | 33 |
| | 40 | 37 | 62 |
| | 106 | 110 | 70 |
| | 147 | 135 | 147 |
| | 156 | 222 | 201 |
| A0207 | 1 | 3 | 1 |
| A0301 | 4 | 2 | 1 |
| | 14 | 3 | 4 |
| | 15 | 5 | 20 |
| | 24 | 12 | 24 |
| | 29 | 17 | 30 |
| | 31 | 27 | 36 |
| | 66 | 73 | 92 |
| | 73 | 84 | 100 |
| A2402 | 1 | 5 | 6 |
| | 5 | 16 | 45 |
| A2902 | 1 | 4 | 7 |
| A6802 | 5 | 78 | 63 |
| | 29 | 173 | 145 |
| | 167 | 252 | 150 |
| B3501 | 1 | 1 | 1 |
| | 8 | 3 | 2 |
| | 10 | 12 | 13 |
| | 11 | 21 | 21 |
| | 21 | 27 | 25 |
| | 37 | 33 | 26 |
| | 39 | 67 | 31 |
| | 242 | 70 | 38 |
| | 401 | 84 | 82 |
| | 613 | 909 | 1653 |
| B4403 | 583 | 224 | 216 |
| B5101 | 7 | 4 | 1 |
| | 31 | 19 | 2 |
| | 34 | 20 | 3 |
| B5401 | 1 | 3 | 1 |
| | 4 | 9 | 13 |
| | 56 | 100 | 17 |
| B5701 | 1 | 6 | 6 |
| | 2 | 8 | 7 |
| | 11 | 14 | 8 |
| | 12 | 21 | 20 |
| | 31 | 25 | 24 |
| | 33 | 31 | 30 |
| | 43 | 32 | 40 |
| | 48 | 42 | 41 |
| | 176 | 180 | 107 |
| | 305 | 346 | 288 |

TABLE 5
List of cysteinylated peptides identified
from all mono-ellelic cell lines

| | | Cys-Cys |
|---|---|---|
| Allele | Sequence | Position |
| A0101 | KTDIQIALPSGcY | 12 |
| A0101 | FTDGITNKLIGcY | 12 |
| A0101 | VTDDLVcLVY | 7 |
| A0101 | FSEAcWEVY | 5 |
| A0101 | cLEPQITPSYY | 1 |
| A0101 | TTDcSFIFLY | 4 |
| A0101 | TTDcLQILAY | 4 |
| A0101 | YSDLASLGcISRY | 9 |
| A0101 | HTDIQEYIGcY | 10 |
| A0101 | ESENVVcHFY | 7 |
| A0101 | YSAEPLPELcY | 10 |
| A0101 | NSELScQLY | 6 |
| A0101 | ATDSGFEILPcNRY | 11 |
| A0101 | ALDDFTIcYF | 8 |
| A0101 | YSDFFTDcY | 8 |
| A0101 | LSELAALcY | 8 |
| A0101 | YLDLLLGNcY | 9 |
| A0101 | WSEPQSLcY | 8 |
| A0101 | KLDTLcDLY | 6 |
| A0101 | KSDIWSLGcILY | 9 |
| A0101 | FSELSAcLY | 7 |
| A0101 | cSDKmSLLLVY | 1 |
| A0101 | cLDHVISYY | 1 |
| A0101 | LLDDmNHcY | 8 |
| A0101 | SSDQcAVQLFY | 5 |
| A0201 | ALLGAGcDPEL | 7 |
| A0201 | ALLEDScHYL | 7 |
| A0201 | SLFPHAIcL | 8 |
| A0201 | YLLDIGcGTGL | 7 |
| A0201 | ILFDcPGQIEL | 5 |
| A0201 | cLIKEVDIYTV | 1 |
| A0201 | GLLPGcVYHV | 6 |
| A0201 | TLVTWLQcV | 8 |
| A0201 | TLVTWLQcV | 8 |
| A0201 | YLSDPcPGLYL | 6 |
| A0201 | SLMEESGIcKV | 9 |
| A0201 | KLVDcIIEV | 5 |
| A0201 | AIIDGKIFcV | 9 |
| A0201 | GLYDGPVcEV | 8 |
| A0201 | ALSEAMGLFcL | 10 |
| A0201 | ALIDEQILcV | 9 |
| A0201 | FLFDcPGQVEL | 5 |
| A0201 | AIIDGKIFcV | 9 |
| A0201 | SLLAcEFLL | 5 |
| A0201 | SLVYLcYTV | 6 |
| A0201 | SLMEESGIcKV | 9 |
| A0201 | LIDEQILcV | 8 |
| A0201 | ALIDEQILcV | 9 |
| A0201 | GLFGVPLcL | 8 |
| A0201 | ILDcIYNEV | 4 |
| A0201 | ILDcIYNEV | 4 |
| A0201 | cLYELPENIRV | 1 |
| A0201 | cLYEIYPEL | 1 |
| A0201 | QLQPTDALLcV | 10 |
| A0201 | RLMQGDEIcL | 9 |
| A0201 | SLAPVLcGI | 7 |
| A0201 | ALVDcSVAL | 5 |
| A0201 | SLIEYcIEL | 6 |
| A0201 | LLPDIVTcV | 8 |
| A0201 | SLLPADcQIHL | 7 |
| A0201 | ALTDVILcV | 8 |
| A0201 | YLLDIGcGTGL | 7 |
| A0201 | ALSEAMGLFcL | 10 |
| A0201 | SLLDcTFRL | 5 |
| A0201 | SLIEYcIEL | 6 |
| A0201 | QIMDYLLcL | 8 |
| A0201 | SLEENLPcI | 8 |
| A0201 | ALSQLVPcV | 8 |
| A0201 | ALIDEQILcV | 9 |
| A0201 | HILEcEFYL | 5 |
| A0201 | ALSEAMGLFcL | 10 |
| A0201 | LIDEQILcV | 8 |
| A0201 | ALSEAMGLFcL | 10 |
| A0201 | LLPDIVTcV | 8 |
| A0201 | AIIDGKIFcV | 9 |
| A0201 | SLAPVLcGI | 7 |
| A0201 | TLVTWLQcV | 8 |
| A0201 | GLLDcPIFL | 5 |
| A0201 | SLLEWcQEV | 6 |
| A0201 | RLLEQGcTDFTV | 7 |
| A0201 | TLWVDPcEV | 7 |
| A0201 | GLYDGPVcEV | 8 |
| A0201 | cLYEIYPEL | 1 |
| A0201 | SLMEESGIcKV | 9 |
| A0201 | TLcDLYETL | 3 |
| A0201 | FLGcIGAVNEV | 4 |
| A0101 | KTDIQIALPSGcY | 12 |
| A0201 | KLFADAGLVcI | 10 |
| A0201 | YLSDPcPGLYL | 6 |
| A0201 | cLYPHIDKQYL | 1 |
| A0201 | YMLPDGTYcL | 9 |
| A0201 | LLDGcRIYL | 5 |
| A0201 | YLYcGQEGL | 4 |
| A0201 | KLVDcIIEV | 5 |
| A0201 | SLSTcIPAI | 5 |
| A0201 | ALTDVILcV | 8 |
| A0201 | SLLDcTFRL | 5 |
| A0203 | ILAPcKLETV | 5 |
| A0203 | VLFDHVGcL | 8 |
| A0203 | YLFDRNGVcL | 9 |
| A0203 | TVYGGYLcSV | 8 |
| A0203 | YMFcELVTGV | 4 |
| A0203 | MLYGTGPLcSV | 9 |
| A0203 | VLKDcIVHL | 5 |
| A0203 | FLSYcPGmGV | 5 |
| A0203 | GLFAGPcKV | 7 |
| A0203 | SLFTcEPITV | 5 |
| A0203 | YLFKcPQSV | 5 |
| A0203 | AVYEGHVScV | 9 |
| A0203 | ALYcEFINRV | 4 |
| A0203 | RLFTDVIIcV | 9 |
| A0203 | TVYGGYLcSV | 8 |
| A0203 | SLKTLLEcV | 8 |
| A0203 | RMIKEKLcYV | 8 |
| A0203 | VLFScHVRKV | 5 |
| A0203 | SLASFcFSHI | 6 |
| A0203 | SLHDALcVV | 7 |
| A0203 | SLKYQTRcI | 8 |
| A0203 | cLMGKGMKRV | 1 |
| A0204 | TLLEALDcI | 8 |
| A0204 | GVTAIIFcV | 8 |
| A0204 | RLLDVLcEm | 7 |
| A0204 | ALAcWEWLL | 4 |
| A0204 | AILPSIFcL | 8 |
| A0204 | ILLGNYcVAV | 7 |
| A0204 | FLFTTPcRL | 7 |
| A0204 | VAGAKVAKGQPLc | 13 |
| | VLSAMK | |
| A0207 | FIDDLADLScL | 10 |
| A0207 | LIDDLQHcL | 8 |
| A0207 | YLDcGDLSNAL | 4 |
| A0207 | LLVPVIcQI | 7 |
| A0207 | YIPTFIcSV | 7 |
| A0207 | LVDGQIFcL | 8 |
| A0207 | SVDEDFcHYL | 7 |
| A0207 | VLPETcEEL | 6 |
| A0207 | ALEEYVIcV | 8 |
| A0207 | ALDYIVPcM | 8 |
| A0207 | TLDNIFLcV | 8 |
| A0207 | MLDQINScL | 8 |
| A0207 | ALPDWcEQL | 6 |
| A0207 | FLDDFIAcV | 8 |
| A0207 | SVDSHFcHL | 7 |
| A0207 | AVLDVLLcL | 8 |
| A0207 | YVDPSPDYcL | 9 |
| A0207 | TLPEVVGcEL | 8 |
| A0207 | FLnHcLEHL | 5 |
| A0207 | HLPDVcVNL | 6 |
| A0207 | mIDDTYQcL | 8 |
| A0207 | ALDYIVPcM | 8 |
| A0207 | FVDcPGHDIL | 4 |
| A0207 | TLDSIcDSL | 6 |
| A0207 | VLPDEIcNL | 7 |
| A0207 | AVFGLTTcI | 8 |
| A0207 | AInNcRSI | 5 |
| A0301 | ILNSHcFAR | 6 |
| A0301 | RIKEIFcPK | 7 |
| A0301 | KLYDLVAGSNcLK | 11 |
| A0301 | VVcEYIVKK | 3 |
| A0301 | RLFcVGFTKK | 4 |
| A0301 | RLADKSVLVcK | 10 |
| A0301 | QVLcIPSWMAK | 4 |
| A0301 | TMcPHILRY | 3 |
| A0301 | AVWDTcLEY | 6 |
| A0301 | RVFFPLcGK | 7 |
| A0301 | TMcPHILRY | 3 |
| A0301 | RLFFHcSQY | 6 |
| A0301 | KLFTEVEGTcTGK | 10 |
| A0301 | TLYISEcLK | 7 |
| A0301 | RVNKLIcVK | 7 |
| A0301 | RLFcVGFTK | 4 |
| A0301 | RVKcNTDDTIGDLK | 4 |
| A0301 | TLcKPLVPR | 3 |
| A0301 | TLYISEcLKK | 7 |
| A0301 | KVcNPIITKLY | 3 |
| A0301 | RLFQcLLHR | 5 |
| A0301 | ILYcIPLRY | 4 |
| A0301 | VLYSLQIcK | 8 |
| A0301 | RVFQEcLTY | 6 |
| A0301 | RLPSATLcY | 8 |
| A0301 | GLYHGQVLcK | 9 |
| A0301 | RITEWVSVcK | 9 |
| A0301 | AIFPATFcQK | 8 |
| A0301 | ILNSHcFAR | 6 |
| A0301 | cLYPRFVQR | 1 |
| A0301 | IVRTGGHFIcK | 10 |
| A0301 | TVLcQPTGGK | 4 |
| A0301 | RIYSGENPFAcK | 11 |
| A2402 | HWAEIcETF | 6 |
| A2402 | SYVcPDLVKEF | 4 |
| A2402 | DYLPSFcKW | 7 |
| A2402 | IYLAPGDYHcF | 10 |
| A2402 | EYQLIDcAQYF | 7 |
| A2402 | RYFIPVScF | 8 |
| A2402 | NYVVIGTcTF | 8 |
| A2402 | IYIDAScLTW | 7 |
| A2402 | LYGELcALLF | 6 |
| A2402 | VYIPcIYVL | 5 |
| A2402 | HYQDVScLQF | 7 |
| A2402 | SYLcNVTLF | 4 |
| A2402 | LYLEcSAKF | 5 |
| A2402 | AYITGLcFI | 7 |
| A2402 | RYLPQcSYF | 6 |
| A2402 | IYQWIcDNF | 6 |
| A2402 | cYIKILHQL | 1 |
| A2402 | VYADTcFSTI | 6 |
| A2402 | TYDPFHNcW | 8 |
| A2402 | cYLLQVDEF | 1 |
| A2402 | VYQPVTTEcF | 9 |
| A2402 | IYSTLVTcVTF | 8 |
| A2402 | RYPcFFNTL | 4 |
| A2402 | QFIDKPVcF | 8 |
| A2402 | LYDPcTVMF | 5 |
| A2402 | SYIISGcLF | 7 |
| A2402 | cYVLFSYSF | 1 |
| A2402 | cYVQPQWVF | 1 |
| A2402 | AYLEcIERITF | 5 |
| A2402 | VYLPcLQNI | 5 |
| A2402 | IWPEKSFcL | 8 |
| A2402 | SYIHYFHcL | 8 |
| A2402 | AYTDcIPQL | 5 |
| A2402 | TYGcTWEF | 4 |
| A2402 | RYRPDMPcF | 8 |
| A2402 | IYLDSVMcL | 8 |
| A2402 | YYAVcQNLL | 5 |
| A2402 | RYRPDMPcFLL | 8 |
| A2402 | IYLGQLEcF | 8 |
| A2402 | cYAELGTTI | 1 |
| A2402 | QYGTFcEKF | 6 |
| A2902 | ALLQcALLY | 5 |
| A2902 | FLPELIWcY | 8 |
| A2902 | QLQcVVIFVF | 4 |
| A3101 | cVNQFIISR | 1 |
| A6802 | MTSNIVQcL | 8 |
| A6802 | EAAcLIVSV | 4 |
| A6802 | NTSAIVIcI | 8 |
| A6802 | ETIYIVGGcL | 9 |
| A6802 | AVYFHQHSILAcKI | 12 |
| B3501 | SPTcLTLIY | 4 |
| B3501 | VPVSVVEcF | 8 |
| B3501 | FANPEDcVAF | 7 |
| B3501 | LPFDcTQAL | 5 |
| B3501 | HAcGVIATI | 3 |
| B3501 | FPYKNcKTDF | 6 |
| B3501 | FPQEFIIcF | 8 |
| B3501 | LPVDFVEcL | 8 |
| B5101 | LPYcPGKTLVV | 4 |
| B5101 | FPFGcPPTV | 5 |
| B5101 | cPFTGNVSI | 1 |
| B5101 | FPFGcPPTV | 5 |
| B5101 | DPLQQIcKI | 7 |
| B5401 | FALNPDILcSA | 9 |
| B5401 | cPFSSKFFSA | 1 |
| B5401 | mPLQTGTAQIcA | 11 |
| B5701 | HSQVcSILW | 5 |
| B5701 | HTIGcNAVSW | 5 |
| B5701 | STLPVScAW | 7 |
| B5701 | ATLIISPSSIcHQW | 11 |
| B5701 | ISDHEATLRcW | 10 |
| B5701 | GSIDSSIRcW | 9 |
| B5701 | RAFTcDDLFRF | 5 |
| B5701 | RSVNIKEIcW | 9 |

### REFERENCES

1. M. Bassani-Sternberg, S. Pletscher-Frankild, L. J. Jensen, M. Mann, Mass Spectrometry of Human Leukocyte Antigen Class I Peptidomes Reveals Strong Effects of Protein Abundance and Turnover on Antigen Presentation, Mol. Cell. Proteomics 14, 658-673 (2015).

2. D. Hunt, R. Henderson, J. Shabanowitz, K. Sakaguchi, H. Michel, N. Sevilir, A. Cox, E. Appella, V. Engelhard, Characterization of peptides bound to the class I MHC molecule HLA-A2.1 by mass spectrometry, Science 255, 1261-1263 (1992).

3. H.-G. Rammensee, T. Friede, S. Stevanovié, MHC ligands and peptide motifs: first listing, Immunogenetics 41, 178-228 (1995).

4. H.-G. Rammensee, J. Bachmann, N. P. N. Emmerich, O. A. Bachor, S. Stevanovié, SYFPEITHI: database for MHC ligands and peptide motifs, Immunogenetics 50, 213-219 (1999).

5. R. Vita, J. A. Overton, J. A. Greenbaum, J. Ponomarenko, J. D. Clark, J. R. Cantrell, D. K. Wheeler, J. L. Gabbard, D. Hix, A. Sette, B. Peters, The immune epitope database (IEDB) 3.0, Nucleic Acids Res. 43, D405-D412 (2015).

6. J. Robinson, J. A. Halliwell, J. D. Hayhurst, P. Flicek, P. Parham, S. G. E. Marsh, The IPD and IMGT/HLA database: allele variant databases, Nucleic Acids Res. 43, D423-D431 (2015).

7. I. Hoof, B. Peters, J. Sidney, L. E. Pedersen, A. Sette, O. Lund, S. Buus, M. Nielsen, NetMHCpan, a method for MHC class I binding prediction beyond humans, Immunogenetics 61, 1-13 (2009).

8. C. Lundegaard, K. Lamberth, M. Harndahl, S. Buus, O. Lund, M. Nielsen, NetMHC-3.0: accurate web accessible predictions of human, mouse and monkey MHC class I affinities for peptides of length 8-11, Nucleic Acids Res. 36, W509-W512 (2008).

9. E. Boen, A. R. Crownover, M. Mcllhaney, A. J. Korman, J. Bill, Identification of T Cell Ligands in a Library of Peptides Covalently Attached to HLA-DR4, J. Immunol. 165, 2040-2047 (2000).

10. M. V. Larsen, C. Lundegaard, K. Lamberth, S. Buus, O. Lund, M. Nielsen, Large-scale validation of methods for cytotoxic T-lymphocyte epitope prediction, BMC Bioinformatics 8, 424-424 (2007).

11. E. Caron, D. J. Kowalewski, C. C. Koh, T. Sturm, H. Schuster, R. Aebersold, Analysis of MHC immunopeptidomes using mass spectrometry, Mol. Cell. Proteomics (2015), doi:10.1074/mcp.0115.052431.

12. Y. Shimizu, R. DeMars, Production of human cells expressing individual transferred HLA-A,-B,-C genes using an HLA-A,-B,-C null human cell line., J. Immunol. 142, 3320-3328 (1989).

13. Y. Shimizu, B. Koller, D. Geraghty, H. Orr, S. Shaw, P. Kavathas, R. DeMars, Transfer of cloned human class I major histocompatibility complex genes into HLA mutant human lymphoblastoid cells., Mol. Cell. Biol. 6, 1074-1087 (1986).

14. K. V. Ruggles, Z. Tang, X. Wang, H. Grover, M. Askenazi, J. Teubl, S. Cao, M. D. McLellan, K. R. Clauser, D. L. Tabb, P. Mertins, R. Slebos, P. Erdmann-Gilmore, S. Li, H. P. Gunawardena, L. Xie, T. Liu, J.-Y. Zhou, S. Sun, K. A. Hoadley, C. M. Perou, X. Chen, S. R. Davies, C. A. Maher, C. R. Kinsinger, K. D. Rodland, H. Zhang, Z. Zhang, L. Ding, R. R. Townsend, H. Rodriguez, D. Chan, R. D. Smith, D. C. Liebler, S. A. Carr, S. Payne, M. J. Ellis, D. Fenyo, An analysis of the sensitivity of proteogenomic mapping of somatic mutations and novel splicing events in cancer, Mol. Cell. Proteomics (2015), doi:10.1074/mcp.M115.056226.

15. F. Boisgerault, I. Khalil, V. Tieng, F. Connan, T. Tabary, J. H. Cohen, J. Choppin, D. Charron, A. Toubert, Definition of the HLA-A29 peptide ligand motif allows prediction of potential T-cell epitopes from the retinal soluble antigen, a candidate autoantigen in birdshot retinopathy., Proc. Natl. Acad. Sci. U.S.A. 93, 3466-3470 (1996).

16. P. Guasp, C. Alvarez-Navarro, P. Gomez-Molina, A. Martín-Esteban, M. Marcilla, E. Barnea, A. Admon, J. A. López de Castro, The Peptidome of Behçet's Disease-Associated HLA-B*51:01 Includes Two Subpeptidomes Differentially Shaped by Endoplasmic Reticulum Aminopeptidase 1, Arthritis Rheumatol. 68, 505-515 (2016).

17. K. J. M. Jeffery, A. A. Siddiqui, M. Bunce, A. L. Lloyd, A. M. Vine, A. D. Witkover, S. Izumo, K. Usuku, K. I. Welsh, M. Osame, C. R. M. Bangham, The Influence of HLA Class I Alleles and Heterozygosity on the Outcome of Human T Cell Lymphotropic Virus Type I Infection, J. Immunol. 165, 7278-7284 (2000).

18. M. Berg, A. Parbel, H. Pettersen, D. Fenyo, L. Björkesten, Detection of artifacts and peptide modifications in liquid chromatography/mass spectrometry data using two-dimensional signal intensity map data visualization, Rapid Commun. Mass Spectrom. 20, 1558-1562 (2006).

19. Y. Kim, J. Sidney, C. Pinilla, A. Sette, B. Peters, Derivation of an amino acid similarity matrix for peptide:MHC binding and its application as a Bayesian prior, BMC Bioinformatics 10, 1-11 (2009).

20. E. Milner, L. Gutter-Kapon, M. Bassani-Strenberg, E. Barnea, I. Beer, A. Admon, The Effect of Proteasome Inhibition on the Generation of the Human Leukocyte Antigen (HLA) Peptidome, Mol. Cell. Proteomics 12, 1853-1864 (2013).

21. D. Fruci, P. Giacomini, M. R. Nicotra, M. Forloni, R. Fraioli, L. Saveanu, P. van Endert, P. G. Natali, Altered expression of endoplasmic reticulum aminopeptidases ERAP1 and ERAP2 in transformed non-lymphoid human tissues, J. Cell. Physiol. 216, 742-749 (2008).

22. L. Saveanu, O. Carroll, V. Lindo, M. Del Val, D. Lopez, Y. Lepelletier, F. Greer, L. Schomburg, D. Fruci, G. Niedermann, P. M. van Endert, Concerted peptide trimming by human ERAP1 and ERAP2 aminopeptidase complexes in the endoplasmic reticulum, Nat Immunol 6, 689-697 (2005).

23. C. Keşmir, A. K. Nussbaum, H. Schild, V. Detours, S. Brunak, Prediction of proteasome cleavage motifs by neural networks, Protein Eng. 15, 287-296 (2002).

24. M. Nielsen, C. Lundegaard, O. Lund, C. Keşmir, The role of the proteasome in generating cytotoxic T-cell epitopes: insights obtained from improved predictions of proteasomal cleavage, Immunogenetics 57, 33-41 (2005).

25. P. Saxová, S. Buus, S. Brunak, C. Keşmir, Predicting proteasomal cleavage sites: a comparison of available methods, Int. Immunol. 15, 781-787 (2003).

26. K. L. Rock, D. J. Farfán-Arribas, J. D. Colbert, A. L. Goldberg, Re-examining class-I presentation and the DRiP hypothesis, Trends Immunol. 35, 144-152 (2014).

27. J. W. Yewdell, DRiPs solidify: progress in understanding endogenous MHC class I antigen processing, Trends Immunol. 32, 548-558 (2011).

28. D. Bourdetsky, C. E. H. Schmelzer, A. Admon, The nature and extent of contributions by defective ribosome products to the HLA peptidome, Proc. Natl. Acad. Sci. 111, E1591-E1599 (2014).

29. C. McMurtrey, T. Trolle, T. Sansom, S. G. Remesh, T. Kaever, W. Bardet, K. Jackson, R. McLeod, A. Sette, M. Nielsen, D. M. Zajonc, I. J. Blader, B. Peters, W. Hildebrand, M. S. Gilmore, Ed. Toxoplasma gondii peptide ligands open the gate of the HLA class I binding groove, eLife 5, e12556 (2016).

30. T. Trolle, C. P. McMurtrey, J. Sidney, W. Bardet, S. C. Osborn, T. Kaever, A. Sette, W. H. Hildebrand, M. Nielsen, B. Peters, The Length Distribution of Class I-Restricted T Cell Epitopes Is Determined by Both Peptide Supply and MHC Allele-Specific Binding Preference, J. Immunol. (2016), doi:10.4049/jimmuno1.1501721.

31. M. Andreatta, M. Nielsen, Gapped sequence alignment using artificial neural networks: application to the MHC class I system, Bioinformatics 32 (2016), doi:10.1093/bioinformatics/btv639.

32. M. Harndahl, M. Rasmussen, G. Roder, I. Dalgaard Pedersen, M. Sorensen, M. Nielsen, S. Buus, Peptide-MHC class I stability is a better predictor than peptide affinity of CTL immunogenicity, Eur. J. Immunol. 42, 1405-1416 (2012).

33. K. W. Jørgensen, M. Rasmussen, S. Buus, M. Nielsen, NetMHCstab—predicting stability of peptide-MHC-I complexes; impacts for cytotoxic T lymphocyte epitope discovery, Immunology 141, 18-26 (2014).

34. M. Harndahl, M. Rasmussen, G. Roder, S. Buus, Real-time, High-Throughput Measurements of Peptide-MHC-I Dissociation Using a Scintillation Proximity Assay, J. Immunol. Methods 374, 5-12 (2011).

35. R. D. Bremel, E. J. Homan, An integrated approach to epitope analysis I: Dimensional reduction, visualization and prediction of MHC binding using amino acid principal components and regression approaches, Immunome Res. 6, 7-7 (2010).

36. D. O. R. Tones P. Rondón-Villarreal, Peptides: Calculate indices and theoretical physicochemical properties of peptides and protein sequences. (2014; CRAN.R-project.org/package=Peptides).

37. G. L. Zhang, H. H. Lin, D. B. Keskin, E. L. Reinherz, V. Brusic, Dana-Farber repository for machine learning in immunology, High-Throughput Methods Immunol. Mach. Learn. Autom. 374, 18-25 (2011).

38. Llano A, Williams, A, Overa, A, Silva-Arrieta, S, Brander, Best-Characterized HIV-1 CTL Epitopes: The 2013 Update, HIV Molecular Immunology , 3-25 (2013).

39. E. Lorente, S. Infantes, E. Barnea, I. Beer, A. Barriga, N. Garcia-Medel, F. Lasala, M. Jimenez, A. Admon, D. Lopez, Diversity of Natural Self-Derived Ligands Presented by Different HLA Class I Molecules in Transporter Antigen Processing-Deficient Cells, PLoS ONE 8, e59118 (2013).

40. G. P. M. Mommen, C. K. Frese, H. D. Meiring, J. van Gaans-van den Brink, A. P. J. M. de Jong, C. A. C. M. van Els, A. J. R. Heck, Expanding the detectable HLA peptide repertoire using electron-transfer/higher-energy collision dissociation (EThcD), Proc. Natl. Acad. Sci. 111, 4507-4512(2014).

41. H.-C. Guo, T. S. Jardetzky, T. P. J. Garrettt, W. S. Lane, J. L. Strominger, D. C. Wiley, Different length peptides bind to HLA-Aw68 similarly at their ends but bulge out in the middle, Nature 360, 364-366 (1992).

42. F. E. Tynan, S. R. Burrows, A. M. Buckle, C. S. Clements, N. A. Borg, J. J. Miles, T. Beddoe, J. C. Whisstock, M. C. Wilce, S. L. Silins, J. M. Burrows, L. Kjer-Nielsen, L. Kostenko, A. W. Purcell, J. McCluskey, J. Rossjohn, T cell receptor recognition of a “super-bulged” major histocompatibility complex class I-bound peptide, Nat Immunol 6, 1114-1122 (2005).

43. H. D. Hickman, A. D. Luis, R. Buchli, S. R. Few, M. Sathiamurthy, R. S. VanGundy, C. F. Giberson, W. H. Hildebrand, Toward a Definition of Self: Proteomic Evaluation of the Class I Peptide Repertoire, J. Immunol. 172, 2944-2952 (2004).

44. E. Milner, E. Barnea, I. Beer, A. Admon, The Turnover Kinetics of Major Histocompatibility Complex Peptides of Human Cancer Cells*, Mol. Cell. Proteomics 5, 357-365 (2006).

45. M. M. Gubin, X. Zhang, H. Schuster, E. Caron, J. P. Ward, T. Noguchi, Y. Ivanova, J. Hundal, C. D. Arthur, W.-J. Krebber, G. E. Mulder, M. Toebes, M. D. Vesely, S. S. K. Lam, A. J. Korman, J. P. Allison, G. J. Freeman, A. H. Sharpe, E. L. Pearce, T. N. Schumacher, R. Aebersold, H.-G. Rammensee, C. J. M. Melief, E. R. Mardis, W. E. Gillanders, M. N. Artyomov, R. D. Schreiber, Checkpoint blockade cancer immunotherapy targets tumour-specific mutant antigens, Nature 515, 577-581 (2014).

46. C. Linnemann, M. M. van Buuren, L. Bies, E. M. E. Verdegaal, R. Schotte, J. J. A. Calis, S. Behjati, A. Velds, H. Hilkmann, D. el Atmioui, M. Visser, M. R. Stratton, J. B. A. G. Haanen, H. Spits, S. H. van der Burg, T. N. M. Schumacher, High-throughput epitope discovery reveals frequent recognition of neo-antigens by CD4+ T cells in human melanoma, Nat Med 21, 81-85 (2015).

47. A. L. Pritchard, J. G. Burel, M. A. Neller, N. K. Hayward, J. A. Lopez, M. Fatho, V. Lennerz, T. Wölfel, C. W. Schmidt, Exome Sequencing to Predict Neoantigens in Melanoma, Cancer Immunol. Res. 3, 992-998 (2015).

48. M. Rajasagi, S. A. Shukla, E. F. Fritsch, D. B. Keskin, D. DeLuca, E. Carmona, W. Zhang, C. Sougnez, K. Cibulskis, J. Sidney, K. Stevenson, J. Ritz, D. Neuberg, V. Brusic, S. Gabriel, E. S. Lander, G. Getz, N. Hacohen, C. J. Wu, Systematic identification of personal tumor-specific neoantigens in chronic lymphocytic leukemia, Blood 124, 453-462 (2014).

49. T. N. Schumacher, R. D. Schreiber, Neoantigens in cancer immunotherapy, Science 348, 69-74 (2015).

50. C. Hughes, B. Ma, G. A. Lajoie, in Proteome Bioinformatics, J. S. Hubbard, R. A. Jones, Eds. (Humana Press, Totowa, N.J., 2010), pp. 105-121.

51. B. Ma, Novor: Real-Time Peptide de Novo Sequencing Software, J. Am. Soc. Mass Spectrom. 26, 1885-1894 (2015).

52. J. Ng, N. Bandeira, W.-T. Liu, M. Ghassemian, T. L. Simmons, W. H. Gerwick, R. Linington, P. C. Dorrestein, P. A. Pevzner, Dereplication and de novo sequencing of nonribosomal peptides, Nat Meth 6, 596-599 (2009).

53. P. A. Reche, D. B. Keskin, R. E. Hussey, P. Ancuta, D. Gabuzda, E. L. Reinherz, Elicitation from virus-naive individuals of cytotoxic T lymphocytes directed against conserved HIV-1 epitopes, Med. Immunol. 5, 1-1 (2006).

54. D. B. Keskin, B. Reinhold, S. Y. Lee, G. Zhang, S. Lank, D. O'Connor, R. S. Berkowitz, V. Brusic, S. J. Kim, E. L. Reinherz, Direct identification of an HPV-16 tumor antigen from cervical cancer biopsy specimens, Front. Immunol. 2 (2011), doi:10.3389/fimmu.2011.00075.

55. D. B. Keskin, B. B. Reinhold, G. L. Zhang, A. R. Ivanov, B. L. Karger, E. L. Reinherz, Physical detection of influenza A epitopes identifies a stealth subset on human lung epithelium evading natural CD8 immunity, Proc. Natl. Acad. Sci. U.S.A. 112, 2151-2156 (2015).

56. J. Rappsilber, M. Mann, Y. Ishihama, Protocol for micro-purification, enrichment, pre-fractionation and storage of peptides for proteomics using StageTips, Nat Protoc. 2, 1896-1906 (2007).

57. J. Sidney, S. Southwood, C. Oseroff, M.-F. del Guercio, A. Sette, H. M. Grey, in Current Protocols in Immunology, (John Wiley & Sons, Inc., 2001).

58. J. E. Elias, S. P. Gygi, Target-decoy search strategy for increased confidence in large-scale protein identifications by mass spectrometry. Nat Meth. 4, 207-214 (2007).

59. B. Langmead, S. L. Salzberg, Fast gapped-read alignment with Bowtie 2. Nat Meth. 9, 357-359 (2012).

60. B. Li, C. N. Dewey, RSEM: accurate transcript quantification from RNA-Seq data with or without a reference genome. BMC Bioinformatics. 12, 1-16 (2011).

61. R. C. Team, R: A language and environment for statistical computing. R Foundation for Statistical Computing, Vienna, Austria. 2013 (2014).

62. Kronke et al. “Lenalidomide induces ubiquitination and degradation of CK1α in del(5q) MDS” Nature (2015) 523(7559): 183-8. doi: 10.1038/nature14610. Epub 2015 Jul. 1.

63. Krönke et al. “Lenalidomide causes selective degradation of IKZF1 and IKZF3 in multiple myeloma cells” Science (2014) 343(6168): 301-5. doi: 10.1126/science.1244851. Epub 2013 Nov. 29.

64. Udeshi N. D.; Mani D. R.; Eisenhaure T.; Mertins P.; Jaffe J. D.; Clauser K. R.; Hacohen N.; Carr S. A. “Methods for quantification of in vivo changes in protein ubiquitination following proteasome and deubiquitinase inhibition” Molecular & Cellular Proteomics (2012) 11: 148-59.

65. Fusaro et al. “Prediction of high-responding peptides for targeted protein assays by mass spectrometry” Nature Biotechnology (2009) 27, 190-198.

66. Eyers et al. “CONSeQuence: prediction of reference peptides for absolute quantitative proteomics using consensus machine learning approaches” Mol Cell Proteomics (2011); 10(11):M110.003384. doi: 10.1074/mcp.M110.003384. Epub 2011 Aug. 3.

70. Muntel, J., Boswell, S. A., Tang, S., Ahmed, S., Wapinski, I., Foley, G., Steen, H., and Springer, M. (2015) “Abundance-based Classifier for the Prediction of Mass Spectrometric Peptide Detectability Upon Enrichment (PPA)” Mol. Cell. Proteomics 14, 430-440

71. Searle, B. C., Egertson, J. D., Bollinger, J. G., Stergachis, A. B., and MacCoss, M. J. (2015) “Using Data Independent Acquisition (DIA) to Model High-responding Peptides for Targeted Proteomics Experiments” Mol. Cell. Proteomics 14, 2331-2340.

72. Mommen, G. P. M., Marino, F., Meiring, H. D., Poelen, M. C. M., van Gaans-van den Brink, J. A. M., Mohammed, S., Heck, A. J. R., and van Els, C. A. C. M. (2016) “Sampling From the Proteome to the Human Leukocyte Antigen-DR (HLA-DR) Ligandome Proceeds Via High Specificity” Mol. Cell. Proteomics MCP 15, 1412-1423.

73. Rock, K. L., Farfán-Arribas, D. J., Colbert, J. D., and Goldberg, A. L. (2014) “Re-examining class-I presentation and the DRiP hypothesis” Trends Immunol. 35, 144-152.

74. Bourdetsky, D., Schmelzer, C. E. H., and Admon, A. (2014). “The nature and extent of contributions by defective ribosome products to the HLA peptidome” Proc. Natl. Acad. Sci. 111, E1591-E1599

75. Yewdell, J. W. (2011) “ DRiPs solidify: progress in understanding endogenous MHC class I antigen processing” Trends Immunol. 32, 548-558.

76. Kim, Y., Yewdell, J. W., Sette, A., and Peters, B. (2013) “Positional Bias of MHC Class I Restricted T-Cell Epitopes in Viral Antigens Is Likely due to a Bias in Conservation” PLoS Comput Biol 9, e1002884.

77. Guruprasad, K., Reddy, B. V. B., and Pandit, M. W. (1990) “Correlation between stability of a protein and its dipeptide composition: a novel approach for predicting in vivo stability of a protein from its primary sequence” Protein Eng. 4, 155-161.

78. Behrends, C., Sowa, M. E., Gygi, S. P., and Harper, J. W. (2010) “Network organization of the human autophagy system” Nature 466, 68-76.

79. Christianson, J. C., Olzmann, J. A., Shaler, T. A., Sowa, M. E., Bennett, E. J., Richter, C. M., Tyler, R. E., Greenblatt, E. J., Harper, J. W., and Kopito, R. R. (2012) “Defining human ERAD networks through an integrative mapping strategy” Nat. Cell Biol. 14, 93-105

80. Sowa, M. E., Bennett, E. J., Gygi, S. P., and Harper, J. W. (2009) “Defining the Human Deubiquitinating Enzyme Interaction Landscape” Cell 138, 389-403.

81. Sidney, J., Southwood, S., Oseroff, C., del Guercio, M.-F., Sette, A., and Grey, H. M. (2001). “Measurement of MHC/Peptide Interactions by Gel Filtration” In Current Protocols in Immunology 31:18.3:18.3.1-18.3.19.

82. Nielsen, M., Lundegaard, C., Lund, O., and Kemir, C. (2005) “The role of the proteasome in generating cytotoxic T-cell epitopes: insights obtained from improved predictions of proteasomal cleavage” Immunogenetics 57, 33-41.

83. Li, B., and Dewey, C. N. (2011) “RSEM: accurate transcript quantification from RNA-Seq data with or without a reference genome” BMC Bioinformatics 12, 1-16.

84. Oates, M. E., Romero, P., Ishida, T., Ghalwash, M., Mizianty, M. J., Xue, B., Dosztányi, Z., Uversky, V. N., Obradovic, Z., Kurgan, L., et al. (2013). D(2)P(2): database of disordered protein predictions. Nucleic Acids Res. 41, D508-D516.

85. Eichmann, M., Ru, A., Veelen, P. A., Peakman, M., and Kronenberg-Versteeg, D. (2014) “Identification and characterisation of peptide binding motifs of six autoimmune disease-associated human leukocyte antigen-class I molecules including HLA-B*39:06” Tissue Antigens 84.

86. Theano Development Team (2016) “Theano: A Python framework for fast computation of mathematical expressions” ArXiv E-Prints abs/1605.02688.

87. Bremel, R. D., and Homan, E. J. (2010) “An integrated approach to epitope analysis I: Dimensional reduction, visualization and prediction of MHC binding using amino acid principal components and regression approaches” Immunome Res. 6, 7-7.

88. Osorio, D.; Rondon-Villarreal, P.; Tones, R. “Stability Analysis of Antimicrobial Peptides in Solvation Conditions by Molecular Dynamics” Adv Comp Bio (2014) 232, 127-131.

89. Ishihama, Y., Oda, Y., Tabata, T., Sato, T., Nagasu, T., Rappsilber, J., and Mann, M. (2005) “Exponentially Modified Protein Abundance Index (emPAl) for Estimation of Absolute Protein Amount in Proteomics by the Number of Sequenced Peptides per Protein” Mol. Cell. Proteomics 4, 1265-1272.

90. Chowell, D., Krishna, S., Becker, P. D., Cocita, C., Shu, J., Tan, X., Greenberg, P. D., Klavinskis, L. S., Blattman, J. N., and Anderson, K. S. (2015) “ TCR contact residue hydrophobicity is a hallmark of immunogenic CD8(+) T cell epitopes” Proc. Natl. Acad. Sci. U.S.A. 112, E1754-E1762.

91. Walz, S., Stickel, J. S., Kowalewski, D. J., Schuster, H., Weisel, K., Backert, L., Kahn, S., Nelde, A., Stroh, T., Handel, M., et al. (2015). “The antigenic landscape of multiple myeloma: mass spectrometry (re)defines targets for T-cell-based immunotherapy” Blood 126, 1203-1213.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

LENGTHY TABLES
The patent application contains a lengthy table section. A copy of the table is available in electronic form from the USPTO web site (http://seqdata.uspto.gov/?pageRequest=docDetail&DocID=US20190346442A1). An electronic copy of the table will also be available from the USPTO upon request and payment of the fee set forth in 37 CFR 1.19(b)(3).

1. A method of generating an HLA-allele specific binding peptide sequence database comprising:
(a) providing a population of cells expressing a single HLA allele;
(b) isolating HLA-peptide complexes from said cells;
(c) isolating peptides from said HLA-peptide complexes; and
(d) sequencing said peptides.

2. The method of claim 1, which is a method of generating an HLA class I-allele specific binding peptide sequence database comprising:
(a) providing a population of cells expressing a single HLA class I allele;
(b) isolating class I HLA-peptide complexes from said cells;
(c) isolating peptides from said HLA-peptide complexes; and
(d) sequencing said peptides.

3. The method of claim 1, which is a method of generating an HLA class II-allele specific binding peptide sequence database comprising:
(a) providing a population of cells expressing a pair of HLA Class II genes, consisting of one a and one _{R} subunit;
(b) isolating class II HLA-peptide complexes from said cells;
(c) isolating peptides from said HLA-peptide complexes; and
(d) sequencing said peptides.

4. The method of claim 1, wherein said sequencing is performed by LC-MS/MS.

5. The method of claim 1, wherein the population of cells comprises at least 10⁷ cells.

6. The method of claim 1, wherein the cells are dendritic cells, macrophages or B-cells.

7. The method of claim 1, wherein the cells are tumor cells.

8. The method of claim 1, wherein the cells are contacted with an agent or condition prior to isolating said HLA-peptide complexes from said cells.

9. The method of claim 8, wherein said agent or condition is an inflammatory cytokine, a chemical agent, a therapeutic agent or radiation.

10. The method of claim 1, wherein the HLA allele is a mutated HLA allele.

11. The method of claim 1, wherein the HLA allele is selected from A*01:01, A*02:01, A*02:03, A*02:04, A*02:07, A*03:01, A*24:02, A*29:02, A*31:01, A*68:02, B*35:01, B*44:02, B*44:03, B*51:01, B*54:01, B57:01, C*03:02, C*03:04, C*04:01, C*05:01, C*06:02, C*08:01, C*08:02, C*12:02, C*14:02, C*14:03, C*15:02, and C*16:01.

12. The method of claim 1, wherein step (b) comprises lysing the cells and isolating the HLA-peptide complexes by immunoprecipitation.

13. The method of claim 1, which comprises carrying out steps (a) to (d) for different HLA alleles.

14. An HLA-allele specific binding peptide sequence database obtained by carrying out the method of claim 1.

15. A combination of two or more HLA-allele specific binding peptide sequence databases obtained by carrying out the method of claim 1 repeatedly, each time using a different HLA-allele.

16. A method for generating a prediction algorithm for identifying HLA-allele specific binding peptides, which method comprises:
training a machine with the peptide sequence database of claim 14 or the combination of claim 15.

17. The method of claim 15, wherein the machine combines one or more linear models, support vector machines, decision trees and neural networks.

18. The method according to claim 14, wherein the variables used to train the machine comprise one or more variables selected from the group consisting of peptide sequence, amino acid physical properties, peptide physical properties, expression level of the source protein of a peptide within a cell, protein stability, protein translation rate, protein degradation rate, translational efficiencies from ribosomal profiling, protein cleavability, protein localization, motifs of host protein that facilitate TAP transport, whether host protein is subject to autophagy, motifs that favor ribosomal stalling (polyproline stretches), protein features that favor NMD (long 3′ UTR, stop codon >50 nt upstream of last exon:exon junction and peptide cleavability.

19. A method for identifying HLA-allele specific binding peptides, which method comprises analyzing the sequence of a peptide with a machine which has been trained with a peptide sequence database obtained by carrying out the method of claim 1 for said HLA-allele.

20. The method of claim 16, which method comprises:
determining the expression level of the source protein of the peptide within a cell, or the amount of RNA encoding said source protein; and
wherein the source protein expression or the amount of RNA encoding said source protein is one of the predictive variables used by the machine.

21. The method according to claim 17, wherein the expression level is determined by measuring the amount of source protein or the amount of RNA encoding said source protein.

22. A method of identifying from a given set of neo-antigen comprising peptides the most suitable peptides for preparing an immunogenic composition for a subject, said method comprising selecting from a given set of peptides a plurality of peptides capable of binding an HLA protein of the subject, wherein said ability to bind an HLA protein is determined by analyzing the sequence of peptides with a machine which has been trained with peptide sequence databases corresponding to the specific HLA-binding peptides for each of the HLA-alleles of said subject.

23. A method of identifying from a given set of neo-antigen comprising peptides the most suitable peptides for preparing an immunogenic composition for a subject, said method comprising selecting from a given set of peptides a plurality of peptides determined as capable of binding an HLA protein of the subject, ability to bind an HLA protein is determined by analyzing the sequence of peptides with a machine which has been trained with a peptide sequence database obtained by carrying out the method of claim 1.

24. A method of identifying a plurality of subject-specific peptides for preparing a subject-specific immunogenic composition, wherein the subject has a tumor and the subject-specific peptides are specific to the subject and the subject's tumor, said method comprising:
(a) whole genome or whole exome nucleic acid sequencing of a sample of the subject's tumor and a non-tumor sample of the subject;
(b) determining based on the whole genome or whole exome nucleic acid sequencing:
(i) non-silent mutations present in the genome of cancer cells of the subject but not in normal tissue from the subject, and
(ii) the HLA genotype of the subject,
wherein the non-silent mutations comprise a point, splice-site, frameshift, read-through, neoORF or gene-fusion mutation; and
(c) selecting from the identified non-silent mutations the plurality of subject-specific peptides, each having a different tumor neo-epitope that is an epitope specific to the tumor of the subject and each having a predictive score indicative of binding an HLA protein of the subject,
wherein said predictive score is determined by analyzing the sequence of peptides derived from the non-silent mutations by carrying out the method of claim 16.

25. A method of identifying a plurality of subject-specific peptides for preparing a subject-specific immunogenic composition, said method comprising selecting a plurality of subject-specific peptides, each having a different tumor neo-epitope that is an epitope specific to the tumor of the subject and each having a predictive score indicative of binding an HLA protein of the subject, wherein said predictive score is determined by analyzing the sequence of peptides derived from the non-silent mutations by carrying out the method of claim 16.

26. An immunogenic composition for use in a method of inducing a tumor specific immune response, said immunogenic composition comprising two or more peptides identified with the method according to claim 20 and a pharmaceutically acceptable carrier.

27. The immunogenic composition for use in a method of inducing a tumor specific immune response, comprising autologous dendritic cells or antigen presenting cells that have been pulsed with the two or more peptides identified with the method according to claim 20.

28. The immunogenic composition for use in a method of inducing a tumor specific immune response, comprising at least one vector capable of expressing the two or more peptides identified with the method according to claim 20.

29. The immunogenic composition according to claim 24, wherein the vector is a viral vector.

30. The immunogenic composition for use in a method of inducing a tumor specific immune response, comprising at least one vector capable of expressing the two or more peptides listed for an HLA allele listed in Tables 1A, 1B and/or 1C.

31. A peptide sequence database consisting of a set of peptides listed for an HLA allele listed in Tables 1A, 1B and/or 1C.
